(19) United States
(12) Patent Application Publication
(10) Pub. No.: US 2014/0087401 A1
(43) Pub. Date: Mar. 27, 2014

(54) Biomarkers for Assessment of the Molecular Quality in Biospecimens

(71) Applicants: Alexander Oliver Vortmeyer, New Haven, CT (US); Jie Li, Orange, CT (US)

(72) Inventors: Alexander Oliver Vortmeyer, New Haven, CT (US); Jie Li, Orange, CT (US)

(21) Appl. No.: 14/124,339

(22) PCT Filed: Jun. 7, 2012
(86) PCT No.: PCT/US12/41318

§ 371 Date: Dec. 6, 2013

Related U.S. Application Data
(60) Provisional application No. 61/494,135, filed on Jun. 7, 2011.

(51) Int. Cl. G01N 33/543

(52) U.S. Cl. CPC G01N 33/54306
USPC 435/7.92

(57) ABSTRACT

The present invention provides compositions and methods for assessing the amount of degradation in a biospecimen. The invention relates to the discovery that dynamic changes in protein biomarkers correlate to the amount of degradation in a biospecimen. The present invention also provides kits for assessing the amount of degradation in a biospecimen.

### CROSS-REFERENCE TO RELATED APPLICATIONS

This application claims the benefit of priority from U.S. Provisional Application Ser. No. 61/494,135, filed on Jun. 7, 2011, the entire disclosure of which is incorporated by reference herein as if set forth herein in its entirety.

### BACKGROUND OF THE INVENTION

Mammalian tissues are maintained in a homeostatic environment by the circulation of blood. When circulation ends—as by excision of tissue from a patient—homeostasis is disrupted and molecular degradation ensues. Lack of oxygen supply causes respiratory distress and electrolyte imbalance followed by disintegration of protein, RNA, DNA, and other biomolecules. Recent technological advances have elevated surgical cancer specimens to crucially important resources to obtain patients' individual bioinformation which is decisive for personalized treatment (Mann et al., 2005, J. Clin. Oncol. 23:5148-5154; Haas-Kogan et al., 2005, J. Natl. Cancer Inst. 97:880-887; Chang et al., 2007, Lung Cancer 58:414-417; Loi et al., 2012, Lancet Oncol. 12:1162-1168; Penault-Llorca et al., 2009, Am. J. Clin. Pathol. 132:539-548; Purdie et al., 2012, Histopathology 56:702-707; Modi et al., 2005, Cancer Invest. 23:483-487; Baker et al., Clin. Cancer Res. 11:4338-4340; Clarke et al., 1999, Clin. Pharmacokinet. 36:99-114; Stevens et al., 1987, Cancer Res. 47:5846-5852; Cho et al., 2003, Nature 421:756-760). Variable degrees of degradation, however, frequently occur in specimens before the degradation process can be effectively halted (usually by fixation or freezing techniques). Moreover, a large number of known and unknown pre-analytical variables (PAVs) determine the progress of the degradation. Since these PAVs differ from case to case, the exact state of molecular degradation within specimens is unknown (Anagnostou et al., 2010, Cancer Epidemiol. Biomarkers Prey. 19:982-991; Bai et al., 2011, Lab. Invest. 91:1253-1261; Siddiqui et al., 2010, Breast Cancer Res. 12:113; Tolles et al., 2011, Breast Cancer Res. 13:R51). Because of the complexity of PAV impact, no tools or standards exists for degradation measurement. Even following only short periods of degradation, detrimental effects on structural and biomolecular quality of human biospecimens have been widely acknowledged (Pinhel et al., 2010, Breast Cancer Res. 12:R76; De Cecco et al., 2009, BMC Cancer 12:R76; Yamashita et al., 2005, Breast Cancer Res. 7:R753-764; Cappuzzo et al., 2004, J. Nat. Cancer Inst. 96:1133-1141; Cross et al., 1990, J. Clin. Pathol. 43:597-599; Sauter et al., 2009, J. Clin. Oncol. 27:1323-1333; Hammond et al., 2012, J. Clin. Oncol. 29:e458; Albanell et al., 2009, Clin. Transl. Oncol. 11:363-375; Bartlett et al., 2012, J. Clin. Pathol. 64:649-653; Chivukula et al., 2008, Mod. Pathol. 21:363-368; Moatamed et al., 2012, Am. J. Clin. Pathol. 136:754-761; Selvarajan et al., 2003, Histochem. Cell Biol. 120:251-255; Striebel et al., 2008, Am. J. Clin. Pathol. 383-390; Werner et al., 2000, Am. J. Surg. Pathol. 24:1016-1019) and analysis of human biospecimens may therefore be affected and impact patient care.

The degradation stage of mammalian tissues after extended time of exposure to PAVs has been a central topic of forensic research (Carter et al., 2010, Forensic Sci. Int. 200:60-66; Hicks et al., 2011, J. Natl. Cancer Inst. Monogr.: 43-45; Schotsmans et al., 2011, Forensic Sci. Int. 206:e43-48; Zhou et al., J. Forensic Leg Med. 18:6-9; Shishkin et al., 1977, Vopr Med. Khim.:346-351; Qizilbash, 1982, Pathol. Annu. 17 (Part 1):1-46; Mann et al., 1990, J. Forensic Sci. 35:103-111; Shean et al., 1993, J. Forensic Sci. 38:938-949; Komar et al., 1998, Alberta J. Forensic Sci. 43:57-61; Slavin et al., 2001, Qual. Manag. Health Care 10:45-53). Forensic research developed various methodologies to assess decay stage by measurement of chemicals, fatty acid, and PCR length (Vass et al., 2002, J. Forensic Sci. 47:542-553; Zur Nedden et al., 2009, Anal. Biochem. 388:108-114; Szathmary et al., 1985, Z Rechtsmed. 94:273-287). These efforts, however, predominantly investigated degradation periods in excess of several days and have minor applicability to surgical specimens. Only more recently, evidence accumulated that biochemical degradation of tissues can be observed within hours or even minutes after tissue excision (Zur Nedden et al., 2009, Anal. Biochem. 388:108-114; Laster et al., 1968, Fed. Proc. 27:1328-1330; Wildberger et al., 1963, Proc. Soc. Exp. Biol. Med. 112:168-170; Sørensen et al., 1984, Biomed. Pharmacother. 38:458-461; Micke et al., 2006, Lab. Invest. 86:202-211; Kingsbury et al., 1995, Brain Res. Mol. Brain. Res. 28:311-318; Gunther et al., 2012, Clin. Chim. Acta. (in press); Park et al., 2006, Clin. Chem. 52:988-994; Weesendorp et al., Vet. Microbiol. 141:275-281; Srinivasan et al., 2002, Am. J. Pathol. 161:1961-1971; Espina et al., 2008, Mol. Cell. Proetomics 7:1998-2018; Jung et al., 2000, Clin. Chem. Lab. Med. 38:1271-1275; Damen et al., 1998, J. Virol. Methods 72:175-184; Dickover et al., 1998, J. Clin. Microbiol. 36:1070-1073; Duchassaing, 1997, Ann. Biol. Clin. (Paris), 55:497-508; Busch et al., 1992, Transfusion 32:420-425; Rose et al., 1991, Australas Radiol. 35:145-147; Dresse et al., 2008, Tumour Biol. 29:35-40; R^{a1} et al., 2005, Proteomics 5:3262-3277; Steiniche et al., 2008, Ugeskr Laeger 170:1050; Tammen et al., 2008, Methods M01. Biol. 428:35-42). However, no degradation measurement tool exists currently, and analysis of fresh and archival human biospecimens is essentially performed without quality control.

In the clinic, analysis of human cancer specimens is important for diagnosis and therapeutic strategy selection (Mann et al., 2005, J. Clin. Oncol. 23:5148-5154; Haas-Kogan et al., 2005, J. Natl. Cancer Inst. 97:880-887; Chang et al., 2007, Lung Cancer 58:414-417; Loi et al., 2012, Lancet Oncol. 12:1162-1168; Penault-Llorca et al., 2009, Am. J. Clin. Pathol. 132:539-548; Purdie et al., 2012, Histopathology 56:702-707). In translational research, analysis of human cancer specimens is essential for new drug discovery (Modi et al., 2005, Cancer Invest. 23:483-487; Baker et al., 2005, Clin. Cancer Res. 11:4338-4340; Clarke et al., 1999, Clin. Pharmacokinet. 36:99-114; Stevens et al., 1987, Cancer Res. 47:5846-5852; Cho et al., 2003, Nature 421:756-760). Recent advances in molecular approaches have elevated surgical cancer specimens to crucially important resources to reveal patients' individual bioinformation which is decisive for personalized treatment. Variable degrees of degradation, however, frequently occur in human cancer specimens before the degradation process can be effectively retarded (usually by fixation or freezing techniques). Moreover, a large number of known and unknown PAVs determine the progress of the degradation and the molecular data obtained from these specimens may therefore be inaccurate and mislead patient care (Pinhel et al., 2010, Breast Cancer Res. 12:R76; De Cecco et al., 2009, BMC Cancer 9:409; Yamashita et al., 2005, Breast Cancer Res. 7:R753-764; Cappuzzo et al., 2004, 96:1133-1141; Cross et al., 1990, J. Clin. Pathol. 43:597-599; Sauter et al., 2009, J. Clin. Oncol. 27:1323-1333; Hammond et al., 2012, J. Clin. Oncol. 29:e458; Albanell et al., 2009, Clin. Transl. Oncol. 11:363-375; Bartlett et al., 2012, J. Clin. Pathol. 64:649-653; Chivukula et al., 2008, Mod. Pathol. 136:754-761; Moatamed et al., 2012, Am. J. Clin. Pathol. 136:754-761; Selvarajan et al., 2003, Histochem Cell Biol. 120:251-255; Streibel et al., 2008, Am. J. Clin. Pathol. 129:383-390; Werner et al., 2000, Am. J. Surg. Pathol. 24:1016-1019).

The necessity to minimize PAV impact on tissue degradation has been widely recognized. Recent work has focused on developing protocols/guidelines for better tissue manipulation and developing anti-degradation reagents such as RNA stabilizers and protease inhibitor cocktails (Loi et al., 2012, Lancet 12:1162-1168; Hammond et al., 2012, J. Clin. Oncol. 29:e458; Albanell et al., 2009, Clin. Transl. Oncol. 11:363-375; Derecskei et al., 2006, Pathol. Oncol. Res. 12:243-246; van Kemenade et al., 2007, Ned Tijdschr Geneesk 151:1283-1286; Wolff et al., 2007, J. Clin. Oncol. 25:118-145; Chin et al., 2012, Can. Urol. Assoc. J. 4:13-25; Sartore-Bianchi et al., 2012, J. Clin. Pathol.; Park et al., 2006, Clin. Chem. 52:2303-2304; Mendelsohn et al., 1978 Biochim. Biophys. Acta 519:461-473). Despite these additives, specimens remain vulnerable to degradation. Due to lack of such standard, the degree of tissue degradation of a specimen cannot be measured, and no comparison can be performed between specimens. Consequently, bio-data obtained from specimens of unknown degradation may be biased or simply inaccurate. Only after taking a clear look at specimens' degradation will clinicians be able to decide if their diagnostic data are trustworthy or whether they need to make adjustments to their interpretation—therefore, compared to efforts towards tissue protection, efforts on degradation measurement may be even more elementary for patient care

There is a need in the art for a universal standard for specimen degradation measurement. The present invention addresses this unmet need in the art.

### SUMMARY OF THE INVENTION

1. The present invention relates to a method of assessing the amount of degradation in a biological sample. The method includes the steps of measuring the level of a biological sample in the biological sample, and comparing the level of the biological sample to a reference curve, wherein the reference curve correlates the level of the biological sample with the amount of degradation in the biological sample. In one embodiment, the biological sample is a protein. In one embodiment, the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2). In one embodiment, the protein is spectrin. In one embodiment, the measuring of the biological sample comprises an immunoassay. In one embodiment, the immunoassay is an ELISA. In one embodiment, the ELISA comprises antibodies which are directed toward spectrin, any spectrin isoforms, or a calpain-mediated cleavage product of spectrin. In one embodiment, the antibodies of the ELISA are directed toward epitopes selected from the group consisting of SEQ ID NO: 4 to SEQ ID NO: 32. In one embodiment, the measuring of the biological sample comprises FRET. In one embodiment, FRET comprises a peptide selected from the group consisting of a full-length spectrin peptide, a peptide of a fragment of spectrin. In one embodiment, the peptide of a fragment of spectrin contains the sequence encoding the site of calpain cleavage (SEQ ID NO: 32). In one embodiment, FRET comprises a fluorescent dye selected from the group consisting of fluorescein, rhodamine, 4-nitrobenzo-2-oxa-1,3-diazole (NBD), cascade blue, 4,4-difluoro-5,7-diphenyl-4-bora-3a,4a-diaza-s-indacene-3-propionic acid, 4,4-difluoro-5,p-methoxyphenyl-4-bora-3a,4a-diaza-s-indacene-3-propionic acid, 4,4-difluoro-5-styryl-4-bora-3a,4a-diaza-s-indacene-propionic acid, 6-carboxy-2′,4,4′,5′,7,7′-hexachlorofluorescein (HEX), 6-carboxy-X-rhodamine, N,N,N′,N′-tetramethyl-6-carboxyrhodamine, iodoacetyl-directed probes such as 5-((((2-iodoacetyl)amino)ethyl)amino)naphthalene-1-sulfonic acid (IAEDANS, used interchangeably with AEDANS), 5-carboxyfluorescein; 6-carboxyfluorescein; 6-(fluorescein-5-carboxamide)hexanoic acid; fluorescein isothiocyanate (FITC), tetramethylrhodamine isothiocyanate (TRITC), Texas Red (TR), eosin, a phycobiliprotein, cyanine dye, coumarin, R-phycoerythrin, allophycoerythrin (APC), a R-phycoerythrin (R-PE) conjugate, an Alexa Fluor dye, a quantum dot dye, maleimide-directed probes such as 4-dimethylaminoazobenzne-4′-maleimide (DABma1) and fluorescein-5-maleimide (Fmal), or a combination thereof. In one embodiment, the fluorescent dye is HEX. In one embodiment, FRET comprises a dark quencher The method of claim 9, wherein FRET comprises a dark quencher selected from the group consisting of 4-(dimethylamino)azobenzene (Dabcyl), QSY 35, BHQ-0, Eclipse, BHQ-1, QSY 7, QSY 9, BHQ-2, ElleQuencher, Iowa Black, QSY 21, BHQ-3, or a combination thereof. In one embodiment, the fluorescent dye is HEX. In one embodiment, the dark quencher is selected from the group consisting of Dabcyl, BHQ-1, BHQ-2, and Iowa Black. In one embodiment, FRET comprises a dark quencher/fluorescent dye pair. In one embodiment, the dark quencher/fluorescent dye pair is selected from the group consisting of HEX/Dabcyl, HEX/BHQ-1, HEX/BHQ-2, and HEX/Iowa Black. In one embodiment, FRET comprises two fluorescent dyes which emit different colors of light.

The present invention also relates to a kit for assessing the amount of degradation in a biological sample. The kit includes an assay for measuring the level of a biological sample in the biological sample and comparing the level of the biological sample to a reference curve, wherein the reference curve correlates the level of the biological sample with the amount of degradation in the biological sample. In one embodiment, the biological sample is a protein. In one embodiment, the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP 1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2). In one embodiment, the protein is spectrin. In one embodiment, the assay is an ELISA. In one embodiment, the ELISA comprises antibodies which are directed toward spectrin, any spectrin isoforms, or a calpain-mediated cleavage product of spectrin. In one embodiment, the antibodies of the ELISA are directed toward epitopes selected from the group consisting of SEQ ID NO: 4 to SEQ ID NO: 32.

The present invention also relates to a kit comprising a pre-analytical variable monitor for assessing the amount of degradation in a biological sample. The kit includes an assay for measuring the level of a biological sample in the biological sample and comparing the level of the biological sample to a reference curve, wherein the reference curve correlates the level of the biological sample with the amount of degradation in the biological sample. In one embodiment, the pre-analytical variable comprises assessing pre-analytical variables extrinsic to the biological sample. In one embodiment, the pre-analytical variable comprises assessing pre-analytical variables intrinsic to the biological sample. In one embodiment, the biological sample is a protein. In one embodiment, the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2). In one embodiment, the protein is spectrin. In one embodiment, the measuring of the biological sample comprises FRET. In one embodiment, FRET comprises a peptide selected from the group consisting of a full-length spectrin peptide, a peptide of a fragment of spectrin. In one embodiment, the peptide of a fragment of spectrin contains the sequence encoding the site of calpain cleavage (SEQ ID NO: 32). In one embodiment, FRET comprises a fluorescent dye selected from the group consisting of fluorescein, rhodamine, 4-nitrobenzo-2-oxa-1,3-diazole (NBD), cascade blue, 4,4-difluoro-5,7-diphenyl-4-bora-3a,4a-diaza-s-indacene-3-propionic acid, 4,4-difluoro-5,p-methoxyphenyl-4-bora-3a,4a-diaza-s-indacene-3-propionic acid, 4,4-difluoro-5-styryl-4-bora-3a,4a-diaza-s-indacene-propionic acid, 6-carboxy-2′,4,4′,5′,7,7′-hexachlorofluorescein (HEX), 6-carboxy-X-rhodamine, N,N,N′,N′-tetramethyl-6-carboxyrhodamine, iodoacetyl-directed probes such as 5-((((2-iodoacetyl)amino)ethyl)amino)naphthalene-1-sulfonic acid (IAEDANS, used interchangeably with AEDANS), 5-carboxyfluorescein; 6-carboxyfluorescein; 6-(fluorescein-5-carboxamide)hexanoic acid; fluorescein isothiocyanate (FITC), tetramethylrhodamine isothiocyanate (TRITC), Texas Red (TR), eosin, a phycobiliprotein, cyanine dye, coumarin, R-phycoerythrin, allophycoerythrin (APC), a R-phycoerythrin (R-PE) conjugate, an Alexa Fluor dye, a quantum dot dye, maleimide-directed probes such as 4-dimethylaminoazobenzne-4′-maleimide (DABma1) and fluorescein-5-maleimide (Fmal), or a combination thereof. In one embodiment, the fluorescent dye is HEX. In one embodiment, FRET comprises a dark quencher The method of claim 9, wherein FRET comprises a dark quencher selected from the group consisting of 4-(dimethylamino)azobenzene (Dabcyl), QSY 35, BHQ-0, Eclipse, BHQ-1, QSY 7, QSY 9, BHQ-2, ElleQuencher, Iowa Black, QSY 21, BHQ-3, or a combination thereof. In one embodiment, the dark quencher is selected from the group consisting of Dabcyl, BHQ-1, BHQ-2, and Iowa Black. In one embodiment, FRET comprises a dark quencher/fluorescent dye pair. In one embodiment, the dark quencher/fluorescent dye pair is selected from the group consisting of HEX/Dabcyl, HEX/BHQ-1, HEX/BHQ-2, and HEX/Iowa Black. In one embodiment, FRET comprises two fluorescent dyes which emit different colors of light.

The present invention also relates to a kit for assessing the amount of degradation in a biological sample. The kit includes an indicator strip for measuring the level of a biological sample in the biological sample and comparing the level of the biological sample to a reference curve, wherein the reference curve correlates the level of the biological sample with the amount of degradation in the biological sample.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1, comprising FIGS. 1A-1C, is a series of photographs depicting the impact of tissue degradation on diagnostic features of surgical specimens (numeric values indicate cold ischemia time (CIT) hours). One intracranial meningioma specimen with homogeneous histology was divided into four equal-sized fractions. Tissue fractions were randomly assigned to O-hour, 4-hour, 24-hour, and 36-hour CIT exposure, followed by O.C.T. embedding to form a frozen Tissue Micro-Array (TMA). FIG. 1A is a series of photographs depicting the decrease of histological quality at time points 0 h (left upper), 4 h (right upper), 24 h (left lower) and 36 h (right lower) including reduction of nuclear staining and loss of distinct cytoplasmic borders in tissue samples. A low power image of the respective tumor sample is demonstrated in insets showing the entire TMA section with respective tumor sample in green frame. FIG. 1B is a photograph of a gel depicting tissues collected from individual pieces of the TMA were subjected to Western blotting analysis using anti-EGFR antibody. 170 kDa EGFR expression was detected in Time 0 but undetectable at later time points; lower panel: beta-actin for loading control. FIG. 1C is a photograph of a gel depicting p53-potive 293T cell line was used to observe p53 changes during tissue degradation. Western analysis exhibits significant p53 degradation during CIT exposure; lower panel: beta-actin for loading control.

FIG. 2, comprising FIGS. 2A-2E, depicts two-dimensional difference gel electrophoresis (2D DIGE) proteomic profiling and comparison. FIG. 2A is a photograph of one of the twelve 2D DIGE images obtained from Typhoon scanner using 293T cells (CIT=0 hour). Red-framed region of all 12 experimental groups is demonstrated in high resolution with DeCyder software (FIGS. 2B-C). FIG. 2B is an image, after alignment, depicting the same protein spot pink-circled in all 12 gels (gridded windows). FIG. 2C is an image (similar to FIG. 2B) of a neighboring protein spot highlighted in 12 gels. FIG. 2D is a table illustrating the gel layout in FIGS. 2B-2C. The quantitative decrease of the protein spot in FIG. 2B and the quantitative accumulation of the protein spot in FIG. 2C are observed during CIT exposure. FIG. 2E is a graphic depicting a reciprocal change between the protein spot in FIG. 2B (highlighted peak) and the protein spot in FIG. 2C (right most peak) illustrated in a three dimensional format.

FIG. 3, comprising FIGS. 3A-3F, depicts two dimensional (2D) Western blotting demonstrating changes in the proteins' isoelectric point (PI) during tissue degradation. FIG. 3A is a series of photographs depicting two EEF2 isoforms showing reciprocal changes during tissue degradation (CIT hours are labeled on the left). A quantitative ratio between the left spot (light arrow) and the right spot (dark arrow) is associated with respective CIT impact, as indicated by the logarithmical-trend-line in FIG. 3C. FIG. 3B is a photograph depicting the gel of a 1D western blot of EEF2 over time, using identical cell lysate under identical experimental conditions as in FIG. 3A. FIG. 3C is a graph depicting CIT impact, as indicated by the logarithmical-trend-line. FIG. 3D is a graph depicting how 1D Western blot is not able to distinguish EEF2 changes during tissue degradation. FIG. 3E is a series of photographs depicting a reciprocal change of the two isoforms (spot A and B, indicated with blue and red arrow, respectively) of protein PPP2C1 observed during tissue degradation. FIG. 3F is a graph depicting the logarithmical trend line indicating the association between the quantitative ratio of PPP2C1 isoforms and the CIT impact.

FIG. 4, comprising FIGS. 4A-4D, depicts protein quantity change during degradation (numeric values indicate CIT hours) in one dimensional (1D) Western blots. FIG. 4A is a photograph depicting the quantity of B23 protein decreases during CIT exposure in three meningioma specimens (tumor 1:1&2 lane, tumor 2: 3&4 lanes, and tumor 3: 5&6 lanes). FIG. 4B is a photograph depicting how commercially available anti-AHNAK antibody cannot yield a discrete immune-signal of this super-sized protein (630 kDa) in a meningioma specimen but can react with protein products of smaller molecular size (likely AHNAK breakdown products). A relatively intact pattern of AHNAK can still be observed in early CIT exposure while this intact pattern fades in later stages of tissue degradation. FIG. 4C is a photograph depicting an accumulation of ubiquitin-activating enzyme E1 (UBA1) at its proper 110-kDa size during 293T cell degradation. FIG. 4D is a photograph depicting how beta-actin is strongly and continuously expressed at all observed time points.

FIG. 5, comprising FIGS. 5A-5B, depicts the dynamic conversion between the intact form (285 kDa) and its breakdown product (150 kDa) in a 1D Western blot of alpha-II spectrin during tissue degradation in a meningioma specimen. FIG. 5A is a photograph depicting Western blotting (CIT hours are labeled on top of each lane). FIG. 5B is a graph depicting the exponential trend line indicating the strong association between the spectrin intact/breakdown ratio and the CIT impact.

FIG. 6, comprising FIGS. 6A-6H, depicts how the dynamic conversion between the intact and breakdown forms of spectrin is confirmed in multiple tissues of varied origins via Western analysis.: FIG. 6A is a photograph depicting a Western blot using a non-tumorous human kidney specimen. FIG. 6B is a photograph depicting a Western blot of three human meningioma specimens (tumor 1: 1&2 lane, tumor 2: 3&4 lanes, and tumor 3: 5&6 lanes). FIG. 6C is a photograph depicting a Western blot of a human fibroid specimen. FIG. 6D is a photograph depicting a Western blot of a mouse uterus. FIG. 6E is a photograph depicting a Western blot of mouse intestine. FIG. 6F is a photograph depicting a Western blot of mouse lung. FIG. 6G is a photograph depicting a Western blot of mouse bladder tissues. In each Western blot, the CIT hours are labeled on top of each lane. FIG. 6H is a graph depicting an exponential trend line indicating the overall association between the spectrin intact/breakdown ratio and the CIT impact in all tissues observed. For surgical specimens in which it was difficult to collect zero CIT materials, time zero is the time point that the specimens were received. Therefore, all quantitative readouts were normalized toward the highest time zero ratio (3.84, in FIG. 6F, mouse lung) and the normalized data was used to generate the trend line.

FIG. 7, comprising FIGS. 7A-7D, depicts the exploration of using spectrin intact/breakdown ratio as a TDI for autopsy tissues and FFPE tissues. FIG. 7A is a photograph depicting a Western blot where instead of CIT, post mortem intervals (PMIs) are labeled on top of each lane. FIG. 7B is a graph depicting the association between spectrin breakdown ratio and PMI as indicated by an exponential trend line. FIG. 7C is a photograph depicting the tissue fractions of a fibroid specimen after exposure to assigned CITs followed by 12-hour formalin fixation and routine paraffin embedding. Western analysis shows that spectrin is difficult to be detected as clear immuno-signals in FFPE tissues, compared to the frozen tissue at lane one (upper panel); low molecular weight beta-actin can be detected using the same blot (lower panel). However, the use of the vague immune-signals at expected spectrin sizes (red arrows indicate the intact spectrins and blue arrows indicate spectrin breakdowns) still allows for the determination of the correlation between the spectrin breakdown ratio and the CIT impact in these FFPE tissues. FIG. 7D is a graph depicting the correlation between the spectrin breakdown ratio and the CIT impact in these FFPE tissues, as illustrated by the exponential trend line.

FIG. 8, comprising FIGS. 8A-8D, depicts a summary of applying calpain-mediated spectrin cleavage to specimen degradation assessment. FIG. 8A is a photograph of a gel depicting how a Western blot detects dynamic spectrin cleavage in a cancer specimen undergoing degradation process. FIG. 8B is a photograph depicting a gel stained with Coomassie's blue stain. Purified spectrins undergo calpain-mediated dynamic cleavage in a test tube due to ambient PAV impact. FIG. 8C is an illustration depicting (fluorescence resonance energy transfer (FRET)-based spectrin cleavage detection. Application of the present invention will yield the first standard in the field for degradation measurement. FIG. 8D is a photograph of a gel depicting how preliminary data indicates that endogenous calpains cleaves extrinsic spectrins with similar kinetics to endogenous spectrins.

FIG. 9, comprising FIGS. 9A-9E, depicts the degradation-dependent spectrin cleavage to be calpain mediated. FIG. 9A is a photograph of a Western blot depicting cleavage during meningioma degradation. FIG. 9B is a graph depicting the exponential trend line of the intact:cleavage ratio. FIG. 9C is a photograph of a western blot depicting a calpain inhibitor assay. FIG. 9D is a series of photographs depicting (left to right) Western blots of human kidney, mouse lung, and after brain autopsy (PMI). FIG. 9E is an illustration depicting a schematic map of the human alpha II spectrin and calpain cleavage site.

FIG. 10 is a series of photographs of gels depicting calpain-mediated spectrin cleavage. This preliminary data has led to the developed an “in-tube” enzymatic reaction that can exclusively imitate the dynamic process of endogenous spectrin cleavage observed in native specimens

FIG. 11, comprising FIGS. 11A-B, depicts that when purified spectrin is introduced into the intercellular space of tissue in a 22° C. time-dependent PAV model, spectrin cleavage is clearly detected within 0.5 hour from intercellular fluid. FIG. 11A is a photograph of a Western blot depicting spectrin cleavage. FIG. 11B is a photograph of intercellular fluid stained with Coomassie's blue depicting the difference of protein pattern from cellular proteins.

FIG. 12, comprising FIGS. 12A-12C, depicts the principle of FRET-based spectrin cleavage in specimen degradation assessment. FIG. 12A is an illustration depicting a FRET-spectrin with a quencher in the presence of calpain. FIG. 12B is an illustration depicting FRET-spectrin with a quencher in the absence of calpain. FIG. 12C is an illustration depicting FRET-spectrin without a quencher in the presence of calpain.

FIG. 13, comprising FIGS. 13A-13B, depicts the injection/detection method with FRET-spectrin. FIG. 13A is an illustration depicting the injection of FRET-spectrin into the tissue sample. FIG. 13B is an illustration depicting the detection of the fluorescence in the tissue sample. Two types of signal/color will be captured: (red) from cleaved FRET-spectrin and (green) independent to cleavage, from control peptides.

FIG. 14 is a series of photographs depicting the use of autopsy tissue to validate spectrin as a decomposition indicator.

FIG. 15, comprising FIGS. 15A-15B, depicts examples of using surgical tissues to validate spectrin as a decomposition indicator. FIG. 15A is a series of gels depicting the conversion of spectrin to its cleavage product over time. FIG. 15B is a graph depicting the linear degradation rate of spectrin.

FIG. 16, comprising FIGS. 16A-16B, depicts examples of using mouse tissues to validate spectrin as a decomposition indicator. FIG. 16A is a series of gels depicting the conversion of spectrin to its cleavage product over time. FIG. 16B is a graph depicting the linear degradation rate of spectrin.

FIG. 17 is a schematic diagram depicting an exemplary “tissue quality assessment strip.”

FIG. 18 is a graph depicting how 2D-DIGE reveals proteomic changes during tissue decay. Proteosome alterations (over 1.5 fold difference in quantity of individual proteins) can be observed during the time course of decomposition. Among 2D-DIGE identifiable, highly-expressed protein spots (mean number: 2974), 13% have significant changes over a 48-hour ischemic time exposure.

FIG. 19, comprising FIGS. 19A-19B, depicts a representative picture of 2D-DIGE analysis using Decyder software. FIG. 19A is a series of illustrations depicting the timecourse of decomposition for 2 protein isoforms. The arros indicate the time course direction. Circled images indicate two isoforms that convert during decomposition. FIG. 19B is a series of illustration depicting 3D images indicating 2 isoforms that convert during decomposition.

FIG. 20 is a graph depicting a representative picture of the “ratio” in 2D-DIGE analysis. The quantitative ratio between two protein spots (from FIG. 19) correlates with the time-depended decay.

FIG. 21 is a series of photographs depicting the validation of proteomic findings with antibody-based assays. 2D Western blot reveals more details of protein expression than a 1D Western blot, such as the quantitative changes of isoforms and topographic isochanges. The signals from the 2D Western blot (2D WB) are correlated to the 1D Western blot (1D WB). Both methods used identical amounts of protein loading, PAGE concentration, and Western blot procedures.

FIG. 22, comprising FIGS. 22A-22B, depicts how a 2D Western blot reveals tropomyosin alteration during decay. FIG. 22A is a series of photographs of 2D Western blots depicting tropomyosin changes over time. FIG. 22B is a graph depicting the quantity of tropomyosin over the time period of decomposition.

FIG. 23, comprising FIGS. 23A-23B, depicts how a 2D Western blot reveals isoform conversion of NACA. FIG. 23A is a series of photographs of 2D Western blots depicting isoform A (arrows on left) and isoform B (arrows on right) conversion over time. FIG. 23B is a graph depicting the quantitative ration between isoform A and isoform B.

FIG. 24, comprising FIGS. 24A-24B, depicts how the incorporation of multiple indicators sensitizes the decay curve. FIG. 24A is a graph depicting the decay curve of PPPC2 based on the quantity ration of PPC2 isoform A/B over the time period of tissue decomposition. FIG. 24B is a graph depicting the decay curve of tropomyosin based on the ratio of the quantity of tropomyosin over the time period of tissue decomposition. FIG. 24C is a graph depicting the virtual decay curve using combined PPPC2 and tropomyosin data (curve formula=[PPC2 spot B]/[PPC2 spot A]×[tropomyosin spot C].

FIG. 25 is a photograph of a 1D Western blot depicting how the use of multiple antibodies also measures decay. The rapid degradation of B23 is compared to the actin control. The ratio between the two measurements can be used to measure decay.

FIG. 26 is a photograph of a 1D western blot how a single antibody reveals multiple signals, and how to apply the quantitative ratio between these signals to measure decay. The increase of UBI quantity is compared to another stable signal detected by the same antibody. The ration between the two measurements can be used to measure decay.

FIG. 27 is a graphic depicting an exemplary 2D-DIGE proteomic profiling image.

FIG. 28 is an illustration depicting the functional relationship between the identified TDIs of the invention (STRING analysis). It was found that several key elements among the discovered TDIs participate in critical steps of proteolytic degradation, including the ubiquitin-proteasome pathway.

FIG. 29 is a table depicting the functional cluster of the identified TDIs using STRING, indicating most TDIs are structural elements for cytoskeleton and conduct massive protein-protein interactions.

FIG. 30 is a series of photographs of Western blots depicting data validating the utility of tissue decay indicators in FFPE-treated tissues. Formalin fixation time was 23 hours. The level of decomposition of two fibroids was analyzed at timepoints of 2, 6, 24, and 48 hours (labeled 1-4 for fibroid 1 and 5-8 for fibroid two, respectively). Fro=frozen positive control at time T₀.

FIG. 31, comprising FIGS. 31A-31B, depicts the 2D Western blot validation of PPPC2A (meningioma). FIG. 31A is a series of photographs of 2D Western blots depicting the conversion of PPC2A isomers A and B over time. FIG. 31B is a graph depicting the ration of B/A over time.

FIG. 32, comprising FIGS. 32A-32D, depicts validation of 1D Western blots in a variety of proteins. FIG. 32A is a photograph of a 1D Western blot gel depicting the protein AHNAK (628 kDa, meningioma). FIG. 32B is a photograph of a 1D Western blot gel depicting the protein ACTB 42 kDa, 293T). FIG. 32C is a photograph of a 1D Western blot gel depicting the protein UBA1 (117 kDa, kidney). FIG. 32D is a photograph of a 1D Western blot gel depicting the protein B23 (33 kDa, four brain tumors).

FIG. 33 is an illustration depicting SPTNA1 intact and breakdown products in tissue degradation.

FIG. 34 is an illustration depicting the possible mechanisms behind tissue degradation.

FIG. 35 is an illustration depicting a schematic diagram of an ELISA quality assessment kit.

FIG. 36 is an illustration depicting the use of both intrinsic proteases and exogenic proteases (calpain) to cleave recombinated spectrin peptide for specimen quality assessment.

FIG. 37 is an illustration depicting tissue quality assessment peptides with FRET technology to examine the effect of ambient PAVs on tissue quality.

FIG. 38 is an illustration depicting an exemplary workflow using an extrinsic PAV monitor to assess tissue quality.

FIG. 39 is an illustration depicting the overall strategy for the procurement approach via molecular profiling evaluation and identification of molecular markers for tissue quality assessment.

FIG. 40 is an illustration depicting the cold ischemia time (CIT)-dependent tissue degradation model. Cold ischemic time is equal to the time the biospecimen departs from the body to the time the biospecimen receives fixatives or is frozen.

FIG. 41 is a series of photographs comparing 1D separation of proteins to 2D separation. The 2D gel electrophoresis allows for protein profiling.

FIG. 42 is a series of illustrations depicting a comparison of DNA quality in freshly frozen tissues to FFPE tissue (24 hour F-fixation).

FIG. 43 is series of illustrations depicting a comparison of RNA quality in freshly frozen tissue to FFPE tissue (24 hour F-fixation).

FIG. 44 is an illustration depicting a comparison of protein quality in in freshly frozen tissue to FFPE tissue (24 hour F-fixation).

FIG. 45 is a series of photographs depicting an exemplary time-dependent tissue decomposition model. Hematoxylin and eosin (H&E) stains are shown for morphology comparison between fractions of a meningioma with varied CIT exposure. Human cell lines of four different tissue origins, were produced and pelleted into test tubes. Examples of tissues are brain, liver, kidney, and uterus tissues. Normal human tissues from autopsies, tumor tissues from surgical resections, and mouse tissues from major organs were voxelized and randomly distributed into test tubes. The tubes were randomly assigned into different experimental groups and specimens underwent varied decay (23° C., room temperature exposure) for timepoints of 1, 4, 8, 16, 32, or 64 hours.

FIG. 46 is a series of photographs of dot blots depicting the development of spectrin antibodies for the ELISA kit.

FIG. 47 is an illustration depicting the detection of the two spectrin isoforms in two FFPE fibroid specimens (the specimens were fixed for 24 hours) and the level of decay was measured at timepoints of 2, 6, 24, and 48 hours. Fibroid one was labeled as 1-4, respectfully and fibroid two was labeled 5-8, respectively to correspond to the timepoints. 0 hour control for fibroid one is labeled as “F”.

FIG. 48, comprising FIGS. 48A-48C, depicts tissue decomposition as a time-dependent event, and that during tissue decomposition a protein undergoes time-dependent changes. During tissue decomposition, time dependent quantitative changes may be identified in a subset of proteins. Quantitative measurement of these proteins is indicative of the quality of biospecimens. FIG. 48A is an image of a Western blot of a protein that underwent a time-dependent decline during tissue decomposition. FIG. 48B is an illustration of an example of a breast specimen which underwent time-dependent decomposition. FIG. 48C is a graph of a tissue degradation indicator, wherein the quantity of the protein can represent the quality of the tissue.

FIG. 49, comprising FIGS. 49A-49B, depicts the ratio concept of the tissue degradation indicator. If using one single protein to measure decomposition, different runs or laboratories may observe different reads. FIG. 49A is a graphic depicting a series of western blots which measure the quantity of a tissue degradation indicator in one specimen in different labs. FIG. 49B is a series of images depicting the use of the quantitative ratio between two or more proteins to obtain intrinsically-stable indicators for tissue decomposition. To increase the slope (sensitivity) of the decomposition curve, proteins with largely diverse decomposition speeds are selected. FIG. 24 depicts how the incorporation of multiple indicators sensitizes the curve.

FIG. 50 is a graphic depicting how different runs or different laboratories may still get different reads when applying the “ratio” concept when tests are run separately. Methodologies should be chosen which can simultaneously capture the quantitative reads of two tissue composition indicators in order to generate a constant ratio for revealing the extent of tissue decomposition, such as a colorimetric-based approach for detecting the quantity of multiple protines (for example, gel staining with Coomassie blue, etc.), multiple antibody-based approaches, such as Western blot, 1HC, ELISA with a variety of 2 or more antibodies, and a single anti-body based approach with one antibody used for detection of multiple isoforms of one protein.

FIG. 51 is a photograph of a gel depicting how the extrinsic enzymatic reaction of calpain-mediated spectrin cleavage can imitate the tissue-intrinsic spectrin cleavage during tissue degradation for use in the extrinsic PAV monitor.

### DETAILED DESCRIPTION

The present invention relates to the discovery of biomarkers which serve as diagnostic indicators of decomposition in biospecimens. These biomarkers undergo dynamic changes during biospecimen degradation, such as conversion from an intact form of the biomarker to a cleaved form. These dynamic changes can be detected over time and correlate to the amount of degradation the biospecimen has undergone, thus directly relating the biomarkers to the quality of the biospecimen. In one embodiment, the biomarkers are cellular proteins.

The present invention also relates to systems and methods of assessing the quality of biospecimens using diagnostic biomarkers. These methods determine the impact of pre-analytical variables (PAVs) on a biospecimen through the association with the measured dynamic changes of the biomarker. In a preferred embodiment, the methods are directed toward the detection of the cleavage of biomarker human alpha II spectrin by the proteolytic enzyme calpain. The present invention also includes methods based on fluorescence resonance energy transfer (FRET) for the detection of the cleavage of human alpha II spectrin by calpain. In another embodiment, the method is based on an enzyme-linked immunosorbent assay (ELISA) to detect intact or cleaved forms of spectrin. The present invention also provides kits useful for carrying out the methods of the present invention.

### DEFINITIONS

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed methods.

The term “abnormal” when used in the context of organisms, tissues, cells or components thereof, refers to those organisms, tissues, cells or components thereof that differ in at least one observable or detectable characteristic (e.g., age, treatment, time of day, etc.) from those organisms, tissues, cells or components thereof that display the “normal” (expected) respective characteristic. Characteristics which are normal or expected for one cell or tissue type, might be abnormal for a different cell or tissue type.

A “disease” is a state of health of an animal wherein the animal cannot maintain homeostasis, and wherein if the disease is not ameliorated then the animal's health continues to deteriorate.

In contrast, a “disorder” in an animal is a state of health in which the animal is able to maintain homeostasis, but in which the animal's state of health is less favorable than it would be in the absence of the disorder. Left untreated, a disorder does not necessarily cause a further decrease in the animal's state of health.

A disease or disorder is “alleviated” if the severity of a symptom of the disease or disorder, the frequency with which such a symptom is experienced by a patient, or both, is reduced.

An “effective amount” or “therapeutically effective amount” of a compound is that amount of compound which is sufficient to provide a beneficial effect to the subject to which the compound is administered. An “effective amount” of a delivery vehicle is that amount sufficient to effectively bind or deliver a compound.

“Instructional material,” as that term is used herein, includes a publication, a recording, a diagram, or any other medium of expression which can be used to communicate the usefulness of the composition and/or compound of the invention in a kit. The instructional material of the kit may, for example, be affixed to a container that contains the compound and/or composition of the invention or be shipped together with a container which contains the compound and/or composition. Alternatively, the instructional material may be shipped separately from the container with the intention that the recipient uses the instructional material and the compound cooperatively. Delivery of the instructional material may be, for example, by physical delivery of the publication or other medium of expression communicating the usefulness of the kit, or may alternatively be achieved by electronic transmission, for example by means of a computer, such as by electronic mail, or download from a website.

The terms “patient,” “subject,” “individual,” and the like are used interchangeably herein, and refer to any animal, or cells thereof whether in vitro or in situ, amenable to the methods described herein. In certain non-limiting embodiments, the patient, subject or individual is a human.

The term “pre-analytical variable” or “PAV” refers to factors which may affect the stability of a biospecimen prior to diagnostic testing, thereby influencing the results of the testing. Examples of pre-analytical variables include pre-acquisition variables, such as the usage of antibiotics, the use of other drugs, the type of anesthesia, the duration of anesthesia, arerial clamp time, blood clamp time, blood pressure variations, intra-operative blood loss, intra-operative blood administration, intra-operative fluid administration, type of surgical procedure, medical condition. Examples of pre-analytical variables also include post-acquisition variables such as the time the sample is at room temperature, the temperature of the room the fixative, the temperature of fixation, the time duration of fixation, freezing method, freezing speed, size of the specimen, the type of container, the biomolecule extraction method, method of storage, and the storage temperature and/or duration.

The term “tissue degradation index” or “TDI” refers to a quantitative ratio between the intact molecule and its breakdown form(s) to demonstrate the degradation stage of the molecule.

Ranges: throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, and 6. This applies regardless of the breadth of the range.

The term “antibody,” as used herein, refers to an immunoglobulin molecule which is able to specifically bind to a specific epitope on an antigen. Antibodies can be intact immunoglobulins derived from natural sources or from recombinant sources and can be immunoreactive portions of intact immunoglobulins. Antibodies are typically tetramers of immunoglobulin molecules. The antibodies in the present invention may exist in a variety of forms including, for example, polyclonal antibodies, monoclonal antibodies, intracellular antibodies (“intrabodies”), Fv, Fab and F(ab)₂, as well as single chain antibodies (scFv), camelid antibodies and humanized antibodies (Harlow et al., 1999, Using Antibodies: A Laboratory Manual, Cold Spring Harbor Laboratory Press, NY; Harlow et al., 1989, Antibodies: A Laboratory Manual, Cold Spring Harbor, N.Y.; Houston et al., 1988, Proc. Natl. Acad. Sci. USA 85:5879-5883; Bird et al., 1988, Science 242:423-426). As used herein, a “neutralizing antibody” is an immunoglobulin molecule that binds to and blocks the biological activity of the antigen.

By “nucleic acid” is meant any nucleic acid, whether composed of deoxyribonucleosides or ribonucleosides, and whether composed of phosphodiester linkages or modified linkages such as phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages. The term nucleic acid also specifically includes nucleic acids composed of bases other than the five biologically occurring bases (adenine, guanine, thymine, cytosine and uracil). The term “nucleic acid” typically refers to large polynucleotides.

The term “DNA” as used herein is defined as deoxyribonucleic acid.

The term “RNA” as used herein is defined as ribonucleic acid.

The term “recombinant DNA” as used herein is defined as DNA produced by joining pieces of DNA from different sources.

The term “expression” as used herein is defined as the transcription and/or translation of a particular nucleotide sequence driven by its promoter.

“Expression vector” refers to a vector comprising a recombinant polynucleotide comprising expression control sequences operatively linked to a nucleotide sequence to be expressed. An expression vector comprises sufficient cis-acting elements for expression; other elements for expression can be supplied by the host cell or in an in vitro expression system. Expression vectors include all those known in the art, such as cosmids, plasmids (e.g., naked or contained in liposomes) and viruses (e.g., lentiviruses, retroviruses, adenoviruses, and adeno-associated viruses) that incorporate the recombinant polynucleotide.

The term “biological sample” refers to any form of naturally or commercially-available biological material, including a cell, tissue, fluid, biospecimen fluid from which biomarkers of the present invention may be assayed. For example, a biological sample may be a meat product.

The term “biospecimen” is used herein in its broadest sense. A biospecimen may be a sample of any biological tissue or fluid from which biomarkers of the present invention may be assayed. Examples of such samples include but are not limited to blood, lymph, urine, gynecological fluids, biopsies, amniotic fluid and smears. Samples that are liquid in nature are referred to herein as “bodily fluids.” Biospecimens may be obtained from a patient by a variety of techniques including, for example, by scraping or swabbing an area or by using a needle to aspirate bodily fluids. Methods for collecting various biospecimens are well known in the art. Frequently, a sample will be a “clinical sample,” i.e., a sample derived from a patient. Such samples include, but are not limited to, bodily fluids which may or may not contain cells, e.g., blood (e.g., whole blood, serum or plasma), urine, saliva, tissue or fine needle biopsy samples, and archival samples with known diagnosis, treatment and/or outcome history. Biospecimens may also include sections of tissues such as frozen sections taken for histological purposes. The sample also encompasses any material derived by processing a biospecimen. Derived materials include, but are not limited to, cells (or their progeny) isolated from the sample, proteins or nucleic acid molecules extracted from the sample. Processing of a biospecimen sample may involve one or more of: filtration, distillation, extraction, concentration, inactivation of interfering components, addition of reagents. Processing of tissue specimens for histologic examination may also include sectioning of tissue and the use of tissue staining methods and immunohistochemistry. Processing of tissue specimens for research may also include sectioning and microdissection, or coring for tissue microarrays.

The term “biomolecule” refers to any molecule produced by a living organism, such as proteins, polysaccharides, lipids, and nucleic acids.

The terms “degradation,” “decomposition,” and “decay,” which may be used interchangeably herein, refer to the breakdown of a biospecimen characterized by, for example, loss of histolological integrity, loss of integrity of diagnostic biomolecules, altered biomolecule expression.

A “biomarker” of the invention is any biological molecule which undergoes a dynamic change in response to the degradation of a biospecimen. Accordingly, using an assay to measure the level of the expression, function, or activity of a biomarker is diagnostic and prognostic of the degradation of a biospecimen. A biomarker may be detected at either the nucleic acid or protein level. In one embodiment, human alpha II spectrin is a biomarker of the invention.

The terms “human alpha II spectrin,” “spectrin,” or “SPTAN1,” which may be used interchangeably herein, refer to a cytoskeletal protein that lines the intracellular side of the plasma membrane of many cell types, forming a scaffolding and playing an important role in maintenance of plasma membrane integrity and cytoskeletal structure. In the present invention, spectrin is a preferred diagnostic biomarker of biospecimen quality.

The term “cold ischemia time” or “CIT” refers to the time between resection from the body and the halting of decay by freezing or fixation.

The term “degradation indicators” refers to biomarkers which degrade in a CIT-dependent manner and can be used to assess the state of degradation of a biospecimen.

As used herein, the terms “peptide,” “polypeptide,” and “protein” are used interchangeably, and refer to a compound comprised of amino acid residues covalently linked by peptide bonds. A protein or peptide must contain at least two amino acids, and no limitation is placed on the maximum number of amino acids that can comprise a protein's or peptide's sequence. Polypeptides include any peptide or protein comprising two or more amino acids joined to each other by peptide bonds. As used herein, the term refers to both short chains, which also commonly are referred to in the art as peptides, oligopeptides and oligomers, for example, and to longer chains, which generally are referred to in the art as proteins, of which there are many types. “Polypeptides” include, for example, biologically active fragments, substantially homologous polypeptides, oligopeptides, homodimers, heterodimers, variants of polypeptides, modified polypeptides, derivatives, analogs, fusion proteins, among others. The polypeptides include natural peptides, recombinant peptides, synthetic peptides, or a combination thereof.

### DESCRIPTION

The present invention is based on the discovery of the role of a subset of proteins in identifying the level of degradation in a biospecimen, and their utility as biomarkers for quantifying biospecimen degradation. Of the subset of proteins discovered, human alpha II spectrin has been identified as the biomarker which has the most advantageous properties to be used as marker for tissue degradation. The present invention also provides epitopes for the development of antibodies which target spectrin, as well as spectrin fragments or spectrin cleavage products.

The discovery of this subset of proteins as tissue degradation indicators provides a powerful biomarker that can be used to rapidly identify the amount of decomposition which has taken place in a biospecimen from the time of resectioning until the time of diagnostic testing. Accordingly, the invention provides compositions and methods useful in quantifying biospecimen degradation.

### Biomarkers

A biomarker is an organic biomolecule which is differentially present in a sample taken from a subject of one phenotypic status (e.g., having a disease) as compared with another phenotypic status (e.g., not having the disease). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. Therefore, they are useful as markers for disease (diagnostics), therapeutic effectiveness of a drug (theranostics) and drug toxicity.

The biomarkers of the present invention are selected from a subset of proteins identified through methods known in the art, and protocols for isolating, identifying and validating the markers are described elsewhere herein and set forth below in the Examples. The biomarkers of the present invention were identified using cold-ischemia tissue models. The present invention contemplates additional pre-analytic variable models for the identification of biomarkers such as freeze-thaw models, a comparison between normal tissue and abnormal tissue, variations of temperature, variations of tissue size, hypoxia models, storage models, and other models known by those skilled in the art. The biomarkers contemplated by the present invention are not limited to the subset of proteins disclosed herein. Any biomolecule, such as proteins, DNA or RNA, is also considered.

Most of the proteins identified by the present invention as biomarkers are highly-expressed and known to participate in degradation-related cellular events, such as membrane repair, membrane/skeleton stabilization, RNA/protein synthesis, cell death, chromosome segregation, free radical detoxification, and cell protection. These proteins undergo quantitative changes in a CIT/PAV dependent manner and therefor serve as universal tissue degradation indicators. Additionally identified were key elements in the ubiquitin-proteasome pathway including ubiquitin-like modifier activating enzyme 1 which catalyzes the first step in ubiquitin conjugation to mark cellular proteins for degradation, and two ATPases on the 26S proteasome which cleaves peptides in an ATP/ubiquitin-dependent process. In a preferred embodiment, tissue degradation indicators are biomarkers which are highly-expressed and universally-presented.

By non-limiting example, the biomarkers of the present invention may be selected from a subgroup of peptides consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP 1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin, beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2). In a preferred embodiment, the biomarker is spectrin.

The process of comparing a measured value and a reference value can be carried out in any convenient manner appropriate to the type of measured value and reference value for the biomarker of the invention. For example, “measuring” can be performed using quantitative or qualitative measurement techniques, and the mode of comparing a measured value and a reference value can vary depending on the measurement technology employed. For example, when a qualitative calorimetric assay is used to measure biomarker levels, the levels may be compared by visually comparing the intensity of the colored reaction product, or by comparing data from densitometric or spectrometric measurements of the colored reaction product (e.g., comparing numerical data or graphical data, such as bar charts, derived from the measuring device). However, it is expected that the measured values used in the methods of the invention will most commonly be quantitative values (e.g., quantitative measurements of concentration). In other examples, measured values are qualitative. As with qualitative measurements, the comparison can be made by inspecting the numerical data, or by inspecting representations of the data (e.g., inspecting graphical representations such as bar or line graphs).

The process of comparing may be manual (such as visual inspection by the practitioner of the method) or it may be automated. For example, an assay device (such as a luminometer for measuring chemiluminescent signals) may include circuitry and software enabling it to compare a measured value with a reference value for a desired biomarker. Alternately, a separate device (e.g., a digital computer) may be used to compare the measured value(s) and the reference value(s). Automated devices for comparison may include stored reference values for the biomarker(s) being measured, or they may compare the measured value(s) with reference values that are derived from contemporaneously measured reference samples.

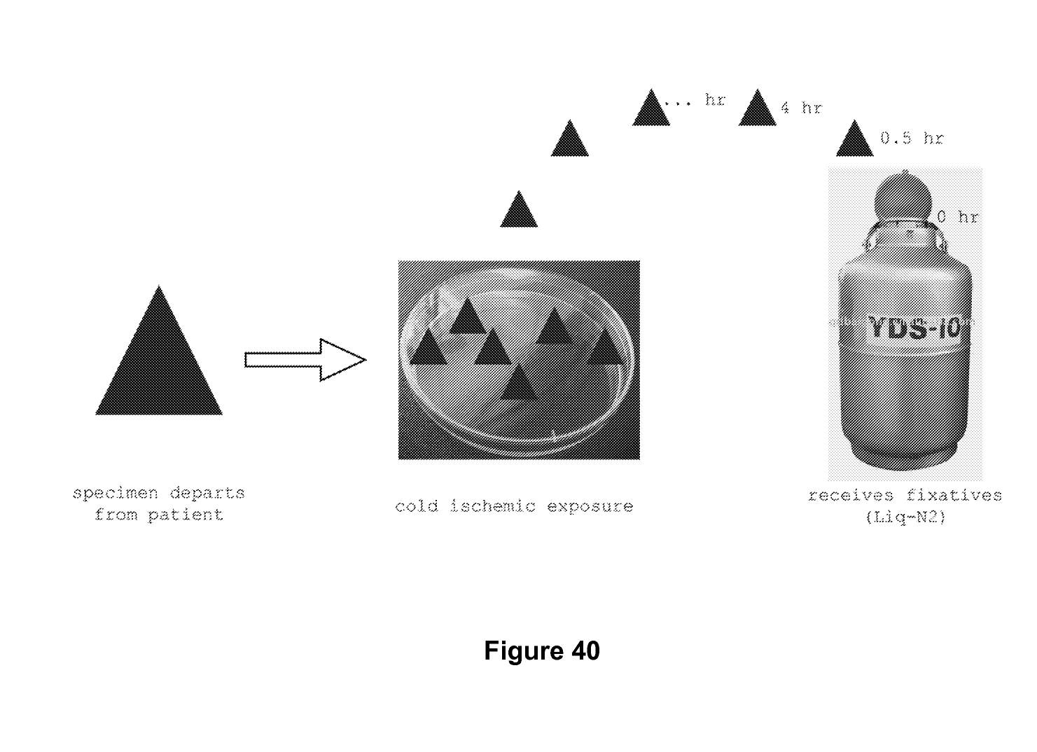

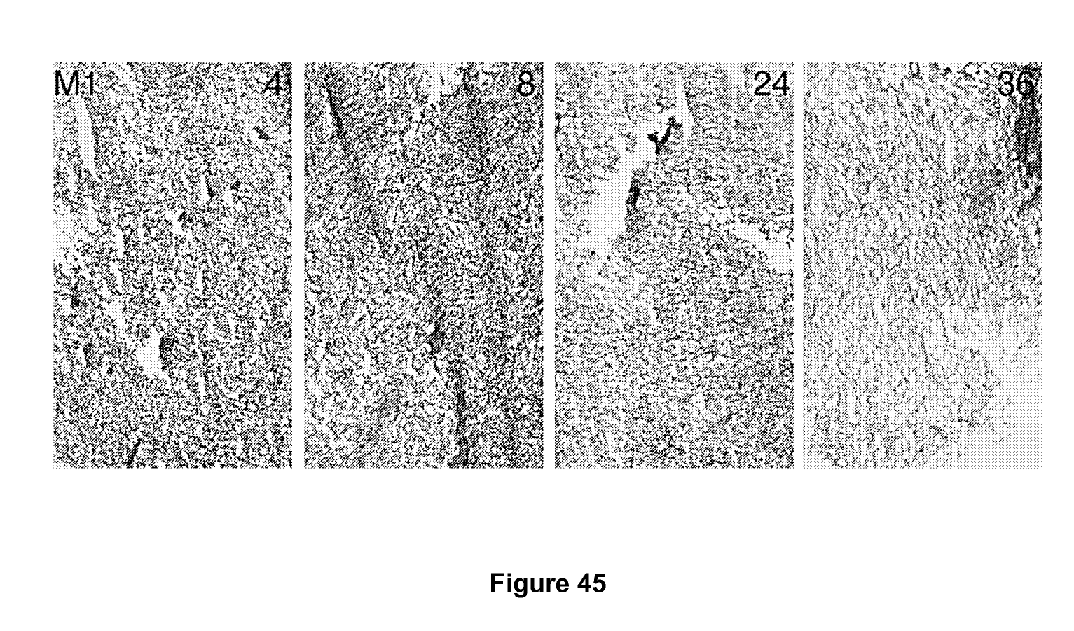

The biomarkers of the present invention undergo dynamic degradation in the CIT-dependent tissue degradation experimental model (FIG. 40). Examples of dynamic degradation include, but are not limited to, quantitative loss of the biomarker, cleavage of the biomarker into breakdown products, or isoelectric changes. The dynamic changes manifest as changes of protein quality and topographic location as the result of alterations of protein quantity, molecular mass, and isoelectric point during tissue decomposition. FIG. 45 shows an exemplary time-dependent tissue decomposition model. The degradation level of these proteins is strongly associated with the assigned CIT impact. In other embodiments, the dynamic degradation of biomarkers is correlated with additional variables, such as pH changes or changes in calcium concentrations. Therefore, these proteins may serve as tissue degradation indicators.

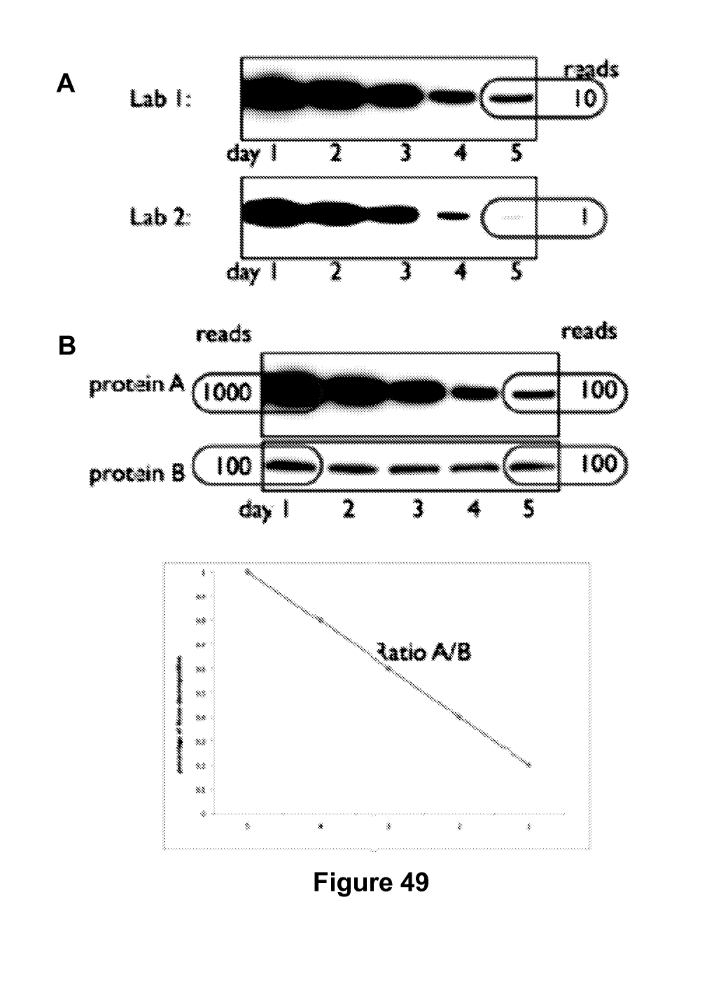

The biomarkers have been validated in surgical/autopsy specimens using the CIT-dependent tissue degradation experimental model. In other aspects of the present invention, the biomarkers are utilized as tissue degradation indicators in formalin-fixed, paraffin-embedded (FFPE) (FIG. 49). The biomarkers of the present invention may be validated through antibody based methods, as would be understood by one skilled in the art Immunossays useful for antibody-based validation include, but are not limited to, 1D Western blot, 2D western blot, 1HC, and ELISA. The present invention also provides methods for the generation of reference curves, which utilize the quantitative ratio between the intact form of a biomarker to its breakdown form(s) to demonstrate the degradation stage of the specimen.

The biomarkers of the present invention may also be used to assess the extent of decomposition in human remains to more accurately determine the time of death of the individual. In other aspects of the present invention, the biomarkers may serve as indicators for traumatic injury of tissue. In some clinical cases, such as closed craniocerebral injury, measure the presence of biomarkers in the cerebral spinal fluid (CSF) may reveal brain injury which may not be detected by MRI. In other aspects of the present invention, the biomarkers may serve as diagnostic markers for neuraldegenerative disease, such as Alzheimer's disease (AD), by identifying the biomarkers in CSF. In other aspects of the invention, the biomarkers may aid in the identification of anti-decay compositions, such as anti-calpain compounds, which may improve the preservation of both human and non-human biospecimens.

### Antibodies

The present invention provides methods for the development of antibodies directed toward spectrin (SEQ ID NO: 1) or any spectrin isoforms, such as SEQ ID NO: 2 and SEQ ID NO: 3. The spectrin antibodies may be N′-specific, C′-specific, specific to products of the calpain-mediated cleavage of spectrin, specific to the phosphorylated calpain cleavage site of spectrin, and specific to full-length spectrin. The epitopes of the present invention comprise SEQ ID NO:4 to SEQ ID NO: 32.

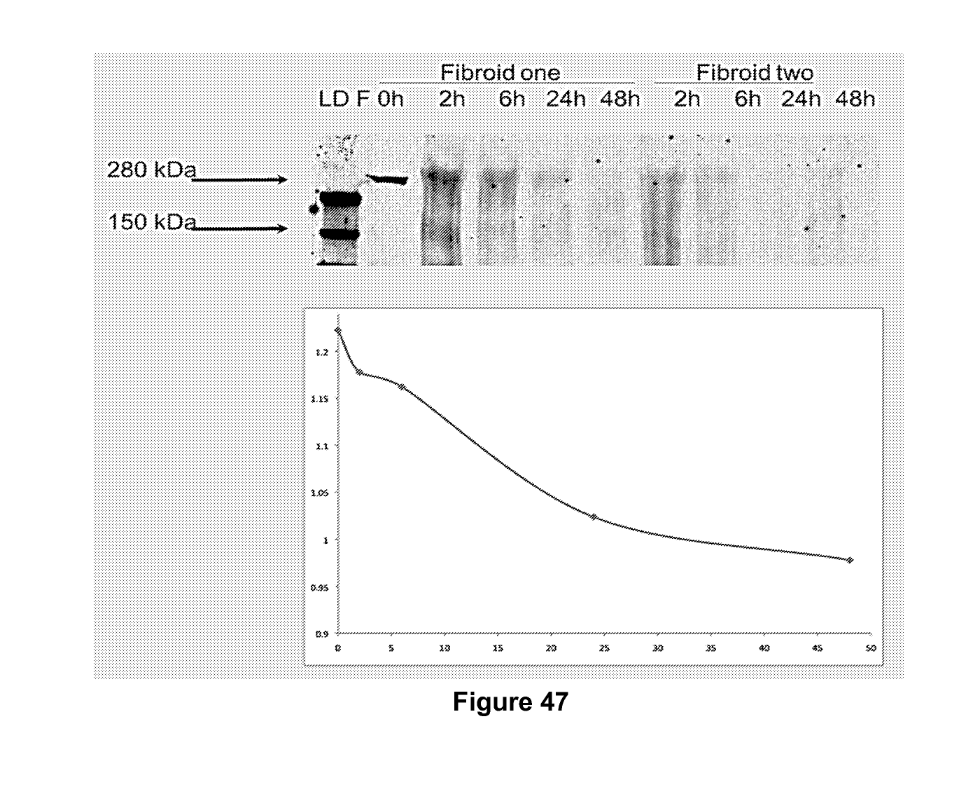

As will be understood by one skilled in the art, any antibody that can recognize and bind to an antigen of interest is useful in the present invention. The binding, function, and effect of the binding of an antibody to the antigen can then be characterized further, as described in greater detail elsewhere herein. In an embodiment, an antigen of interest is spectrin. In another embodiment, an antigen of interest is an isoform of spectrin. Isoforms may exhibit different susceptibility to decomposition. FIG. 47 shows the detection of the two spectrin isoforms in two FFPE fibroid specimens. In one aspect of the invention, an antibody is simultaneously directed toward two isoforms. In yet another embodiment, an antigen of interest is a calpain-mediated cleavage product of spectrin.

In an aspect of the invention, an antibody binds to spectrin, but not to an isoform of spectrin. In another aspect, an antibody binds to an isoform of spectrin, but not to spectrin. In yet another aspect, an antibody binds to both spectrin and an isoform of spectrin. Other antigenic proteins of the invention include other proteins identified as biomarkers for the diagnostic indication of decomposition in biospecimens. Examples of such proteins are described elsewhere herein and are set forth below in the Examples.

Methods of making and using antibodies are well known in the art. For example, polyclonal antibodies useful in the present invention are generated by immunizing rabbits according to standard immunological techniques well-known in the art (see, e.g., Harlow et al., 1988, In: Antibodies, A Laboratory Manual, Cold Spring Harbor, N.Y.). Such techniques include immunizing an animal with a chimeric protein comprising a portion of another protein such as a maltose binding protein or glutathione (GSH) tag polypeptide portion, and/or a moiety such that the antigenic protein of interest is rendered immunogenic (e.g., an antigen of interest conjugated with keyhole limpet hemocyanin, KLH) and a portion comprising the respective antigenic protein amino acid residues. The chimeric proteins are produced by cloning the appropriate nucleic acids encoding the marker protein into a plasmid vector suitable for this purpose, such as but not limited to, pMAL-2 or pCMX.

However, the invention should not be construed as being limited solely to methods and compositions including these antibodies or to these portions of the antigens. Rather, the invention should be construed to include other antibodies, as that term is defined elsewhere herein, to antigens, or portions thereof. Further, the present invention should be construed to encompass antibodies that bind to the specific antigens of interest, and they are able to bind the antigen present on Western blots, in solution in enzyme linked immunoassays, in fluorescence activated cells sorting (FACS) assays, in magenetic-actived cell sorting (MACS) assays, and in immunofluorescence microscopy of a cell transiently transfected with a nucleic acid encoding at least a portion of the antigenic protein, for example.

One skilled in the art would appreciate, based upon the disclosure provided herein, that an antibody can specifically bind with any portion of the antigen and the full-length protein can be used to generate antibodies specific therefor. However, the present invention is not limited to using the full-length protein as an immunogen. Rather, the present invention includes using an immunogenic portion of the protein to produce an antibody that specifically binds with a specific antigen. That is, the invention includes immunizing an animal using an immunogenic portion, or antigenic determinant, of the antigen.

The antibodies can be produced by immunizing an animal such as, but not limited to, a rabbit, a mouse or a camel, with an antigenic protein of the invention, or a portion thereof, by immunizing an animal using a protein comprising at least a portion of the antigen, or a fusion protein including a tag polypeptide portion comprising, for example, a maltose binding protein tag polypeptide portion, covalently linked with a portion comprising the appropriate amino acid residues. One skilled in the art would appreciate, based upon the disclosure provided herein, that smaller fragments of these proteins can also be used to produce antibodies that specifically bind the antigen of interest.

Once armed with the sequence of a specific antigen of interest and the detailed analysis localizing the various conserved and non-conserved domains of the protein, the skilled artisan would understand, based upon the disclosure provided herein, how to obtain antibodies specific for the various portions of the antigen using methods well-known in the art or to be developed.

Further, the skilled artisan, based upon the disclosure provided herein, would appreciate that using a non-conserved immunogenic portion can produce antibodies specific for the non-conserved region thereby producing antibodies that do not cross-react with other proteins which can share one or more conserved portions. Thus, one skilled in the art would appreciate, based upon the disclosure provided herein, that the non-conserved regions of an antigen of interest (e.g., spectrin) can be used to produce antibodies that are specific only for that antigen and do not cross-react non-specifically with other proteins.

The invention encompasses monoclonal, synthetic antibodies, and the like. One skilled in the art would understand, based upon the disclosure provided herein, that the crucial feature of the antibody of the invention is that the antibody bind specifically with an antigen of interest. That is, the antibody of the invention recognizes an antigen of interest (e.g., spectrin) or a fragment thereof (e.g., an immunogenic portion or antigenic determinant thereof), on Western blots, in immunostaining of cells, and immunoprecipitates the antigen using standard methods well-known in the art.

One skilled in the art would appreciate, based upon the disclosure provided herein, that the antibodies can be used to immunoprecipitate and/or immuno-affinity purify their cognate antigen as described in detail elsewhere herein, and additionally, by using methods well-known in the art.

The skilled artisan would appreciate, based upon the disclosure provided herein, that present invention includes use of a single antibody recognizing a single antigenic epitope but that the invention is not limited to use of a single antibody. Instead, the invention encompasses use of at least one antibody where the antibodies can be directed to the same or different antigenic protein epitopes.

The generation of polyclonal antibodies is accomplished by inoculating the desired animal with the antigen and isolating antibodies which specifically bind the antigen therefrom using standard antibody production methods such as those described in, for example, Harlow et al. (1988, In: Antibodies, A Laboratory Manual, Cold Spring Harbor, N.Y.).

Monoclonal antibodies directed against full length or peptide fragments of a protein or peptide may be prepared using any well-known monoclonal antibody preparation procedures, such as those described, for example, in Harlow et al. (1988, In: Antibodies, A Laboratory Manual, Cold Spring Harbor, N.Y.) and in Tuszynski et al. (1988, Blood, 72:109-115). Quantities of the desired peptide may also be synthesized using chemical synthesis technology. Alternatively, DNA encoding the desired peptide may be cloned and expressed from an appropriate promoter sequence in cells suitable for the generation of large quantities of peptide. Monoclonal antibodies directed against the peptide are generated from mice immunized with the peptide using standard procedures as referenced herein.

Nucleic acid encoding the monoclonal antibody obtained using the procedures described herein may be cloned and sequenced using technology which is available in the art, and is described, for example, in Wright et al. (1992, Critical Rev. Immunol. 12:125-168), and the references cited therein. Further, the antibody of the invention may be “humanized” using the technology described in, for example, Wright et al., and in the references cited therein, and in Gu et al. (1997, Thrombosis and Hematocyst 77:755-759), and other methods of humanizing antibodies well-known in the art or to be developed.

The present invention also includes the use of humanized antibodies specifically reactive with epitopes of an antigen of interest. The humanized antibodies of the invention have a human framework and have one or more complementarity determining regions (CDRs) from an antibody, typically a mouse antibody, specifically reactive with an antigen of interest. When the antibody used in the invention is humanized, the antibody may be generated as described in Queen, et al. (U.S. Pat. No. 6,180,370), Wright et al., (supra) and in the references cited therein, or in Gu et al. (1997, Thrombosis and Hematocyst 77(4):755-759). The method disclosed in Queen et al. is directed in part toward designing humanized immunoglobulins that are produced by expressing recombinant DNA segments encoding the heavy and light chain complementarity determining regions (CDRs) from a donor immunoglobulin capable of binding to a desired antigen, such as an epitope on an antigen of interest, attached to DNA segments encoding acceptor human framework regions. Generally speaking, the invention in the Queen patent has applicability toward the design of substantially any humanized immunoglobulin. Queen explains that the DNA segments will typically include an expression control DNA sequence operably linked to the humanized immunoglobulin coding sequences, including naturally-associated or heterologous promoter regions. The expression control sequences can be eukaryotic promoter systems in vectors capable of transforming or transfecting eukaryotic host cells or the expression control sequences can be prokaryotic promoter systems in vectors capable of transforming or transfecting prokaryotic host cells. Once the vector has been incorporated into the appropriate host, the host is maintained under conditions suitable for high level expression of the introduced nucleotide sequences and as desired the collection and purification of the humanized light chains, heavy chains, light/heavy chain dimers or intact antibodies, binding fragments or other immunoglobulin forms may follow (Beychok, Cells of Immunoglobulin Synthesis, Academic Press, New York, (1979), which is incorporated herein by reference).

One of skill in the art will further appreciate that the present invention encompasses the use of antibodies derived from camelid species. That is, the present invention includes, but is not limited to, the use of antibodies derived from species of the camelid family. As is well known in the art, camelid antibodies differ from those of most other mammals in that they lack a light chain, and thus comprise only heavy chains with complete and diverse antigen binding capabilities (Hamers-Casterman et al., 1993, Nature, 363:446-448). Such heavy-chain antibodies are useful in that they are smaller than conventional mammalian antibodies, they are more soluble than conventional antibodies, and further demonstrate an increased stability compared to some other antibodies.

Camelid species include, but are not limited to Old World camelids, such as two-humped camels (C. bactrianus) and one humped camels (C. dromedarius). The camelid family further comprises New World camelids including, but not limited to llamas, alpacas, vicuna and guanaco. The use of Old World and New World camelids for the production of antibodies is contemplated in the present invention, as are other methods for the production of camelid antibodies set forth herein.

The production of polyclonal sera from camelid species is substantively similar to the production of polyclonal sera from other animals such as sheep, donkeys, goats, horses, mice, chickens, rats, and the like. The skilled artisan, when equipped with the present disclosure and the methods detailed herein, can prepare high-titers of antibodies from a camelid species. As an example, the production of antibodies in mammals is detailed in such references as Harlow et al., (1989, Antibodies: A Laboratory Manual, Cold Spring Harbor, N.Y.). Camelid species for the production of antibodies and sundry other uses are available from various sources, including but not limited to, Camello Fataga S. L. (Gran Canaria, Canary Islands) for Old World camelids, and High Acres Llamas (Fredricksburg, Tex.) for New World camelids.

The isolation of camelid antibodies from the serum of a camelid species can be performed by many methods well known in the art, including but not limited to ammonium sulfate precipitation, antigen affinity purification, Protein A and Protein G purification, and the like. As an example, a camelid species may be immunized to a desired antigen, for example, an epitope of an antigen of the invention, or fragment thereof, using techniques well known in the art. The whole blood can them be drawn from the camelid and sera can be separated using standard techniques. The sera can then be absorbed onto a Protein G-Sepharose column (Pharmacia, Piscataway, N.J.) and washed with appropriate buffers, for example 20 mM phosphate buffer (pH 7.0). The camelid antibody can then be eluted using a variety of techniques well known in the art, for example 0.15M NaCl, 0.58% acetic acid (pH 3.5). The efficiency of the elution and purification of the camelid antibody can be determined by various methods, including SDS-PAGE, Bradford Assays, and the like. The fraction that is not absorbed can be bound to a Protein A-Sepharose column (Pharmacia, Piscataway, N.J.) and eluted using, for example, 0.15M NaCl, 0.58% acetic acid (pH 4.5). The skilled artisan will readily understand that the above methods for the isolation and purification of camelid antibodies are exemplary, and other methods for protein isolation are well known in the art and are encompassed in the present invention.

The present invention further contemplates the production of camelid antibodies expressed from nucleic acid. Such methods are well known in the art, and are detailed in, for example U.S. Pat. Nos. 5,800,988; 5,759,808; 5,840,526, and 6,015,695, which are incorporated herein by reference in their entirety. Briefly, cDNA can be synthesized from camelid spleen mRNA. Isolation of RNA can be performed using multiple methods and compositions, including TRIZOL (Gibco/BRL, La Jolla, Calif.) further, total RNA can be isolated from tissues using the guanidium isothiocyanate method detailed in, for example, Sambrook et al. (2001, Molecular Cloning, A Laboratory Manual, Cold Spring Harbor, N.Y.). Methods for purification of mRNA from total cellular or tissue RNA are well known in the art, and include, for example, oligo-T paramagnetic beads. cDNA synthesis can then be obtained from mRNA using mRNA template, an oligo dT primer and a reverse transcriptase enzyme, available commercially from a variety of sources, including Invitrogen (La Jolla, Calif.). Second strand cDNA can be obtained from mRNA using RNAse H and E. coli DNA polymerase I according to techniques well known in the art.

Identification of cDNA sequences of relevance can be performed by hybridization techniques well known by one of ordinary skill in the art, and include methods such as Southern blotting, RNA protection assays, and the like. Probes to identify variable heavy immunoglobulin chains (V_{HH}) are available commercially and are well known in the art, as detailed in, for example, Sastry et al., (1989, Proc. Nat'l. Acad. Sci. USA, 86:5728). Full-length clones can be produced from cDNA sequences using any techniques well known in the art and detailed in, for example, Sambrook et al. (2001, Molecular Cloning, A Laboratory Manual, Cold Spring Harbor, N.Y.).

The clones can be expressed in any type of expression vector known to the skilled artisan. Further, various expression systems can be used to express the V_{HH} peptides of the present invention, and include, but are not limited to eukaryotic and prokaryotic systems, including bacterial cells, mammalian cells, insect cells, yeast cells, and the like. Such methods for the expression of a protein are well known in the art and are detailed elsewhere herein.

The V_{HH} immunoglobulin proteins isolated from a camelid species or expressed from nucleic acids encoding such proteins can be used directly in the methods of the present invention, or can be further isolated and/or purified using methods disclosed elsewhere herein.

The present invention is not limited to V_{HH} proteins isolated from camelid species, but also includes V_{HH} proteins isolated from other sources such as animals with heavy chain disease (Seligmann et al., 1979, Immunological Rev. 48:145-167, incorporated herein by reference in its entirety). The present invention further comprises variable heavy chain immunoglobulins produced from mice and other mammals, as detailed in Ward et al. (1989, Nature 341:544-546, incorporated herein by reference in its entirety). Briefly, V_{H} genes were isolated from mouse splenic preparations and expressed in E. coli. The present invention encompasses the use of such heavy chain immunoglobulins in the treatment of various autoimmune disorders detailed herein.

As used herein, the term “heavy chain antibody” or “heavy chain antibodies” comprises immunoglobulin molecules derived from camelid species, either by immunization with an peptide and subsequent isolation of sera, or by the cloning and expression of nucleic acid sequences encoding such antibodies. The term “heavy chain antibody” or “heavy chain antibodies” further encompasses immunoglobulin molecules isolated from an animal with heavy chain disease, or prepared by the cloning and expression of V_{H} (variable heavy chain immunoglobulin) genes from an animal.

Once expressed, whole antibodies, dimers derived therefrom, individual light and heavy chains, or other forms of antibodies can be purified according to standard procedures known in the art.

In one embodiment of the invention, a phage antibody library may be generated, as described in detail elsewhere herein. To generate a phage antibody library, a cDNA library is first obtained from mRNA which is isolated from cells, e.g., peripheral blood lymphocytes, which express the desired protein to be expressed on the phage surface, e.g., the desired antibody. cDNA copies of the mRNA are produced using reverse transcriptase. cDNA which specifies immunoglobulin fragments are obtained by PCR and the resulting DNA is cloned into a suitable bacteriophage vector to generate a bacteriophage DNA library comprising DNA specifying immunoglobulin genes. The procedures for making a bacteriophage library comprising heterologous DNA are well known in the art and are described, for example, in Sambrook et al., supra.

Bacteriophage which encode the desired antibody, may be engineered such that the protein is displayed on the surface thereof in such a manner that it is available for binding to its corresponding binding protein, e.g., the antigen against which the antibody is directed, such as an antigen of interest. Thus, when bacteriophage which express a specific antibody are incubated in the presence of the corresponding antigen, the bacteriophage will bind to the antigen. Bacteriophage which do not express the antibody will not bind to the antigen. Such panning techniques are well known in the art and are described for example, in Wright et al. (supra).

Processes such as those described above, have been developed for the production of human antibodies using M13 bacteriophage display (Burton et al., 1994, Adv. Immunol. 57:191-280). Essentially, a cDNA library is generated from mRNA obtained from a population of antibody-producing cells. The mRNA encodes rearranged immunoglobulin genes and thus, the cDNA encodes the same. Amplified cDNA is cloned into M13 expression vectors creating a library of phage which express human Fab fragments on their surface. Phage which display the antibody of interest are selected by antigen binding and are propagated in bacteria to produce soluble human Fab immunoglobulin. Thus, in contrast to conventional monoclonal antibody synthesis, this procedure immortalizes DNA encoding human immunoglobulin rather than cells which express human immunoglobulin.

The procedures just presented describe the generation of phage which encode the Fab portion of an antibody molecule. However, the invention should not be construed to be limited solely to the generation of phage encoding Fab antibodies. Rather, phage which encode single chain antibodies (scFv/phage antibody libraries) are also included in the invention. Fab molecules comprise the entire Ig light chain, that is, they comprise both the variable and constant region of the light chain, but include only the variable region and first constant region domain (CH₁) of the heavy chain. Single chain antibody molecules comprise a single chain of protein comprising the Ig Fv fragment. An Ig Fv fragment includes only the variable regions of the heavy and light chains of the antibody, having no constant region contained therein. Phage libraries comprising scFv DNA may be generated following the procedures described in Marks et al. (1991, J. Mol. Biol. 222:581-597). Panning of phage so generated for the isolation of a desired antibody is conducted in a manner similar to that described for phage libraries comprising Fab DNA.

The invention should also be construed to include synthetic phage display libraries in which the heavy and light chain variable regions may be synthesized such that they include nearly all possible specificities (Barbas, 1995, Nature Medicine 1:837-839; de Kruif et al. 1995, J. Mol. Biol. 248:97-105).

In another embodiment of the invention, phage-cloned antibodies derived from immunized animals can be humanized by known techniques.

### METHODS OF THE INVENTION

The systems and methods of the present invention allow one to determine the quality of a biospecimen using the diagnostic biomarkers disclosed herein. The biomarkers undergo dynamic changes as the biospecimen decomposes, and these changes can be measured and quantitatively correlated with the level of cell degradation in the biospecimen. In one embodiment, the dynamic change of the biomarker is degradation, indicating a decrease in quality of the biospecimen. In another embodiment, the dynamic change of the biomarker is accumulation, indicating a decrease in quality of the biospecimen.

The levels of a biomarker of the invention may be assessed in several different biological samples. The sample may be taken from biopsy, a bodily fluid, such as blood, lymph fluid, ascites, serous fluid, pleural effusion, sputum, cerebrospinal fluid, lacrimal fluid, synovial fluid, saliva, stool, sperm and urine. The sample may also originate from a tissue, such as brain, lung, liver, spleen, kidney, pancreas, intestine, colon, mammary gland or kidney, stomach, prostate, bladder, placenta, uterus, ovary, endometrium, testicle, lymph node, skin, head or neck, esophagus, bone marrow, and blood or blood cells. Cells suspected of being transformed may be obtained by methods known for obtaining “suspicious” cells such as by biopsy, needle biopsy, fine needle aspiration, swabbing, surgical excision, and other techniques known in the art. A sample may be tissue samples or cells from a subject, for example, obtained by biopsy, intact cells, for example cells that have been separated from a tissue sample, or intact cells present in blood or other body fluid, cells or tissue samples obtained from the subject, including paraffin embedded tissue samples, proteins extracted obtained from a cell, cell membrane, nucleus or any other cellular component or mRNA obtained from the nucleus or cytosol. As used herein, the “cell from the subject” may be one or more of a renal cell carcinoma, cyst, cortical tubule, ischemic tissue, regenerative tissue, or any histological or cytological stage in-between. The cells are sometimes herein referred to as a sample. In a preferred embodiment, the sample is a tissue sample. In one embodiment, the tissue sample is isolated from a mammal In another embodiment, the mammal is human. In another embodiment, the tissue sample is isolated for the purpose of diagnosing a disease. In a further embodiment, the disease is cancer.

A biomarker can be detected by any methodology. A preferred method for detection involves first capturing the biomarker, e.g., with biospecific capture reagents, and then detecting the captured biomarkers, e.g., nucleic acids with fluorescence detection methods or proteins by mass spectrometry. Preferably, the biospecific capture reagents are bound to a solid phase, such as a bead, a plate, a membrane or a chip. Methods of coupling biomolecules, such as nucleic acids and antibodies, to a solid phase are well known in the art. They can employ, for example, bifunctional linking agents, or the solid phase can be derivatized with a reactive group, such as an epoxide or an imidizole, that will bind the molecule on contact. Biospecific capture reagents against different target proteins can be mixed in the same place, or they can be attached to solid phases in different physical or addressable locations. In one aspect, the biomarker is detected with a hand-held fluorescent detector.

Assessment of biomarker levels may encompass assessment of the level of protein concentration or the level of enzymatic activity of the biomarker, as applicable. In either case, the level is quantified such that a value, an average value, or a range of values is determined. In one embodiment, the level of protein concentration of the biomarker is quantified.

There are numerous known methods and kits for measuring the amount or concentration of a protein in a sample, including as non-limiting examples, FRET, ELISA, western blot, absorption measurement, colorimethc determination, Lowry assay, Bicinchoninic acid assay, or a Bradford assay. Commercial kits include ProteoQwest™ Colohmetric Western Blotting Kits (Sigma-Aldrich, Co.), QuantiPro™ bicinchoninic acid (BCA) Protein Assay Kit (Sigma-Aldrich, Co.), FluoroProfile™ Protein Quantification Kit (Sigma-Aldrich, Co.), the Coomassie Plus—The Better Bradford Assay (Pierce Biotechnology, Inc.), and the Modified Lowry Protein Assay Kit (Pierce Biotechnology, Inc.). In certain embodiments, the protein concentration is measured using a luminex based multiplex immunoassay panel. However, the invention should not be limited to any particular assay for assessing the level of a biomarker of the invention. That is, any currently known assay used to detect protein levels and assays to be discovered in the future can be used to detect the biomarkers of the invention. In a preferred embodiment, the method is ELISA.

Methods of quantitatively assessing the level of a protein in a biological sample such as plasma are well known in the art. In some embodiments, assessing the level of a protein involves the use of a detector molecule for the biomarker. Detector molecules can be obtained from commercial vendors or can be prepared using conventional methods available in the art. Exemplary detector molecules include, but are not limited to, an antibody that binds specifically to the biomarker, a naturally-occurring cognate receptor, or functional domain thereof, for the biomarker, or a small molecule that binds specifically to the biomarker.

In a preferred embodiment, the level of a biomarker is assessed using FRET. In one embodiment, the level of a biomarker is assessed using an antibody. Thus, non-limiting exemplary methods for assessing the level of a biomarker in a biological sample include various immunoassays, for example, immunohistochemistry assays, immunocytochemistry assays, ELISA, capture ELISA, sandwich assays, enzyme immunoassay, radioimmunoassay, fluorescent immunoassay, and the like, all of which are known to those of skill in the art. See e.g. Harlow et al., 1988, Antibodies: A Laboratory Manual, Cold Spring Harbor, N.Y.; Harlow et al., 1999, Using Antibodies: A Laboratory Manual, Cold Spring Harbor Laboratory Press, NY. The methods of the present invention comprising ELISA use the antibodies directed against the epitopes of the present invention including those of SEQ ID NO: 4 to SEQ ID NO: 32.

Other methods for assessing the level of a protein include chromatography (e.g., HPLC, gas chromatography, liquid chromatography) and mass spectrometry (e.g., MS, MS-MS). For instance, a chromatography medium comprising a cognate receptor for the biomarker or a small molecule that binds to the biomarker can be used to substantially isolate the biomarker from the biological sample. Small molecules that bind specifically to a biomarker can be identified using conventional methods in the art, for instance, screening of compounds using combinatorial library methods known in the art, including biological libraries, spatially-addressable parallel solid phase or solution phase libraries, synthetic library methods requiring deconvolution, the “one-bead one-compound” library method, and synthetic library methods using affinity chromatography selection.

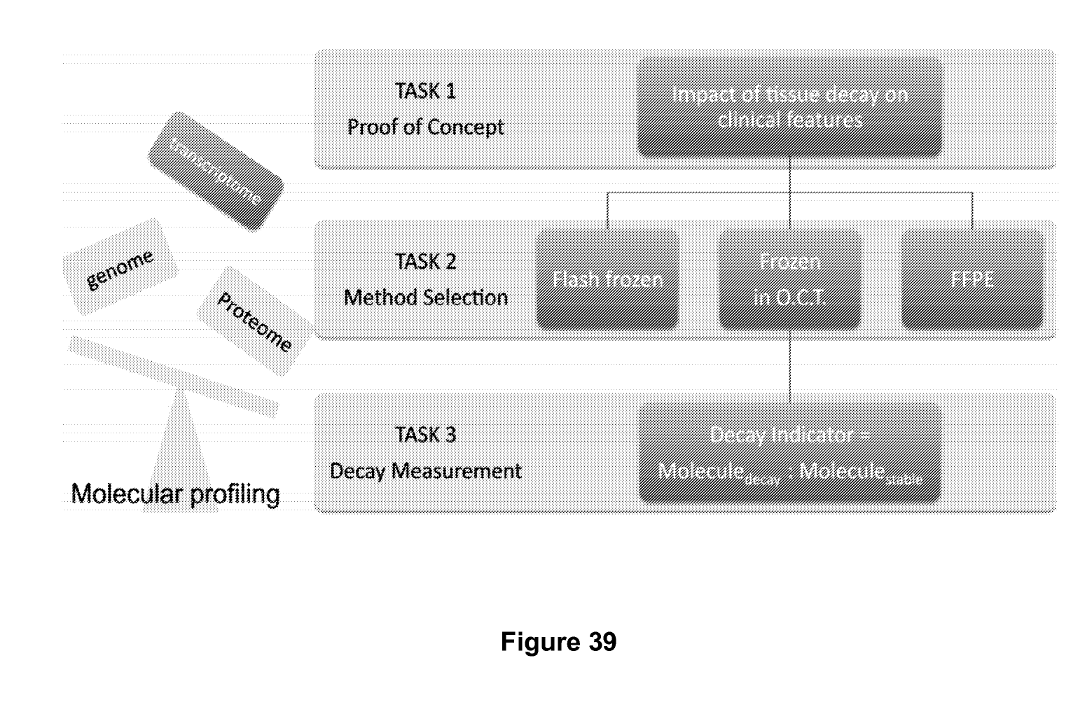

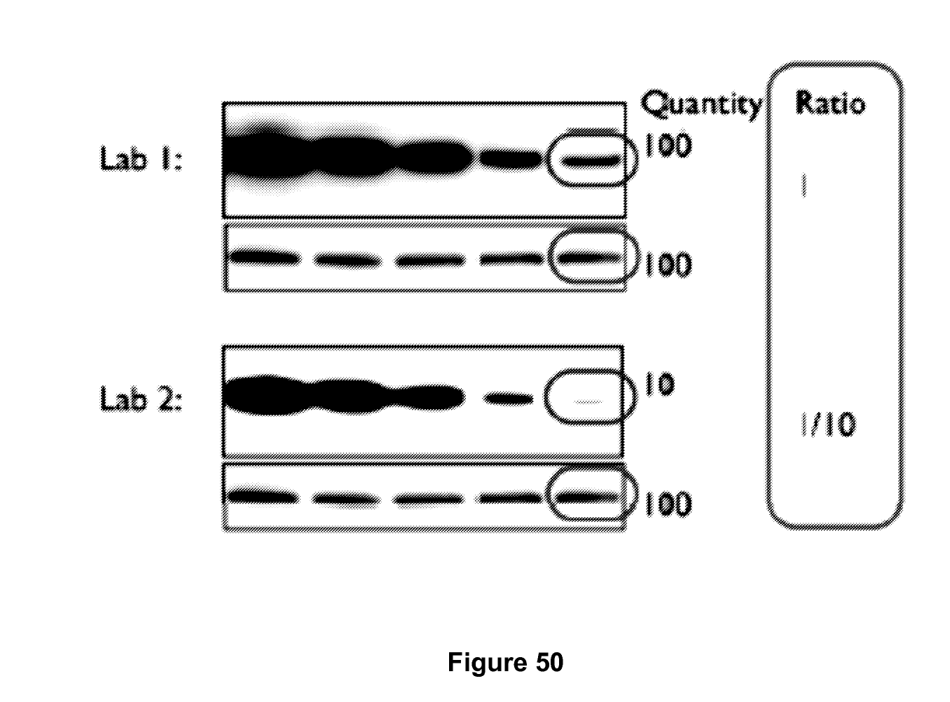

The present invention provides methods for accurate and comprehensive assessment of the degradation of a biospecimen. In one embodiment, the method comprises a comparison of the observed quantity of a biomarker to a standardized reference curve. The reference curve is calculated from the ratio of two proteins measured simultaneously against corresponding tissue decomposition. The proteins may be measured using standardized procedures, as would be understood by one skilled in the art. As shown in FIG. 50, methodologies should be chosen which can simultaneously capture the quantitative reads of two tissue composition indicators in order to generate a constant ratio for revealing the extent of tissue decomposition. In another embodiment, the method of assessment includes an overall tissue degradation index. Tissue degradation indices yield a transformative platform dedicated to specimen quality control, and addresses a critical, yet unmet need for developing a universal standard for specimen degradation measurement. This platform is well-adapted to existing specimen collection guidelines, and can be applied to assess retrospective archived specimens. The platform can be used for optimizing procurement guidelines and evaluating degradation inhibitors making it a valuable tool to standardize degradation measurement. FIG. 39 shows a procurement approach via molecular profiling evaluation and identification of molecular markers for tissue quality assessment. The overall Tissue Degradation Index is comprised of the combination of the intrinsic TDI (iTDI) and extrinsic TDI (eTDI), which are described elsewhere herein and set forth below in the Examples. TDIs can also be validated in a large cohort of specimens of different tissue types and processing modalities. A TDI degradation database can be developed through the procurement of additional data, such as the decomposition curves for individual biomarkers. The generation of a degradation curve for any biomarker of interest allows for the extrapolation of original expression intensity by correlating actual measurements with TDI readout.

The level of a substantially isolated protein can be quantitated directly or indirectly using a conventional technique in the art such as spectrometry, Bradford protein assay, Lowry protein assay, biuret protein assay, or bicinchoninic acid protein assay, as well as immunodetection methods.

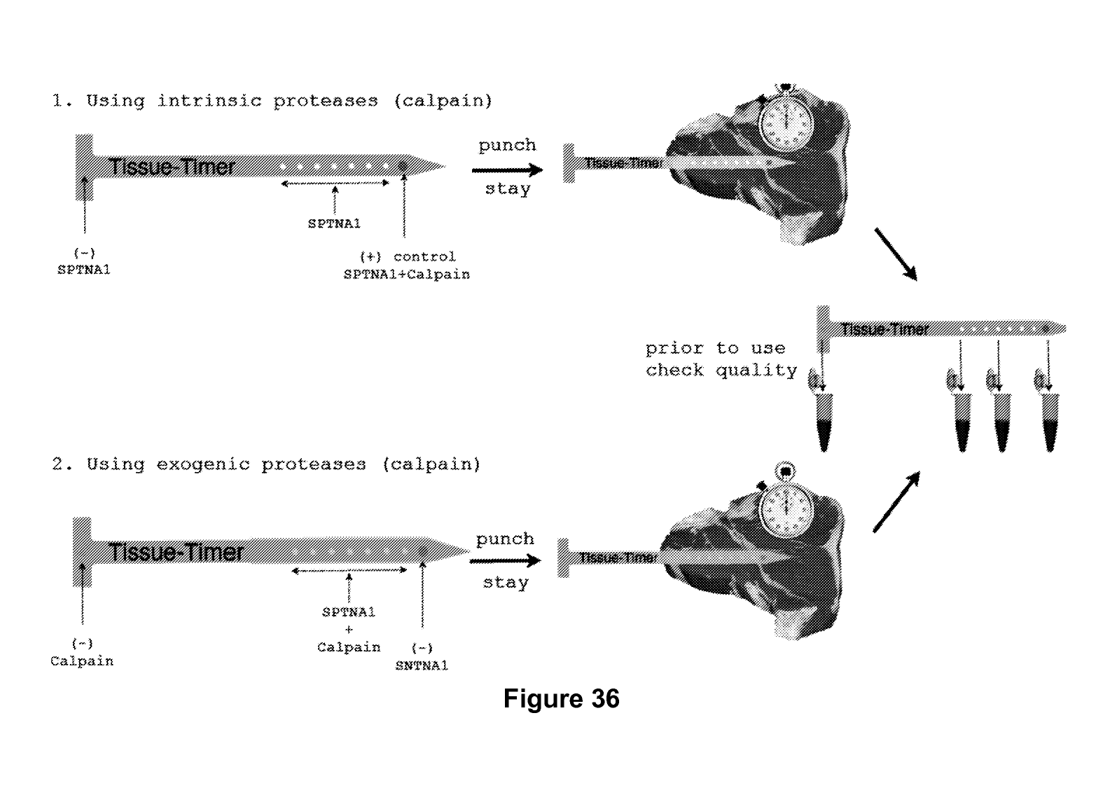

In another embodiment, the level of enzymatic activity of the biomarker if such biomarker has an enzymatic activity may be quantified. In another embodiment, the level of enzymatic cleavage of the biomarker may be quantified (FIG. 36). Generally, enzyme activity may be measured by means known in the art, such as measurement of product formation, substrate degradation, or substrate concentration, at a selected point(s) or time(s) in the enzymatic reaction. There are numerous known methods and kits for measuring enzyme activity. For example, see U.S. Pat. No. 5,654,152. Some methods may require purification of the biomarker prior to measuring the enzymatic activity of the biomarker. A pure biomarker constitutes at least about 90%, preferably, 95% and even more preferably, at least about 99% by weight of the total protein in a given sample. Biomarkers of the invention may be purified according to methods known in the art, including, but not limited to, ion-exchange chromatography, size-exclusion chromatography, affinity chromatography, differential solubility, differential centrifugation, and HPLC.

As apparent from the examples disclosed herein, diagnostic tests that use the biomarkers of the invention exhibit a sensitivity and specificity of at least 75%, at least 80%, at least 85%, at least 90%, at least 95%, at least 98% and about 100%. In some instances, screening tools of the present invention exhibits a high sensitivity of at least 75%, at least 80%, at least 85%, at least 90%, at least 95%, at least 98% and about 100%. Without wishing to be bound by any particular theory, it is believed that screening tools should exhibit high sensitivity, but specificity can be low. However, diagnostics should have high sensitivity and specificity.

### Immunoassay

Immunoassays, in their simplest and most direct sense, are binding assays. Certain preferred immunoassays are the various types of enzyme linked immunosorbent assays (ELISA), radioimmunoassays (RIA), immunoflow cytometry (IFC), and cell block immunohistochemistry (IHC) known in the art. Immunohistochemical detection using tissue sections is also particularly useful. However, it will be readily appreciated that detection is not limited to such techniques, and western blotting, dot blotting, FACS analyses, and the like may also be used.

In one exemplary ELISA, antibodies binding to the biomarker protein of the invention are immobilized onto a selected surface exhibiting protein affinity, such as a well in a polystyrene microtiter plate. Then, a test composition suspected of containing the biomarker antigen, such as a clinical sample, is added to the wells. After binding and washing to remove non-specifically bound immunecomplexes, the bound antibody may be detected. Detection is generally achieved by the addition of a second antibody specific for the target protein that is linked to a detectable label. This type of ELISA is a simple “sandwich ELISA”. Detection may also be achieved by the addition of a second antibody, followed by the addition of a third antibody that has binding affinity for the second antibody, with the third antibody being linked to a detectable label.

In another exemplary ELISA, the samples suspected of containing the biomarker antigen are immobilized onto the well surface and then contacted with the antibodies of the invention. After binding and washing to remove non-specifically bound immunecomplexes, the bound antigen is detected. Where the initial antibodies are linked to a detectable label, the immunecomplexes may be detected directly. Again, the immunecomplexes may be detected using a second antibody that has binding affinity for the first antibody, with the second antibody being linked to a detectable label.

Another ELISA in which the proteins or peptides are immobilized, involves the use of antibody competition in the detection. In this ELISA, labeled antibodies are added to the wells, allowed to bind to the biomarker protein, and detected by means of their label. The amount of marker antigen in an unknown sample is then determined by mixing the sample with the labeled antibodies before or during incubation with coated wells. The presence of marker antigen in the sample acts to reduce the amount of antibody available for binding to the well and thus reduces the ultimate signal. This is appropriate for detecting antibodies in an unknown sample, where the unlabeled antibodies bind to the antigen-coated wells and also reduces the amount of antigen available to bind the labeled antibodies.

Irrespective of the format employed, ELISAs have certain features in common, such as coating, incubating or binding, washing to remove non-specifically bound species, and detecting the bound immunecomplexes. These are described as follows:

In coating a plate with either antigen or antibody, the wells of the plate are incubated with a solution of the antigen or antibody, either overnight or for a specified period of hours. The wells of the plate are then washed to remove incompletely adsorbed material. Any remaining available surfaces of the wells are then “coated” with a nonspecific protein that is antigenically neutral with regard to the test antisera. These include bovine serum albumin (BSA), casein and solutions of milk powder. The coating of nonspecific adsorption sites on the immobilizing surface reduces the background caused by nonspecific binding of antisera to the surface.

In ELISAs, it is probably more customary to use a secondary or tertiary detection means rather than a direct procedure. Thus, after binding of a protein or antibody to the well, coating with a non-reactive material to reduce background, and washing to remove unbound material, the immobilizing surface is contacted with the control and/or clinical or biological sample to be tested under conditions effective to allow immunecomplex (antigen/antibody) formation. Detection of the immunecomplex then requires a labeled secondary binding ligand or antibody, or a secondary binding ligand or antibody in conjunction with a labeled tertiary antibody or third binding ligand.

“Under conditions effective to allow immunecomplex (antigen/antibody) formation” means that the conditions preferably include diluting the antigens and antibodies with solutions such as, but not limited to, BSA, bovine gamma globulin (BGG) and phosphate buffered saline (PBS)/Tween. These added agents also tend to assist in the reduction of nonspecific background.

The “suitable” conditions also mean that the incubation is at a temperature and for a period of time sufficient to allow effective binding. Incubation steps are typically from about 1 to 2 to 4 hours, at temperatures preferably on the order of 25° to 27° C., or may be overnight at about 4° C.

Following all incubation steps in an ELISA, the contacted surface is washed so as to remove non-complexed material. A preferred washing procedure includes washing with a solution such as PBS/Tween, or borate buffer. Following the formation of specific immunecomplexes between the test sample and the originally bound material, and subsequent washing, the occurrence of even minute amounts of immunecomplexes may be determined.

To provide a detecting means, the second or third antibody will have an associated label to allow detection. Preferably, this label is an enzyme that generates a color or other detectable signal upon incubating with an appropriate chromogenic or other substrate. Thus, for example, the first or second immunecomplex can be detected with a urease, glucose oxidase, alkaline phosphatase or hydrogen peroxidase-conjugated antibody for a period of time and under conditions that favor the development of further immunecomplex formation (e.g., incubation for 2 hours at room temperature in a PBS-containing solution such as PBS-Tween).

After incubation with the labeled antibody, and subsequent to washing to remove unbound material, the amount of label is quantified, e.g., by incubation with a chromogenic substrate such as urea and bromocresol purple or 2,2′-azido-di-(3-ethyl-benzthiazoline-6-sulfonic acid [ABTS] and H₂O₂, in the case of peroxidase as the enzyme label. Quantitation is then achieved by measuring the degree of color generation, e.g., using a visible spectra spectrophotometer.

The methods of the present invention comprising ELISA use the antibodies directed against the epitopes of the present invention including SEQ ID NO: 4 to SEQ ID NO: 32.

### Fluorescence Resonance Emission Transfer (FRET)

Fluorescence resonance energy transfer, also titled Förster resonance energy transfer and abbreviated as “FRET,” generally comprises an energy transfer that occurs between two chromophores, namely, an energy donor (a fluorophore) and an energy acceptor (optionally a fluorophore), as a result of absorption of excitation light by the energy donor. Although the present disclosure should not be limited to any theory of how FRET occurs, the energy transfer may be through a coupled dipole-dipole interaction and may be a nonradiative transfer from donor to acceptor, without generation of an intermediate photon. The efficiency of energy transfer may be strongly dependent on the separation distance between the donor and acceptor, such as varying by an inverse sixth power law. Accordingly, most FRET, for practical purposes, may be limited to a separation distance of less than about ten nanometers, such as about 30 to 100 angstroms. Also, the efficiency of energy transfer may be dependent on the spectral overlap of donor emission and acceptor absorption. In any event, the donor may be described as a fluorescent dye or a fluorophore, which may fluoresce in response to excitation by excitation light, and the acceptor also may be described as a fluorescent dye or a fluorophore, which may fluoresce in response to energy transfer from the donor, or may be described as a substantially non-fluorescent quencher of donor fluorescence.

Fluorescent dyes that are suitable for use in FRET assays include, but are not limited to, fluorescein, rhodamine, 4-nitrobenzo-2-oxa-1,3-diazole (NBD), cascade blue, 4,4-difluoro-5,7-diphenyl-4-bora-3a,4a-diaza-s-indacene-3-propionic acid, 4,4-difluoro-5,p-methoxyphenyl-4-bora-3a,4a-diaza-s-indacene-3-propionic acid, 4,4-difluoro-5-styryl-4-bora-3a,4a-diaza-s-indacene-propionic acid, 6-carboxy-2′,4,4′,5′,7,7′-hexachlorofluorescein (HEX), 6-carboxy-X-rhodamine, N,N,N′,N′-tetramethyl-6-carboxyrhodamine, iodoacetyl-directed probes such as 5-((((2-iodoacetyl)amino)ethyl)amino)naphthalene-1-sulfonic acid (IAEDANS, used interchangeably with AEDANS), 5-carboxyfluorescein; 6-carboxyfluorescein; 6-(fluorescein-5-carboxamide)hexanoic acid; fluorescein isothiocyanate (FITC), tetramethylrhodamine isothiocyanate (TRITC), Texas Red (TR), eosin, a phycobiliprotein, cyanine dye, coumarin, R-phycoerythrin, allophycoerythrin (APC), a R-phycoerythrin (R-PE) conjugate, an Alexa Fluor dye, a quantum dot dye, maleimide-directed probes such as 4-dimethylaminoazobenzne-4′-maleimide (DABma1) and fluorescein-5-maleimide (Fmal), or a combination thereof (e.g., tandem conjugates). In a preferred embodiment, the reporter dye is HEX.

In some methods of the present invention, dark quenchers are utilized in the FRET assay. Dark quenchers absorb the energy of a proximal fluorophore and emit the energy as heat rather than as light, thereby suppressing the emission of the fluorophore. Examples of dark quenchers include, but are not limited to, 4-(dimethylamino)azobenzene (Dabcyl), QSY 35, BHQ-0, Eclipse, BHQ-1, QSY 7, QSY 9, BHQ-2, ElleQuencher, Iowa Black, QSY 21, BHQ-3, or a combination thereof. In a preferred embodiment, the dark quencher is selected from the group consisting of Dabcyl, BHQ-1, BHQ-2, and Iowa Black.

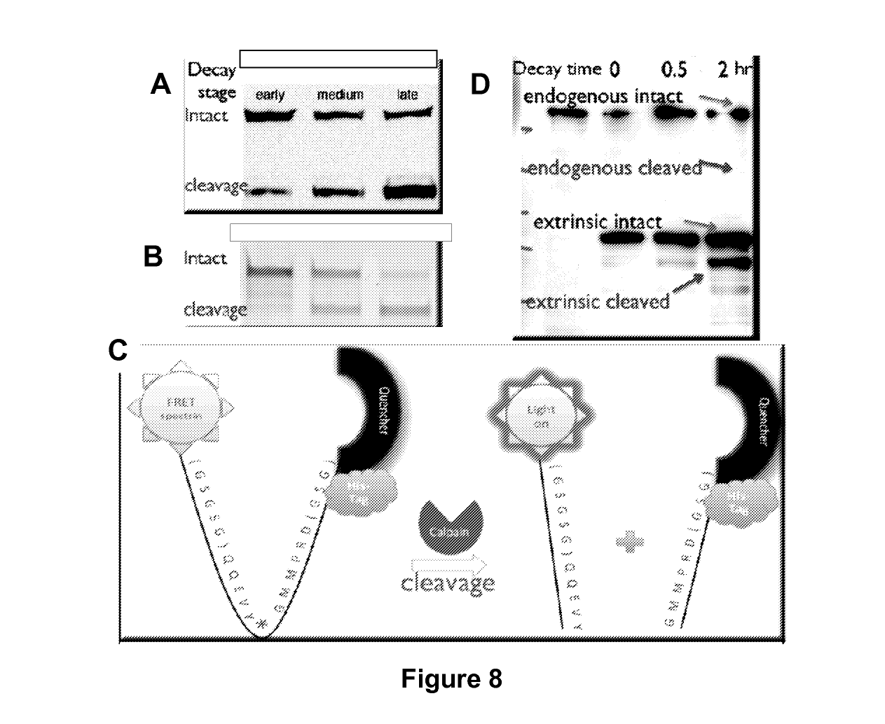

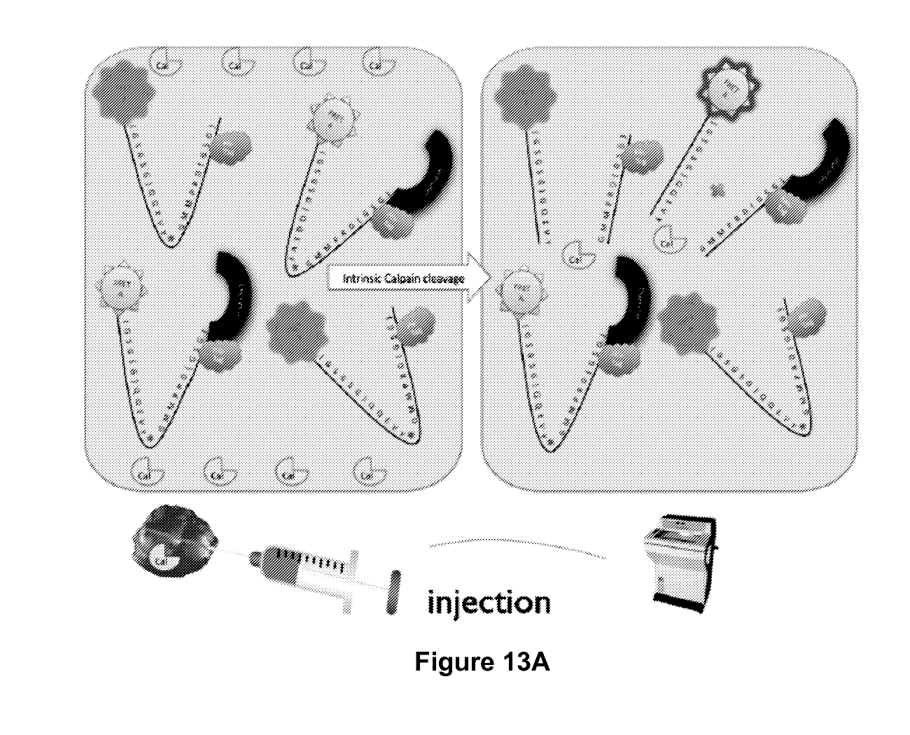

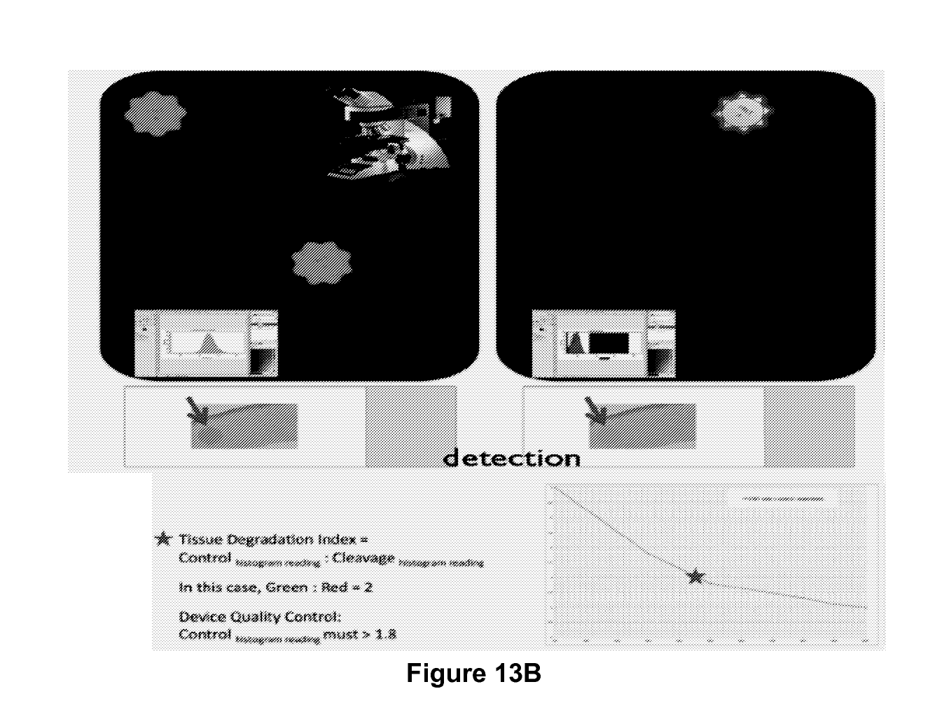

The present invention includes methods of using FRET to detect the cleavage of the biomarker spectrin by the enzyme calpain. In one embodiment, the spectrin peptide used in the FRET assay comprises the full-length spectrin peptide. In another embodiment, the spectrin peptide used in the FRET assay comprises a fragment of spectrin. In another embodiment, the fragment of spectrin contains the sequence encoding the site of calpain cleavage (SEQ ID NO: 32). In another embodiment, the sequence encoding the site of calpain cleavage is His-tagged. In determining the locations of the chromophore pairs on the spectrin, it is preferred there is a chromophore on each side of the calpain cleavage site, such that when the spectrin peptide is cleaved by calpain, each resulting fragment contains a chromophore. In one embodiment, the chromophore pair consists of a dark quencher and a fluorescent dye (FIG. 8C). Examples of quencher/fluorescent dye pairs include, but are not limited to, Oregon Green 488-X/Dabcyl, 6-FAM/Dabcyl, TET/Dabcyl, JOE/Dabcyl, HEX/Dabcyl, Cy3/Dabcyl, TAMRA/Dabcyl, ROX/Dabcyl, Texas Red/Dabcyl, Oregon Green 488-X/BHQ-1,6-FAM/BHQ-1, Rhodamine Green-X/BHQ-1, Oregon Green 514/BHQ-1, TET/BHQ-1-JOE/BHQ-1, HEX/BHQ-1, Cy3/BHQ-1, Rhodamine Red-X/BHQ-1, TAMRA/BHQ-1, HEX/BHQ-2, Cy3/BHQ-2, Rhodamine Red-X/BHQ-2, TAMRA/BHQ-2, ROX/BHQ-2, Texas Red-X/BHQ-2, Bodipy 630/650-X/BHQ-2, Bodipy 650/665-X/BHQ-2, Cy5/BHQ-2,6-FAM/Iowa Black, Rhodamine Green-X/Iowa Black, Oregon Green 514/Iowa Black, TET/Iowa Black, JOE/Iowa Black, HEX/Iowa Black, Cy3/Iowa Black, Rhodamine Red-X/Iowa Black, ROX/Iowa Black, Texas Red-X/Iowa Black, TAMRA/Iowa Black, Bodipy 630/650-X/Iowa Black, Bodipy 650/665-X/Iowa Black, and Cy5/Iowa Black. Preferred dark quencher/fluorescent dye pairs of the present invention are selected from the group consisting of HEX/Dabcyl, HEX/BHQ-1, HEX/BHQ-2, and HEX/Iowa Black. In another embodiment, the chromophore pair consists of two fluorescent dyes which emit different colors of light. (FIG. 13). For example, one fluorescent dye emits green light, while the second fluorescent dye emits red light.

### Kits

The commercial form of an analytical method is often a kit. A kit includes more than one component for the performance of the assay. It may include a substance and detailed instruction for use. In general, the kit includes all or most of the components necessary for the assay.

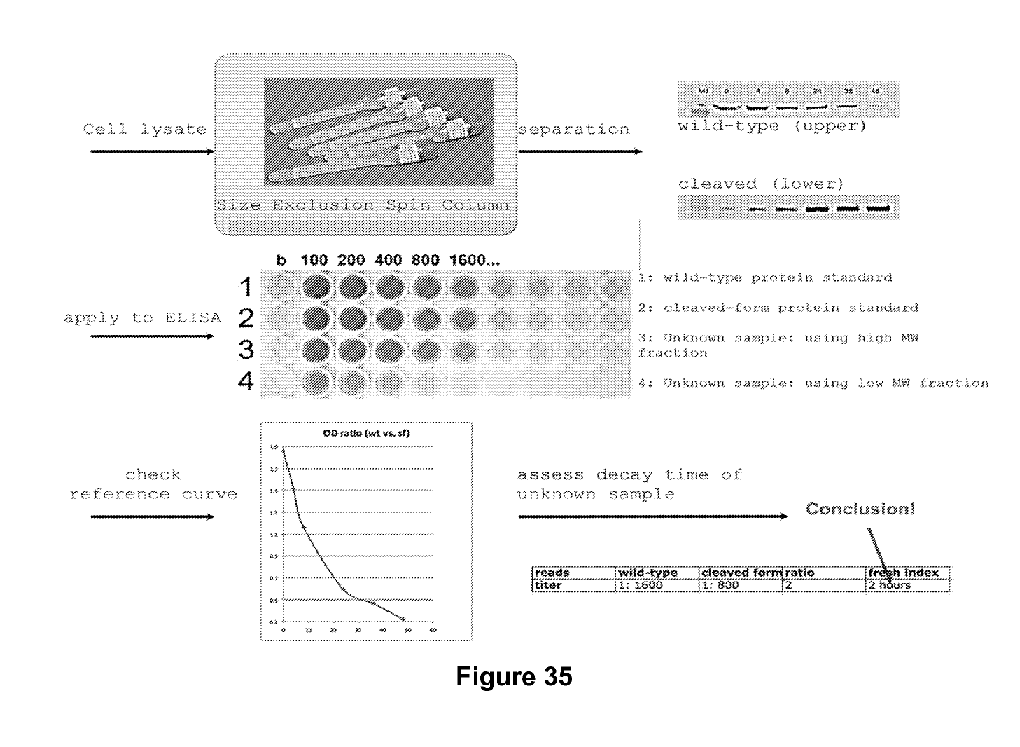

There are numerous types of methods that can be used for the analysis of a specific substance. To find the location of a substance in tissue, e.g. fluorescent or gold labeled reactive components like antibodies can be used. Different methods for detecting and quantifying a substance in various fluids include nephelometric and turbidometric methods but most particularly ELISA (FIG. 35). All these methods have detailed names after the special setting in performance and applications. The ELISA could e.g. be either presented as a sandwich method including a catcher situation and a later stage of detection of bound substance or an inhibition method where the substance to be analyzed may in a mixture react with reactive components like antibodies and the non-reactive antibodies in this mixture are later detected after reacting with e.g. immobilized pure substance. The methods of the present invention comprising ELISA use the antibodies directed against the epitopes of the present invention including SEQ ID NO: 4 to SEQ ID NO: 32, as well as antibodies currently known in the art.

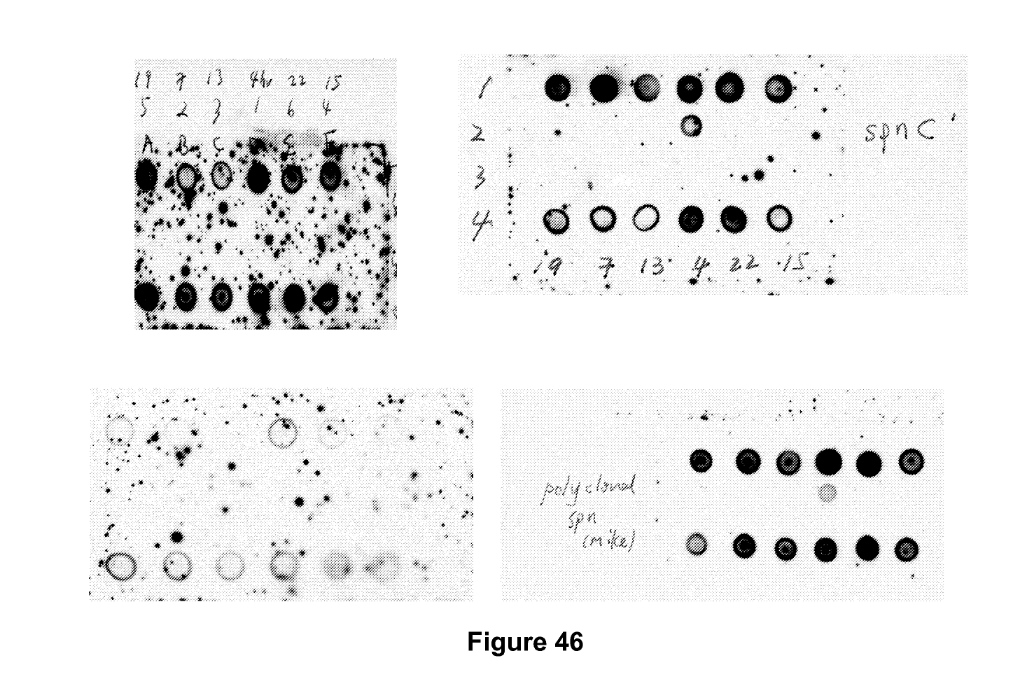

An exemplary ELISA kit of the present invention is built based on conventional “sandwich” ELISA and anti-wild type biomarker and anti-cleaved biomarker antibodies pre-bound to assigned wells of a 96-well plate. The fractions of the test lysates are serially diluted and then added to assigned rows and columns. After blotting, washing, and detection anti-wild type biomarker and anti-cleaved biomarker antibodies are added. The substrate for the conjugated enzyme for the second round of antibodies is added to yield colorimetric signals, which are read in reference to the standard rows. The status of decomposition in tested tissues is determined by comparing the experimental data with the reference curve for the specific biomarker. The kits would comprise antibodies, such as those of the present invention and those known in the art, size exclusion spin columns, a protein standard, and other general materials required for ELISA, as would be understood by one skilled in the art. FIG. 46 shows a series of photographs of dot blots depicting the development of spectrin antibodies for the ELISA kit.

The invention also relates to a kit for determining a biospecimen degradation process by a method according to the invention. In a preferred embodiment, FRET is used to detect the calpain-mediated cleavage of spectrin to indicate the degree to which a biospecimen has degraded. Spectrin was selected as the degradation indicator of choice because its dynamic conversion between intact and cleaved form allows for both internal and external assessment of the quality of the biospecimen. Spectrin is universally expressed because it is a cytoskeletal protein, and the mechanism of its calpain-mediated cleavage is well-documented in the literature. Spectrin has also been validated as a tissue decomposition indicator using various tissue specimens (FIG. 46).

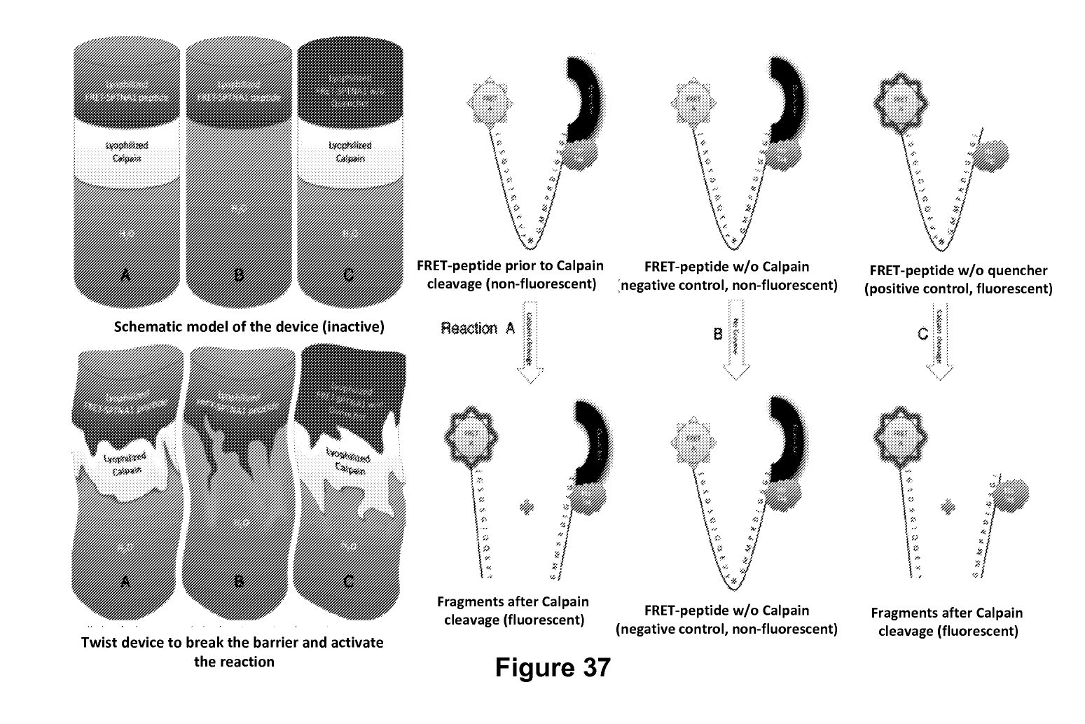

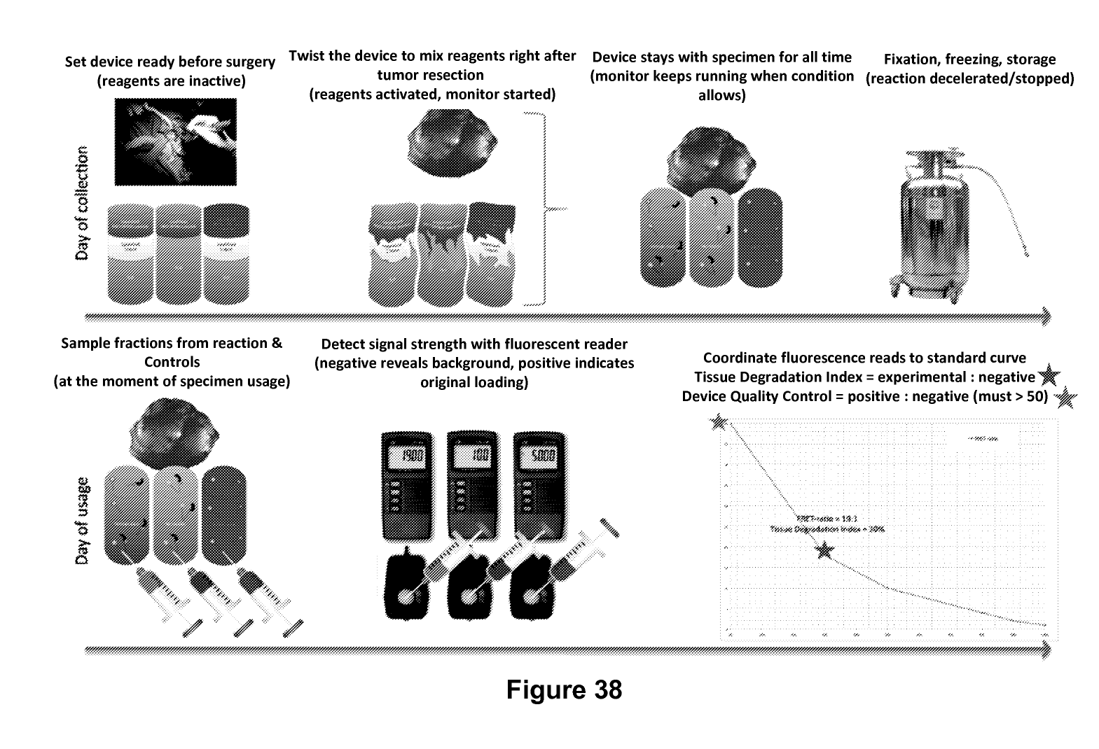

In one embodiment, a kit of the present invention includes an ambient, or extrinsic, PAV monitor, which provides an important parameter for specimen degradation measurement. In a preferred embodiment, the indicator of the extrinsic PAV is the measurement of calpain-mediate cleavage of spectrin or spectrin peptide fragments. The extrinsic PAV monitor correlates the quantity of the cleavage of spectrin with the impact of ambient PAVs, such as temperature. FIG. 38 shows an exemplary workflow using exogenic PAV monitor to assess tissue quality In one embodiment, the extrinsic PAV is adhered to a biospecimen at the time of resection, and remains with the biospecimen until diagnostic testing. This method allows for a non-invasive measurement of tissue degradation as related to ambient PAVs. In one embodiment, the extrinsic PAV consists of capsules of lyophilized calpain, labeled spectrin or spectrin fragments, and water, wherein the contents of the capsules are mixed immediately prior to storage of the biospecimen (FIGS. 38-39). In one embodiment, spectrin or spectrin fragments are fluorescently labeled. In another embodiment, the fluorescently-labeled spectrin or spectrin fragments are detected using FRET techniques. As shown in FIG. 37, tissue quality assessment peptides are used with FRET technology to examine the effect of ambient PAVs on tissue quality.

In another embodiment, a kit of the present invention includes an intrinsic, or in situ, PAV monitor. Unlike the extrinsic PAV monitor, the intrinsic PAV monitor is dependent upon direct tissue damage and functions even when biospecimens undergo chemical fixation and histological sectioning. In a preferred embodiment, the indicator of the intrinsic PAV is the measurement of calpain-mediate cleavage of spectrin or spectrin peptide fragments. As described further elsewhere herein and set forth below in the Examples, peptides are manually injected into the intercellular space of the biospecimen at the time of resection, and the in situ calpain-mediated cleavage of spectrin is monitored prior to diagnostic testing to establish the quality of the biospecimen (FIG. 13B). In one embodiment, the kit for the PAV monitor includes a mixture of lyophilized peptides (FIG. 13) prefilled into a syringe and supplied with an ampule H₂O. The water is drawn into the syringe, the contents mixed, and the contents are injected into the biospecimen. In one embodiment, the peptides are fluorescently labeled. In another embodiment, the fluorescently-labeled peptides are detected using FRET techniques.

In a preferred embodiment, a kit of the present invention includes both the intrinsic PAV monitor and the extrinsic PAV monitor for use with the same biospecimen. The combined usage of the two PAV monitors provides a more accurate and comprehensive degradation assessment of the biospecimen.

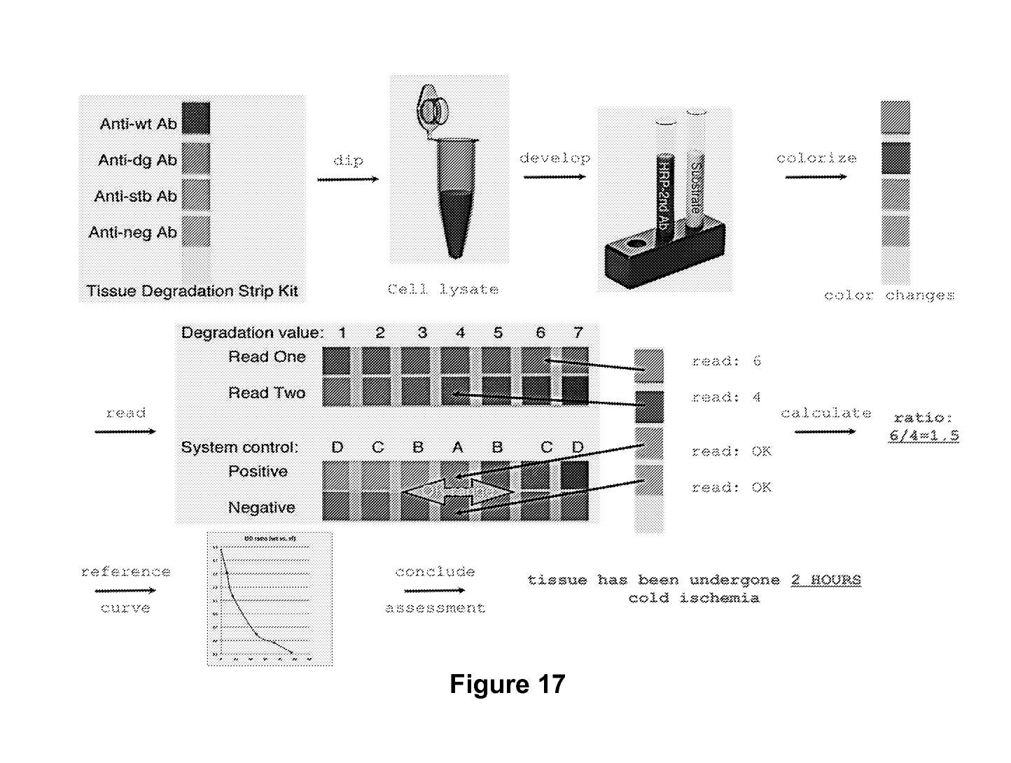

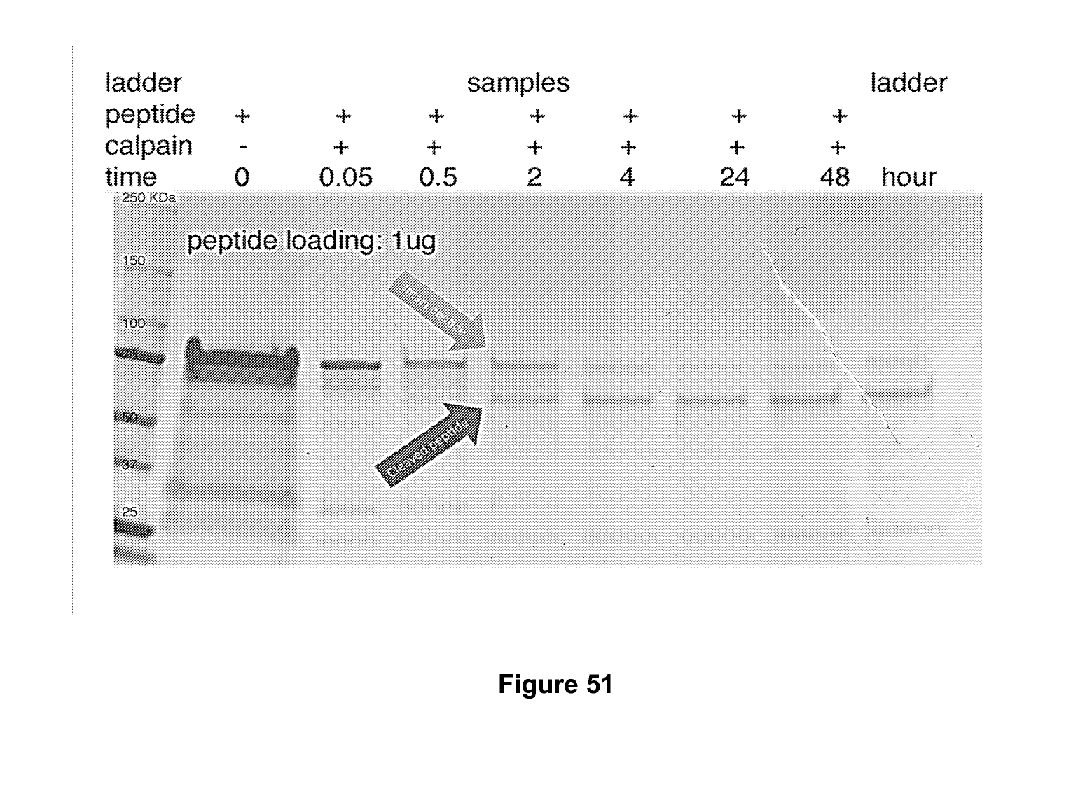

In another embodiment, a kit of the present invention includes an antibody-array based paper indicator strip for assessing the quality of a biospecimen (FIG. 17 and FIG. 51). Upon development, the strip exhibits color changes denoting a degradation value, allowing one to calculate the ratio of the intact biomarker to the cleaved form of the biomarker. The ratio is compared to the biomarker reference curve to determine the quality of the biospecimen.

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

### Example 1

### Intrinsic Indicators for Specimen Degradation

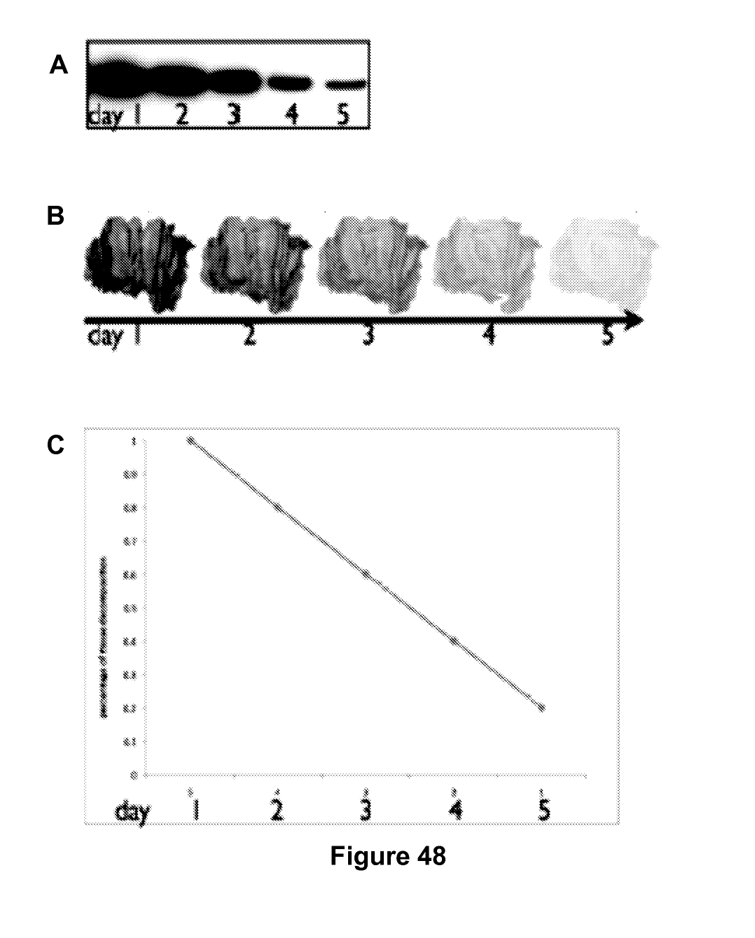

With this study, an objective standard for tissue degradation measurement was developed. It was determined whether degradation-sensitive markers could be identified via comparative proteomic analysis using experimental specimen cold-ischemic degradation models. As shown in FIG. 48, tissue decomposition may be a time-dependent event, and that during tissue decomposition a protein undergoes time-dependent changes. Degradation-sensitive proteins can serve as tissue degradation indicators, which allow for molecular integrity assessment of clinical tissue specimens prior to or simultaneously with diagnostic testing.

The materials and methods employed in these experiments are now described.

### Materials and Methods

### Cell Lines

Four commercially available human cell lines: 293T/17, MDA-MB-231 (HTB-26™), Jurkat (TIB-152™) and LNCap (CRL-1740™), were acquired from American Type Culture Collection (ATCC, Manassas, Va.). Above cell lines are of kidney, mammary gland, lymphocyte and prostate origins, respectively. Cell lines were maintained according to ATCC's recommendations at 37° C., 5% CO2, in DMEM supplemented with 10% heat-inactivated FCS, 50 units/mL penicillin, 50 ng/mL streptomycin, and 1% L-glutamine. Cells that had undergone five or fewer passages were used. Cell lines were grown until ˜80% confluent and trypsinized. Trypsinized cells were centrifuged, washed three times in ice-cold PBS (pH 7.4), and counted with a hemocytometer. Equal amounts of washed cells were assigned to tissue degradation model.

### Clinical Materials

Tissues specimens were collected by IRB-approved Yale Pathology Tissue Services. For a proof of concept study, homogeneous tumors/tissues were collected including five intracranial meningiomas, one intracranial glioma, five uterine leiomyomas, two non-tumor kidney tissues, and two non-tumor liver tissues. Multiple organs (cerebrum, cerebellum, lung, liver, kidney, heart, spleen, small intestines, large intestines, prostate, uterus, and skeletal muscle) from two patients with 13 and 15 hours post mortem interval (PMI), and cerebral middle frontal gyms in the brain from 25 patients with varied PMI (7-38 hours) were collected at autopsy. As counterparts of above tissues in other species, multiple organs from a mouse with C57BL/6J genetic background were collected. Organ and tissue diagnoses were verified by histological examination.

### Cold Ischemic Time (CIT)—Dependent Tissue Degradation Model

Harvested cells were divided into aliquots, while freshly-obtained tissues were voxelized into fractions of similar size. One aliquot/fraction of each subject was immediately snap-frozen, stored in liquid nitrogen, and assigned as time zero. The remaining aliquots/fractions were exposed to 23 Celsius in a Petri dish to mimic clinical Cold Ischemic Time (CIT) and sequentially snap-frozen at assigned CIT intervals. For cells used for proteomic analysis, the assigned CITs were 0, 0.5, 4, 8, 24, 48 hours; for tissues used for validation studies, the assigned CITs varied (0 120 hours) depending on how many fractions an individual specimen allowed to be divided into. For validation studies with formalin-fixed paraffin-embedded (FFPE) specimen, tissue fractions were fixed in neutral balanced 4% paraformaldehyde for 12 hours immediately after completion of the CIT course, followed by standard paraffin embedding.

### Cell Lysis and Protein Preparation

For proteomic analysis, frozen cells were treated with lysis buffer (7 M urea, 2 M thiourea, 4% CHAPS, 30 mM Tris, 5 mM magnesium acetate, pH 8.5) to reach a final protein concentration between 5 to 10 μg/μl. The lysates were incubated on ice for 0.5 hour with occasional vortexing. For Western blotting and other immuno-assays, cells/tissues were homogenized with a THQ-handheld homogenizer (Omni International, GA) at 15,000 rpm for 10 seconds in RIPA cell lysis buffer with protease inhibitor (Cocktail, Piece, Ill.), followed by ice bathing for 0.5 hour with occasional vortexing. All lysates were centrifuged at 13,000 g at 4° C. for 10 minutes, and supernatants were evaluated; for proteomic analysis, proteins were purified with 2D Clean-up Kit and quantified with 2D Quant Kit (GE Healthcare, NJ); for other usages, protein concentrations were directly measured with Bradford approach using Protein Assay (Bio-Rad, CA).

### Proteomic Comparison Between Specimens of Different CITs

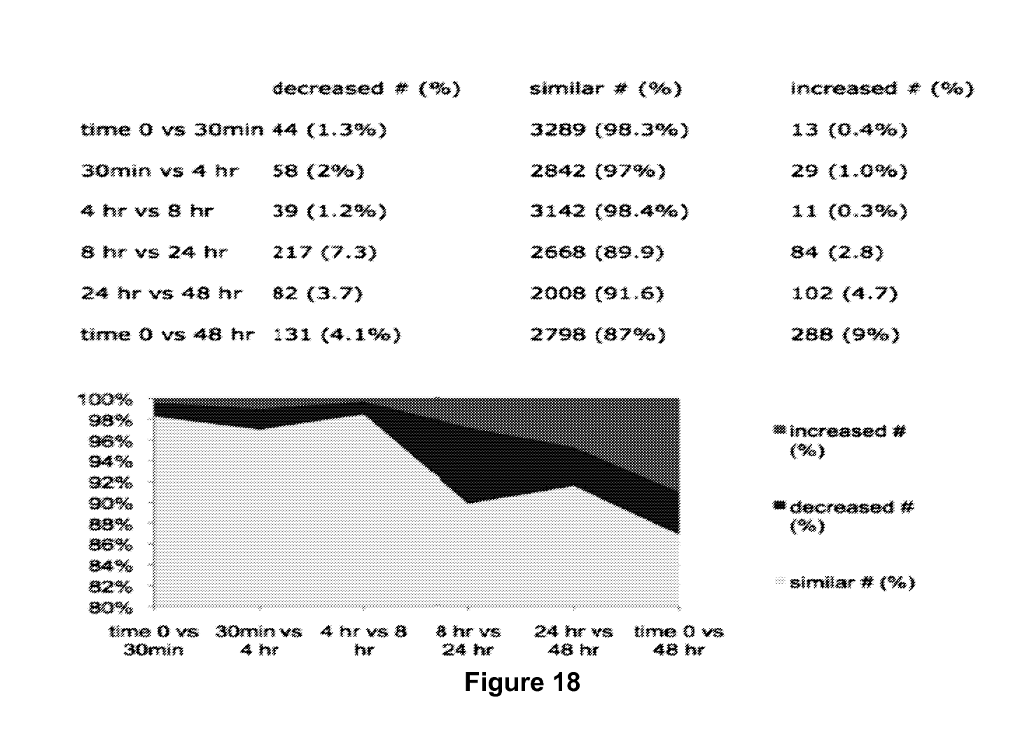

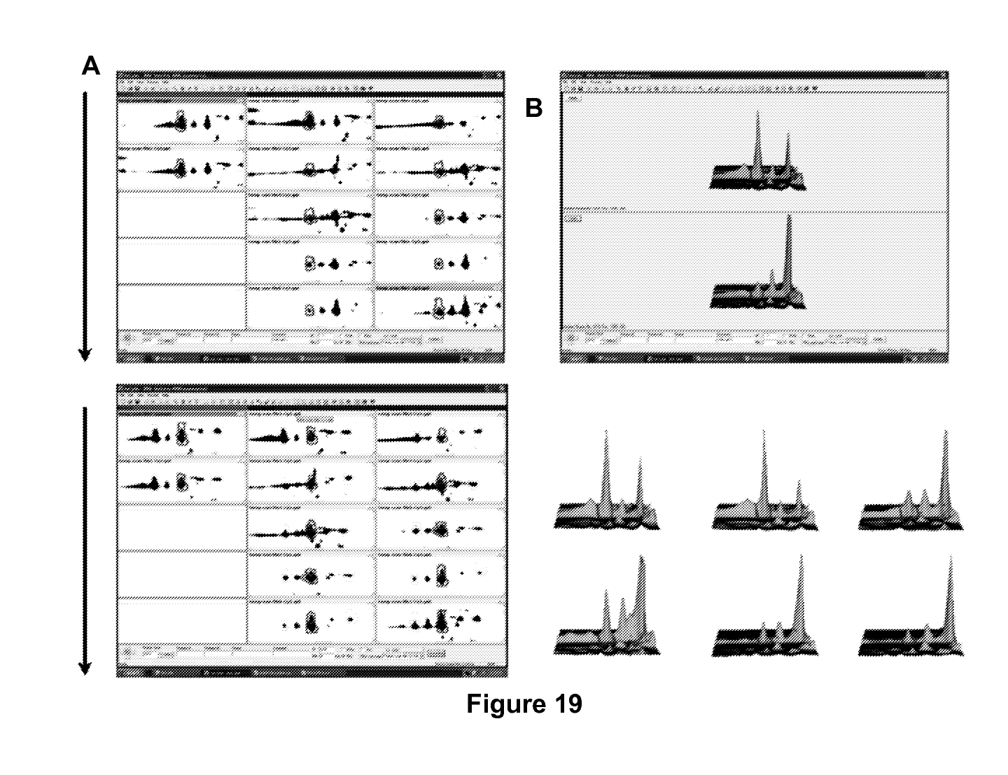

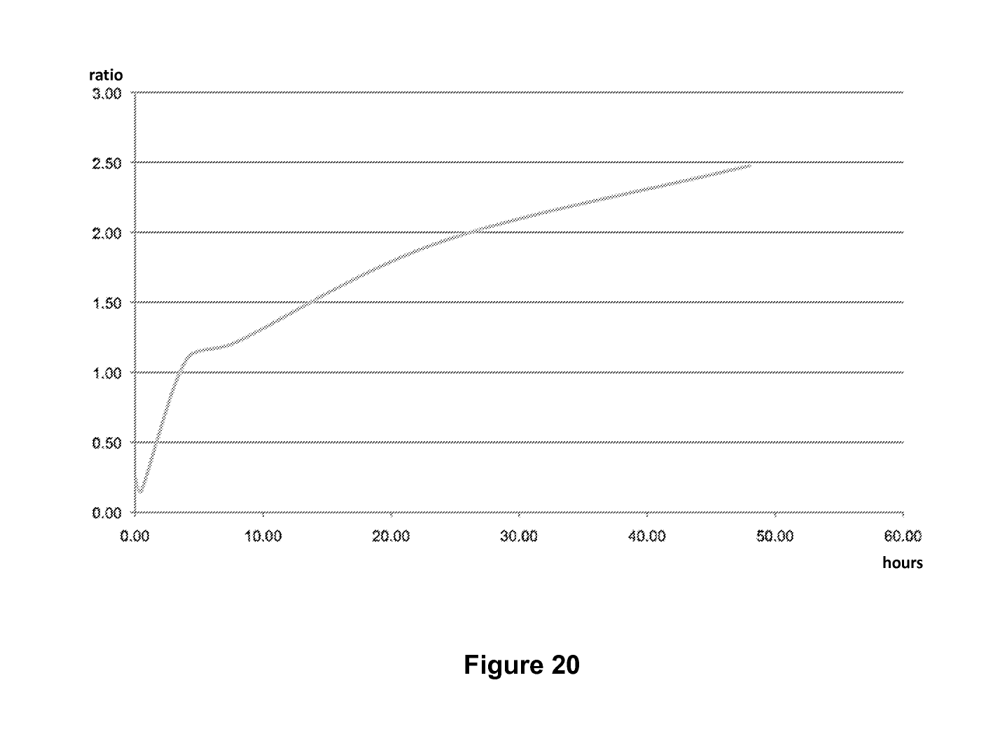

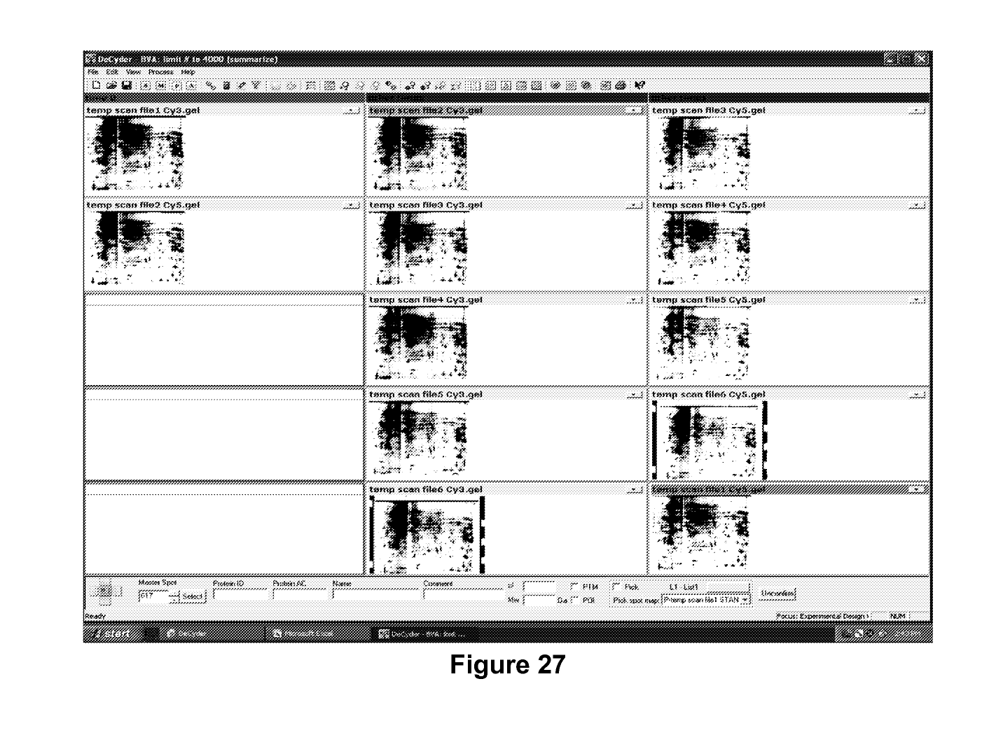

2D Fluorescence Difference Gel Electrophoresis (2D DIGE) was used for proteomic profiling following GE Healthcare's instruction (FIG. 18). In brief, 50 mg protein from each experimental group were pre-labeled with both Cy3 and Cy5 (2 repeats for each experiment) using Minimal CyDyes Kit (GE Healthcare, NJ). In addition, an internal standard was prepared by mixing 25 μg of each sample and pre-labeled with Cy2. A schematic gel design is shown in Table 1. 50 mg protein of two different groups, together with 50 mg internal standard, were separated on one large format IPG gel stripes (24 cm, pH 3-10) using Ettan IPGphor3 platform (GE Healthcare, NJ) for isoelectric focusing. Two-dimensional separation was performed on 12.5% SDS PAGE gels using Ettan DALTtwelve System (GE Healthcare, NJ). Protein spots were visualized with Typhoon 9410 Imager (GE Healthcare, NJ) and fixed afterwards (with 7.5% acetic acid 10% ethanol). FIG. 27 shows an exemplary 2D-DIGE proteomic profiling image. The spectrometric density of spots was analyzed with DeCyder software (GE Healthcare, NJ) using automated BVA mode, which assigns a quantitative (numeric) value for each protein spot after normalization with its counterpart in the universal control and creates a statistical value using reads from repeats with build-in ANOVA tools (FIG. 19). FIG. 20 depicts a representative picture of the “ratio” in 2D-DIGE analysis. The quantitative ratio between two protein spots (from FIG. 19) correlates with the time-depended decay. Protein spots, which satisfied the following conditions, were selected as candidates for protein sequencing: (1) the quantitative value was continuously increasing or decreasing throughout all observed time points; (2) the value was more than 1.5 fold different from the reads of the same spot at neighboring time points with statistical differences (P<0.05); (3) the value was in the highest 20% range among all detected protein spots at Time 0 (therefore, likely representing a “housekeeping protein”/“universal degradation indicator”); and (4) the tendency of change of this value was consistent in all four observed cell lines (reinforcing the “housekeeping” concept). One large protein spot, which had constant values across the entire observation period, was also selected for sequencing to serve as internal control for identified tissue degradation indicators.

TABLE 1
Schematic sample distribution of one cell line in 2D DIGE

| Gel | Sample 1 (Cy3 | | Sample 3 (Cy5 |
|---|---|---|---|
| number | labeling) | Sample 2 (Cy2 labeling) | labeling) |
| 1 | Time 0 hour | Master mix of all samples | Time 48 hour |
| 2 | Time 0.5 hour | Master mix of all samples | Time 0 hour |
| 3 | Time 4 hour | Master mix of all samples | Time 0.5 hour |
| 4 | Time 8 hour | Master mix of all samples | Time 4 hour |
| 5 | Time 24 hour | Master mix of all samples | Time 8 hour |
| 6 | Time 48 hour | Master mix of all samples | Time 24 hour |

MALDI-T of/T of MS and LC/MS/MS Protein Sequencing

The protein candidate list was exported from DeCyder analytical software and directly transported to the robotic Ettan Spot Picker (GE Healthcare, NJ). The picker automatically picked up candidate spots from two analytical gels and generated two sets of gel pieces for sequencing using two independent approaches. One set of gel pieces was digested with trypsin using automated Ettan TA Digester (GE Healthcare, NJ), followed by MALDI-T of/T of MS Analysis. The instrumentation used was the Applied Biosystems (CA) Model 4800 MALDI-T of/T of mass spectrometer. In brief, 80% of the digest was loaded onto the MALDI target plate using 3 mg/ml alpha-cyano-4-hydroxycinnamic acid in 0.1% trifluoroacetic acid, 50% acetonitrile as the matrix, plus an internal calibrant (1 fmol of bradykinin; protonated, monoisotopic mass=1060.569). Reflectron MS analysis summed 1250 laser shots to generate the peptide fingerprint map (PFM), and the spectra were internally calibrated using the bradykinin internal standard. Masses were chosen by the Applied Biosystems (CA) 4000 Series Explorer software (version 3.0) for MS/MS acquisition. An exclude list was used to eliminate the internal standards, and normal trypsin autolysis fragments from MS acquisition. MALDI-MS spectra were used for identifying proteins.

In parallel, another set of gel pieces were trypsin-digested and acidified with 1% acetic acid and dried in a speed-vacuum to reduce the volume to 20 ul for direct loading for LC-MS/MS analysis: LC was performed on an Agilent 1100 Nano-flow system. Mobile phase A was 94.5% MilliQ water, 5% acetonitrile, 0.5% acetic acid. Mobile phase B was 80% acetonitrile, 19.5% MilliQ water, 0.5% acetic acid. The 120 min LC gradient ran from 5% A to 35% B over 90 min, with the remaining time used for sample loading and column regeneration. Samples were loaded to a 2 cm×100 um I.D. trap column positioned on an actuated valve (Rheodyne, Wash.). The column was 13 cm×100 um I.D. fused silica with a pulled tip emitter. Both trap and analytical columns were packed with 3.5 um C18 resin (Zorbax SB, Agilent Technologies, CA). The LC was interfaced to a dual pressure linear ion trap mass spectrometer (LTQ Velos, Thermo Fisher, Mass.) via nano-electrospray ionization. An electrospray voltage of 1.8 kV was applied to a pre-column tee. The mass spectrometer was programmed to acquire, by data-dependent acquisition, tandem mass spectra from the top 15 ions in the full scan from 400-1400 m/z. Dynamic exclusion was set to 30 seconds. Mass spectrometer RAW data files were converted to MGF format using msconvert. Briefly, all searches required strict tryptic cleavage, 0 or 1 missed cleavages, fixed modification of cysteine alkylation, variable modification of methionine oxidation and expectation value scores of 0.01 or lower. MGF files were searched using X!Hunter1 against the latest library available on the GPM2 at the time.

Other searches used the cRAP contaminant library from the GPM and libraries constructed from the latest Ensemb1 release available at the time. MGF files were searched using X!!Tandem3,4 using both the native and k-scores scoring algorithms and by OMSSA6. All searches were performed using the Proteome Cluster interface. Proteins were required to have 2 or more unique peptides with E-value scores of 0.01 or less. Protein clusters were generated using the Search Tool for the Retrieval of Interacting Genes (STRING) database resource via its web portal (http://stringDb.org), which is a database of known and predicted protein interactions (developed at The Novo Nordisk Foundation Center for Protein and Research Faculty of Health Sciences at University of Copenhagen, European Molecular Biology Laboratory, The SIB Swiss Institute of Bioinformatics, Technique University Dresden, and University of Zurich). The interactions included direct (physical) and indirect (functional) associations and they were derived from genomic context, high-throughput experiments, co-expression, and previous knowledge (PubMed).

### Validation of Proteomic Findings Using Antibody-Based Immuno-Assays

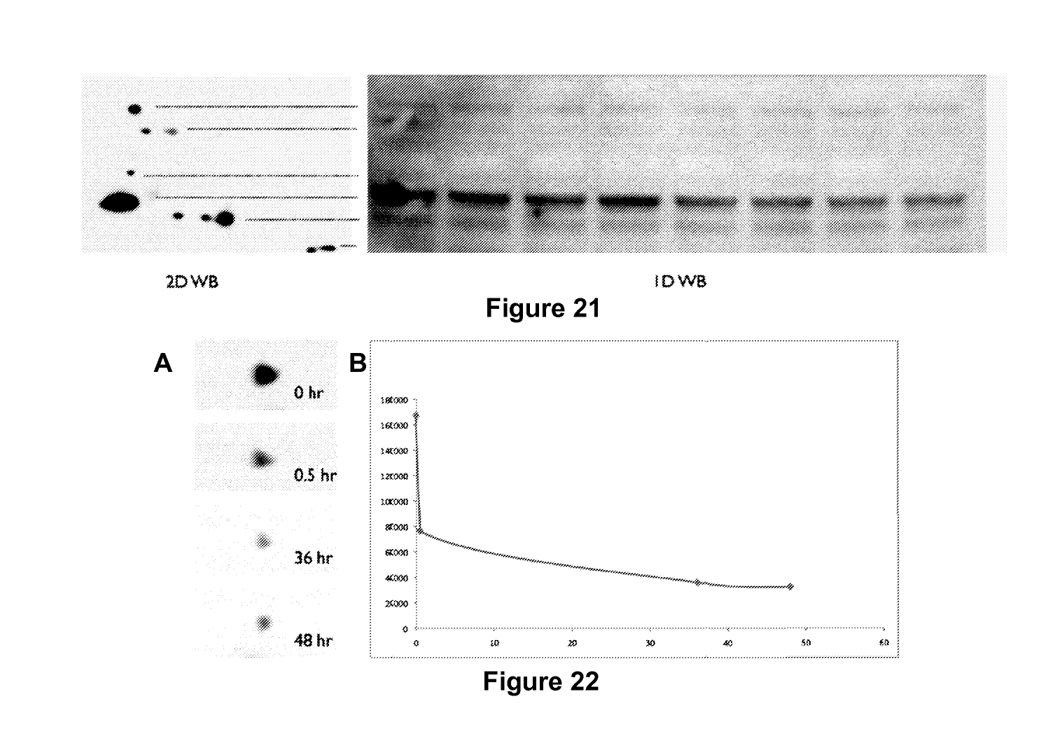

Duplicated mass spectrometry-based protein sequencing generated two independent data sets. Proteins with consensus IDs from both MALDI and LC approaches were selected for antibody-based validation studies using One Dimensional (1D) and Two Dimensional (2D) Western blotting following standard procedures (FIG. 21). Briefly, ReadyStrip™ IPG Strip (pH 4-7), mini-Protean-TGX gel (12%), and 0.2 μm nitrocellulose membrane sandwich were obtained from Bio-Rad and used for protein separation and solid-phase transfer. To minimize inter-run variations, experimental apparatuses with multiplex capacity such as mini-Protean3 Dodeca cell (Bio-Rad, CA) and TE77PWR semi-dry gel transfer system (GE Healthcare, NJ) were selected for this study. Proteins used for validation were obtained from all included tissue degradation models as aforementioned.

Antibodies were obtained from the following sources: mouse monoclonal anti-AHNAK (1:500), anti-alpha fodrin (D8B7, 1:1000), anti-actin (1:400), anti-desmoplakin I+II (1:100), rabbit monoclonal anti-alpha fodrin (EPR3017, 1:2000), anti-EEF2-C-terminal (1:10000), rabbit polyclonal anti-EEF2 (phosphor T56) (1:300), and anti-vimentin (1:1000) were obtained from Abcam (MA). Rabbit monoclonal anti-p53 (1:1000), rabbit polyclonal anti-cleaved alpha fodrin (Asp1185) (1:1000), anti-NPM (1:1000), and anti-UBE1a (1:1000) were obtained from Cell Signaling Technology (MA). Mouse monoclonal anti-PSMC3 (1:500), anti-PEX19 (1:1000), rabbit polyclonal anti-STRAP (AB1, 1:2000), anti-EIF3I (1:500), anti-SFRS1, antiDDAH1 (1:1000), anti-HSPB1 (AB-82) (1:1000), anti-UBA1 (N-terminal) (1:1000), antiDCTN2 (1:1000), and goat polyclonal anti-PPP2CA/PPP2CB (1:2500) were obtained from Sigma-Aldrich (MO). Goat polyclonal anti-tropomyosin (E-16, 1:200), anti-hnRNPC1/C2 (N-16, 1:200), anti-tropomyosin (E-16, 1:200), and anti-NACA (N-14, 1:200) were obtained from Santa Cruz Biotechnology (CA). Mouse monoclonal anti-B23 (1:1000) was obtained from Millipore and mouse monoclonal anti-EGFR (Ab-10, 1:250) was obtained from Thermo Scientific (MA).

Generation of Tissue Degradation Reference Curve Using Data from Antibody-Based Immuno-Assays

Independent of MS findings, antibody-based immuno-assays created new datasets for application of identified indicators to tissue degradation assessment: correlating antibody-revealed quantitative change of degradation indicators and specimen-experienced CIT exposure, we were able to generate tissue degradation reference curves for individual indicators. The quantity of protein was measured by using the densitometry data of its immuno-signal, i.e. signals on Western blot films. In brief, Kodak films were scanned at high resolution and numeric values of signals' strength were obtained from Adobe Photoshop's histogram (by calculating the mean value of pixels in a defined area). Signals from its degradation products or from a degradation-unaffected protein were used as internal controls. The quantitative ratio between a degradation indicator and its internal control, and its corresponding CIT strength were used to generate antibody-based tissue degradation reference curve. Microsoft Excel's built-in statistical functions were used for construction of trend lines and calculation of statistical parameters.

The results of the experiments are now described.

### Tissue Degradation Frequently Occurs Before Surgical Specimens Reach Appropriate Storage Condition

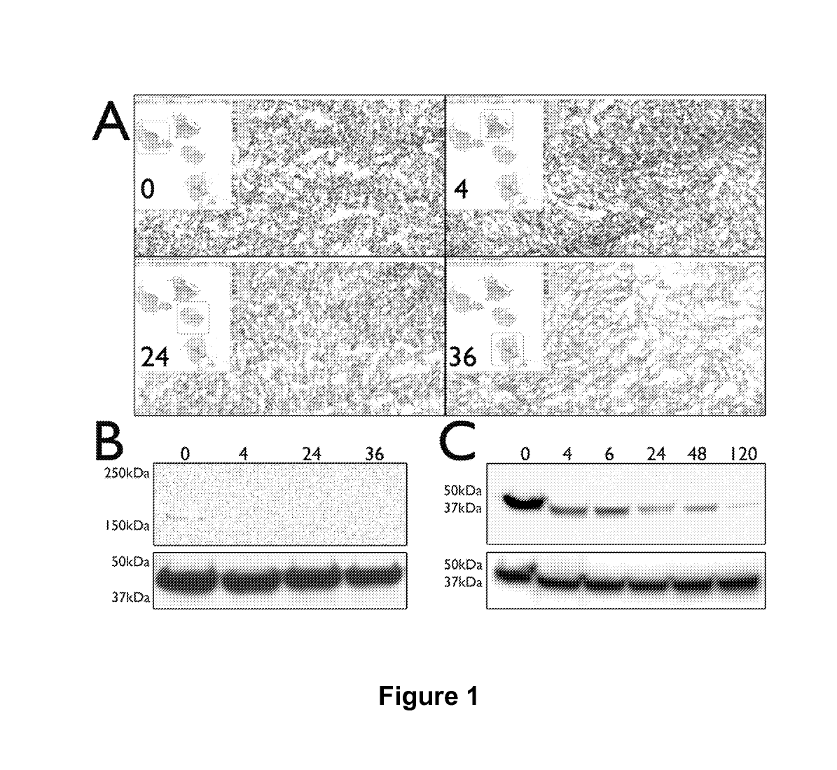

Variable degrees of degradation occur in human specimens during the interval from surgical resection to chemical fixation or snap-freezing. Herein additional exemplary evidence is provided to illustrate that tissue degradation affects the diagnostic features in specimens used for this study. By histology, significant morphological changes occur during tissue degradation, including altered intensity of nuclear staining and loss of well defined cell borders (FIG. 1A). To observe the impact of tissue degradation on commonly used diagnostic markers, the alteration of EGFR and p53 was examined within tissues that underwent CIT-dependent degradation was investigated. Data shows that EGFR completely degraded by 4 hours of CIT exposure in a meningioma specimen (FIG. 1B). Dynamic degradation of p53 was also observed in cells that underwent CIT-dependent degradation (FIG. 1C).

Cell Degradation Levels Quantitatively Correlate with the Degradation Status of a Subset of Proteins

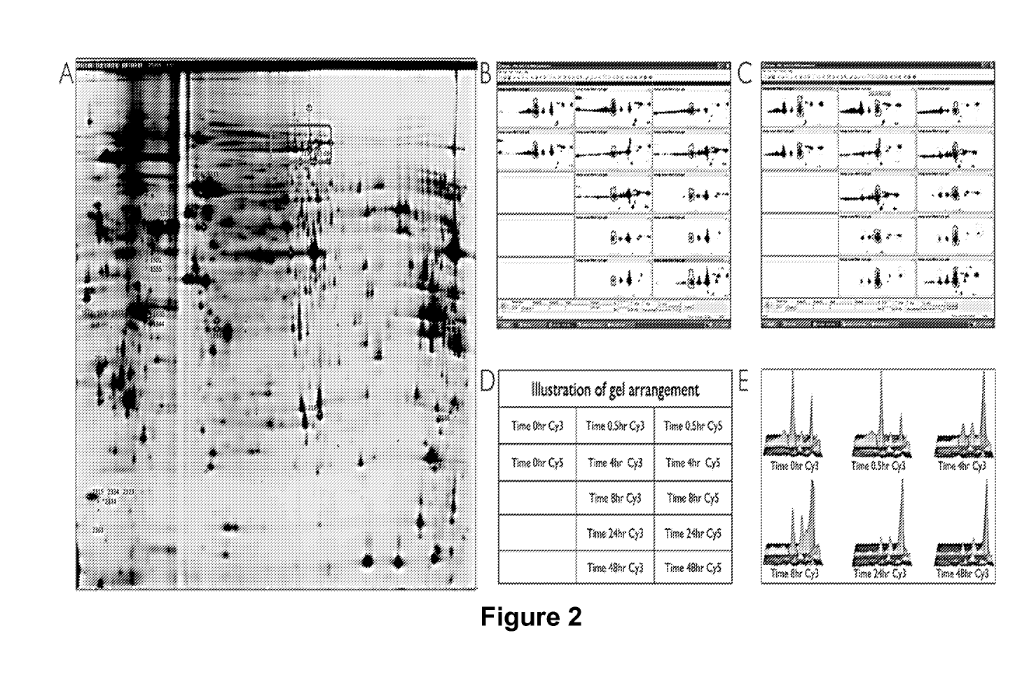

To observe the overall cellular response to degradation, four cell lines were assigned to a CIT-dependent tissue degradation experimental model and utilized a 2D DIGE-based proteomic approach to determine protein changes between cells at different CIT exposure (FIG. 2A). For each cell line, six levels of CIT exposure were conducted and evaluated on 12 2D gel images (results were performed in duplicate).

Individual proteins on 2D gels were quantitatively analyzed with DeCyder software. While in average 86.9% of 2798 protein spots showed no significant (1.5 fold) quantitative changes during 48 hours of CIT exposure, protein spots of interest were identified according to the following criteria: the protein was significantly expressed (among 20% of proteins with highest expression intensity) at each time point in all four cell lines and the protein revealed either continuous quantitative increase or continuous quantitative decrease at consecutive time points. Fulfilling this criteria, a total of 50 protein spots had a continuous loss or increase during CIT in all cell lines (FIG. 2B-E). These data indicate that protein changes occur during the cell degradation process and some of these proteins changes correlate quantitatively with the impact of CIT, i.e. they correlate with the stage of cell degradation. Therefore, by measuring the degradation status of these proteins in tissue, the degradation level of the respective specimens could be assessed and thereby these proteins could be utilized as Tissue Degradation Indicators (TDIs).

### Identification of TDIs

Candidate protein spots from 2D gel analysis were subjected to both MALDI- and LC-based peptide sequencing. Mass spectrometric findings from both platforms are presented in Table 2. 26 proteins with continuous quantity change (TDIs) and one stable protein without any quantity change (actin) which can be used for control purposes were successfully sequenced by both platforms. Of these proteins, 19 had shown continuous quantity loss during tissue degradation, while seven had shown continuous quantity accumulation; an additional prominent spot that had remained quantitatively stable at all time points was selected for control purposes (#20, beta-actin, ACTB). In six instances, two or more separate protein spots on 2D gels were identified as parts of the same protein (#1 TDI: AHNAK, nucleoprotein; #2 TDI: SPTAN1, alpha II spectrin; #3 TDI: EEF2, eukaryotic translation elongation factor 2, #8 TDI: EIF3I, eukaryotic translation initiation factor 3; #17 TDI: TPM3, tropomyosin 3; and #19 TDI: HSPB1, heat shock 27 kDa protein 1), and different fragments of the same protein sometimes revealed reciprocal intensity changes with increasing CIT. Reciprocal intensity changes were observed in protein parts of either different isoelectric point (e.g. #3 EEF2) or different molecular size (e.g. #2 alpha II spectrin) at consecutive time points. Computerized STRING analysis revealed the protein cohort of TDI candidates to mainly function as protein binding elements and/or structural constituents of the cytoskeleton (statistical significance P<0.01).

TABLE 2
Summary of protein IDs identified via mass spectrometry.

| | | Unique | Total | MW | | | Brief protein |
|---|---|---|---|---|---|---|---|
| # | Trend* | hits** | hits** | (kDa) | Accession # | Description | function |
| 1 | Down | 181 | 548 | 628.7 | ENSP00000367263 | AHNAK | Cell membrane |
| | | | | | | nucleoprotein | differentiation, repair, |
| | | | | | | | Calcium homeostasis |
| 2 | Down | 144 | 577 | 284.9 | ENSP00000361824 | SPTAN1, | Filamentous |
| | | | | | | Alpha-II spectrin | cytoskeletal protein, |
| | | | | | | | essential scaffold |
| | | | | | | | protein that stabilizes |
| | | | | | | | the plasma membrane |
| 3 | Down | 20 | 51 | 95.3 | ENSP00000307940 | EEF2, | GTP-dependent |
| | | | | | | eukaryotic | translocation of a |
| | | | | | | translation | growing protein chain |
| | | | | | | elongation | from the A- to P-site |
| | | | | | | factor 2 | of the ribosome |
| 4 | Down | 51 | 265 | 81.9 | ENSP00000411293 | GSN, gelsolin | Calcium-regulated |
| | | | | | | | protein, assembly and |
| | | | | | | | disassembly of actin |
| | | | | | | | filaments |
| 5 | Down | 76 | 1928 | 53.6 | ENSP00000224237 | VIM, vimentin | Intermediate |
| | | | | | | | filaments, making up |
| | | | | | | | cytoskeleton, |
| | | | | | | | maintaining cell |
| | | | | | | | shape, cytoplasm |
| | | | | | | | integrity, and |
| | | | | | | | stabilizing cytoskeletal |
| | | | | | | | interactions |
| 6 | Down | 15 | 34 | 38.4 | ENSP00000392270 | STRAP, | Regulation of cell |
| | | | | | | serine/threonine | proliferation, |
| | | | | | | kinase receptor | programmed cell |
| | | | | | | associated protein | death, differentiation |
| 7 | Down | 2 | 2 | 36.7 | ENSP00000251074 | NUP37, | Part of the Nup107- |
| | | | | | | nucleoporin | 160 subcomplex of the |
| | | | | | | 37 kDa | nuclear pore complex, |
| | | | | | | | essential for the |
| | | | | | | | transport of |
| | | | | | | | macromolecules |
| | | | | | | | between the cytoplasm |
| | | | | | | | and nucleus, |
| | | | | | | | microtubule |
| | | | | | | | attachment, mitotic |
| | | | | | | | progression, and |
| | | | | | | | chromosome |
| | | | | | | | segregation |
| 8 | Down | 4 | 4 | 36.5 | ENSP00000362688 | EIF3I, | Eukaryotic translation |
| | | | | | | eukaryotic | initiation factor, |
| | | | | | | translation | prevents the |
| | | | | | | initiation | association of small |
| | | | | | | factor 3, subunit I | and large subunits of |
| | | | | | | | ribosome until |
| | | | | | | | elongation is ready to |
| | | | | | | | begin |
| 9 | Down | 12 | 42 | 35.6 | ENSP00000418447 | PPP2CA-001, | Catalytic unit of |
| | | | | | | protein | protein phosphatase |
| | | | | | | phosphatase 2, | 2A, was used to |
| | | | | | | catalytic subunit, | associate with post |
| | | | | | | alpha isozyme | mortem interval (PMI) |
| 10 | Down | 4 | 8 | 33.5 | ENSP00000361777 | SET, SET | Participates in |
| | | | | | | nuclear oncogene | apoptosis, nucleosome |
| | | | | | | | assembly, and histone |
| | | | | | | | binding |
| 11 | Down | 5 | 7 | 32.8 | ENSP00000357051 | PEX19, | Necessary for early |
| | | | | | | peroxisomal | peroxisomal |
| | | | | | | biogenesis | biogenesis |
| | | | | | | factor 19 | |
| 12 | Down | 2 | 2 | 32.8 | ENSP00000288398 | TPM1, | Binds to actin, |
| | | | | | | tropomyosin 1 | stabilizes cytoskeleton |
| | | | | | | (alpha) | actin |
| 13 | Down | 4 | 7 | 32.6 | ENSP00000296930 | NPM1P21, | Ribosome biogenesis, |
| | | | | | | nucleophosmin | centrosome |
| | | | | | | (nucleolar | duplication, protein |
| | | | | | | phosphoprotein | chaperoning, histone |
| | | | | | | B23, numatrin) | assembly, cell |
| | | | | | | | proliferation |
| 14 | Down | 10 | 43 | 32.3 | ENSP00000319690 | HNRNPC, | Binds pre-mRNA, |
| | | | | | | heterogeneous | involving pre-mRNA |
| | | | | | | nuclear | splicing, translation |
| | | | | | | ribonucleoprotein | |
| | | | | | | C (C1/C2) | |
| 15 | Down | 2 | 3 | 32 | ENSP00000401336 | SFRS1, pre- | Ensures the accuracy |
| | | | | | | mRNA- | of splicing and |
| | | | | | | splicing factor | regulates alternative |
| | | | | | | SF2, P33 subunit | splicing |
| 16 | Down | 4 | 5 | 31.1 | ENSP00000284031 | DDAH1, | Regulation of nitric |
| | | | | | | Dimethylarginine | oxide generation, low |
| | | | | | | dimethylamino- | NO protecting tissue |
| | | | | | | hydrolase 1 | from ischemic damage |
| 17 | Down | 2 | 2 | 29 | ENSP00000357521 | TPM3, | Binds to actin, |
| | | | | | | tropomyosin 3 | stabilizes cytoskeleton |
| | | | | | | | actin |
| 18 | Down | 6 | 18 | 23.4 | ENSP00000397352 | NACA, | Protect growing |
| | | | | | | Nascent | polypeptide chains as |
| | | | | | | polypeptide- | they emerge from |
| | | | | | | associated | ribosome |
| | | | | | | complex | |
| | | | | | | subunit alpha | |
| | | | | | | (NAC-alpha) | |
| 19 | Down | 4 | 4 | 22.8 | ENSP00000248553 | HSPB1, heat | Induced by |
| | | | | | | shock 27 kDa | environmental stress |
| | | | | | | protein 1 | to participate in stress |
| | | | | | | | resistance and actin |
| | | | | | | | organization |
| 20 | Stable | 67 | 1338 | 41.7 | ENSP00000349960 | ACTB, actin, beta | Involved in cell |
| | | | | | | | motility, structure, |
| | | | | | | | integrity |
| 21 | Up | 30 | 64 | 117.8 | ENSP00000366568 | UBA1, | Catalyzes the first step |
| | | | | | | ubiquitin-like | in ubiquitin |
| | | | | | | modifier activating | conjugation to mark |
| | | | | | | enzyme 1 | cellular proteins for |
| | | | | | | | degradation; may |
| | | | | | | | function in DNA |
| | | | | | | | repair |
| 22 | Up | 13 | 32 | 53.5 | ENSP00000252934 | ATXN10, ataxin 10 | Plays role in the |
| | | | | | | | maintenance of a |
| | | | | | | | critical intracellular |
| | | | | | | | glycosylation level |
| | | | | | | | and homeostasis |
| 23 | Up | 17 | 49 | 50.1 | ENSP00000336799 | TUBA1B, | Part of microtubules; |
| | | | | | | tubulin, alpha 1b | acts as a scaffold to |
| | | | | | | | determine cell shape |
| 24 | up | 23 | 68 | 49.2 | ENSP00000298852 | PSMC3, | ATPase on 26S |
| | | | | | | proteasome | proteasome which |
| | | | | | | (prosome, | cleave peptides in an |
| | | | | | | macropain) | ATP/ubiquitin- |
| | | | | | | 26S subunit, | dependent process |
| | | | | | | ATPase, 3 | |
| 25 | Up | 15 | 33 | 48.1 | ENSP00000272227 | PDIA6, | ER resident protein; |
| | | | | | | protein disulfide | catalyzes |
| | | | | | | isomerase family | isomerization of |
| | | | | | | A, member 6 | disulfide bonds in |
| | | | | | | | proteins, inhibit |
| | | | | | | | aggregation of |
| | | | | | | | misfolded proteins |
| 26 | Up | 15 | 24 | 47.3 | ENSP00000157812 | PSMC4, | ATPase on |
| | | | | | | proteasome | proteasome that cleave |
| | | | | | | (prosome, | peptides in an |
| | | | | | | macropain) 26S | ATP/ubiquitin- |
| | | | | | | subunit, ATPase, 4 | dependent process. |
| 27 | Up | 16 | 44 | 44.8 | ENSP00000408910 | DCTN2, dynactin 2 | Binds to both |
| | | | | | | (p50) | microtubules and |
| | | | | | | | cytoplasmic dynein, |
| | | | | | | | involved in |
| | | | | | | | cellular/subcellular |
| | | | | | | | movement |

*Trend: the tendency of a protein's quantitative change during tissue degradation (compared to protein's original status at Time 0).
**Unique hits and Total hits represent the reads from mass spectrometric analysis.

### Validation of TDIs in Clinical Specimens by Western Analysis

Western blotting was selected for TDI validation using 64 clinical human specimens and 12 mouse specimens. Considering that degradation-induced protein changes may include both quantitative changes and intact/breakdown conversions (isoelectric changes and molecular weight changes), both 1D and 2D Western blotting were applied to monitor these potential alterations.

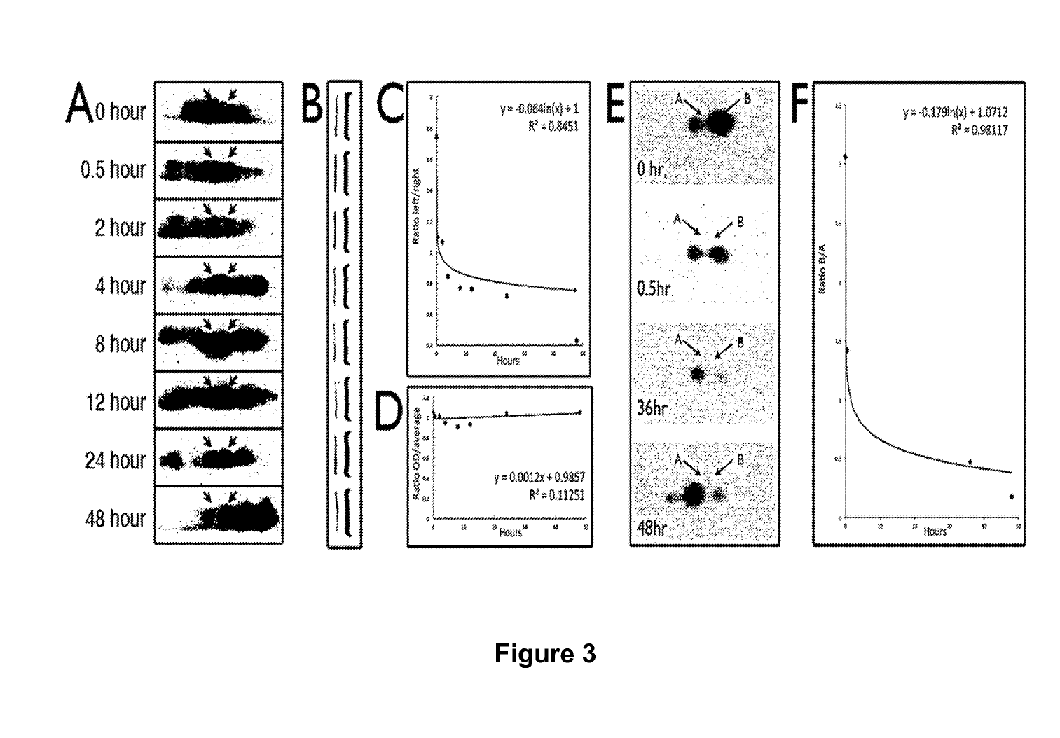

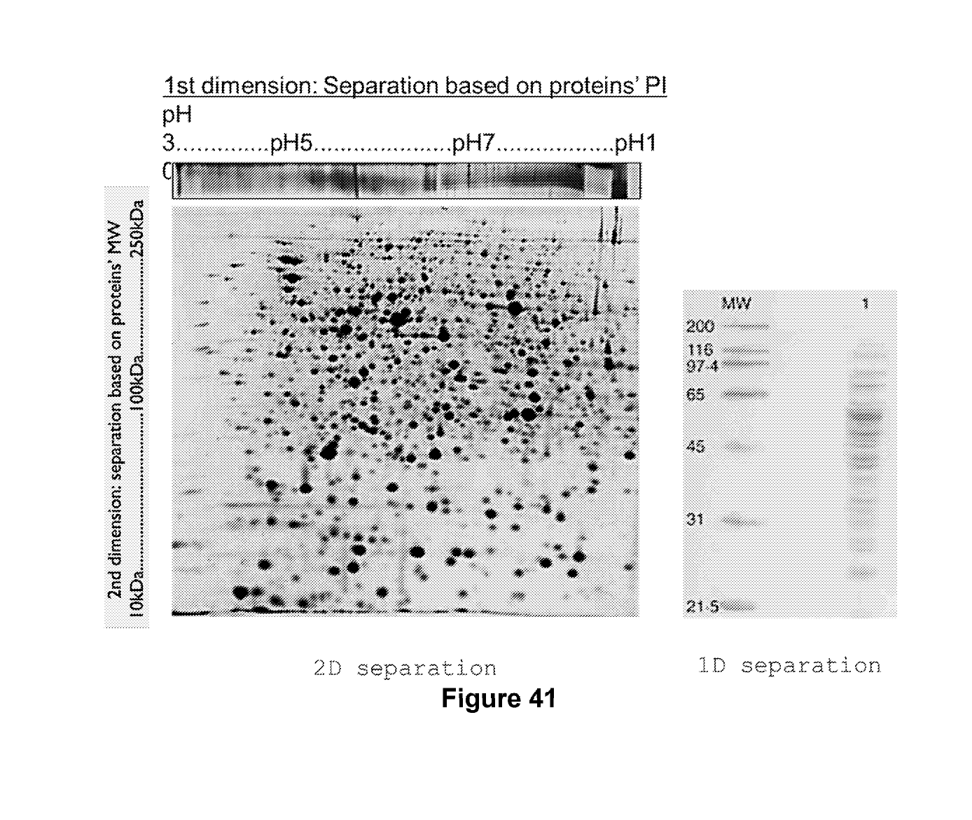

2D Western analysis successfully validated proteomic findings on isoelectric point (PI)-based TDI degradation for 11 proteins, EEF2 (#3), EIF3I (#8), PPP2C1 (#9), NPM1P21 (#13), HNRNPC (#14), SFRS1 (#15), DDAH1 (#16), TPM3 (#17), NACA (#18), PSMC3 (#24), and DCTN2 (#27) (Table 2). FIGS. 3A&C shows successful validation of degradation-induced PI changes of EEF2: 2D Western blotting exhibited a dynamic conversion from EEF2's original form to a degraded form beginning at 0.5 hour of CIT. The quantitative ratio between the two spots strongly correlates with respective CIT-impact (hours); when the same protein samples are evaluated by 1D Western analysis alone, the CIT impact on EEF2 cannot be detected (FIGS. 3B and 3D). FIG. 41 shows a comparison of 1D separation of proteins to 2D separation. The protein PPP2C1 (#9 TDI) also exhibited a dynamic conversion between its two forms with different PIs (indicated with blue and red arrows in FIG. 3E). The quantitative ratio between these forms strongly correlated with CIT impact with a R² value of 0.98 for the logarithmical curve (FIG. 3F).

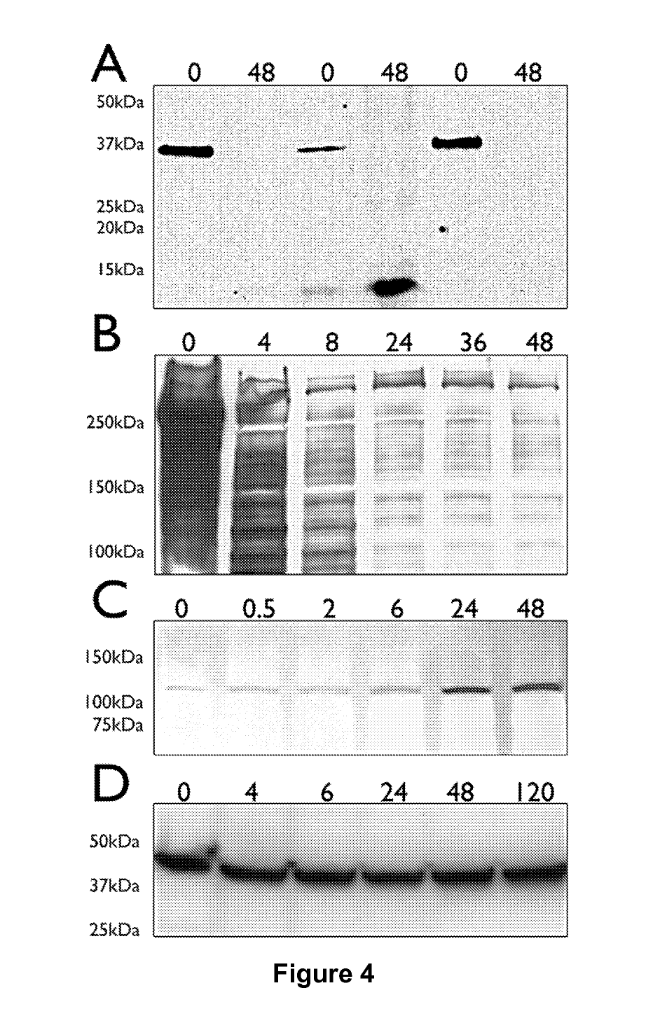

2D Western analysis, although capable of validating proteomic findings, is cumbersome and costly. Therefore, TDIs that can be validated using a simple 1D Western blotting may be more easily adapted into to a “tissue assessment tool.” The validation result using 1D Western analysis is summarized as follows: (1) upon 1D Western analysis, proteins were represented by correct molecular size, and showed expected decrease of expression intensity with increase CIT exposure. These proteins included GSN (#4), VIM (#5), SET (#10), PEX (#11), TPM1 (#12), HSPB1 (#19), B23 (#13, FIG. 4A); (2). These TDIs include AHNAK (#1, FIG. 4B), NUP37 (#7), ATXN10 (#22), and TUBA1B (#23), PDIA6 (#25), and PSMC4 (#26); (3) upon validation, proteins with quantitatively observed continuous increase revealed accumulation upon Western blotting validation (FIG. 4C); (4) beta actin (#20) had been identified as a strongly expressed protein without significant degradation-induced changes, and was successfully validated by 1D Western analysis (FIG. 4D); (5) one protein (#2, alpha II spectrin) revealed consistent quantitative decline of its native form and consistent quantitative increase of its breakdown product.

### The Quantitative Ratio of Alpha II Spectrin and its Breakdown Products: a Better Tool for Degradation Assessment

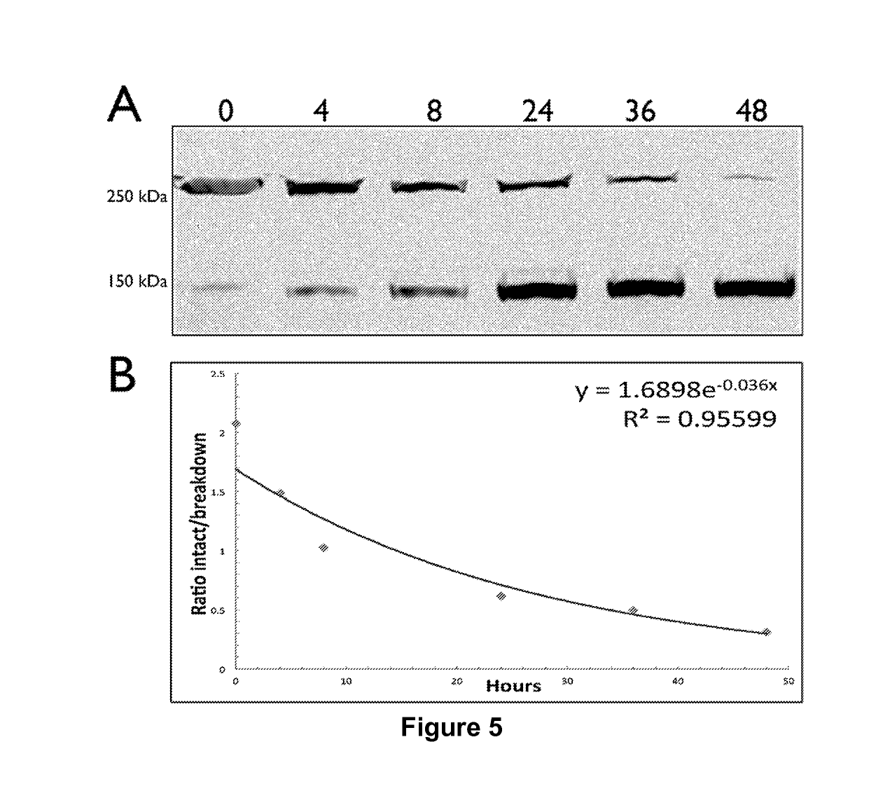

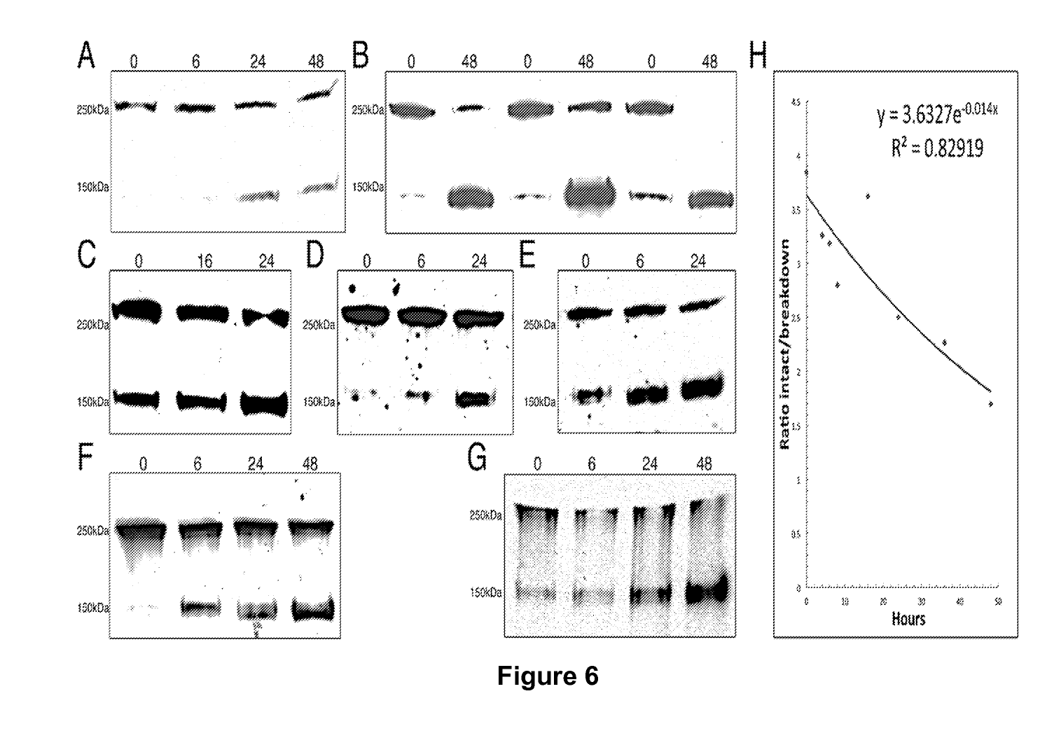

By 1D Western analysis, alpha II spectrin exhibits a continuous and dynamic conversion between its intact form and its breakdown form during the process of protein degradation. Several commercially available spectrin-antibodies as well as antibodies generated in our lab were used to simultaneously recognize intact spectrin and its breakdown products (FIGS. 5A and 5B). Native spectrin and its breakdown product revealed continuous reciprocal expression during CIT exposure in human surgical specimens (FIGS. 6A-6C) and in mouse tissues of varied types (FIGS. 6D-6G). The association between spectrin breakdown and CIT stage is demonstrated in FIG. 6H.

Exploration of Spectrin-Based Degradation Assessment with Autopsy and FFPE Tissues

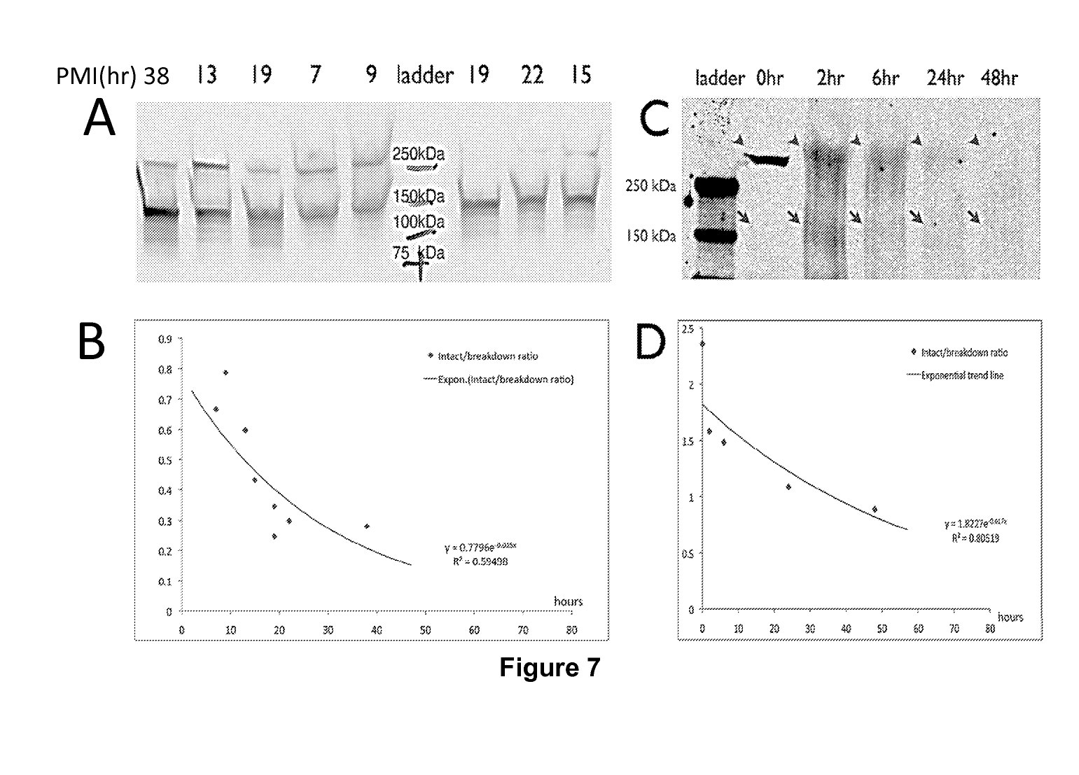

Compared to surgical specimens, autopsy specimens degrade under different microenvironments. A post mortem interval (PMI) elapses before tissue samples can be procured. Taken that spectrin breakdown is a cumulative indicator of cell damage (necrosis, apoptosis, traumatic damage, and cytotoxicity) (Takatsuka et al., 2005, Biochem Biophys Res Commun. 336:316-323; Zadran et al., 2010, J. Neurosci. 30:1086-1095; Mondello et al., 2010, J Neurotrauma 27:1203-1213; Lowy et al., 1994, J. Neurochem. 63:886-884; Duda et al., 2002, Biol. Chem. 383:785-791; Park et al., 2007, Exp Neurol. 204:49-57; Benz et al., 2008, Int Immunopharmacol. 8:319-324; Hwang et al., 2008, Neurosci Lett. 435:251-256.; Brophy et al., 2009, J Neurotrauma. 26:471-479) the correlation between spectrin breakdown and the respective PMI in autopsy specimens was further explored. A tight association between spectrin breakdown and respective PMI of specimens was found using autopsy brain tissues (FIGS. 7A and 7B). This data highlighted the potential of TDI application to autopsy tissue quality assessment as well as to studies in forensic medicine.

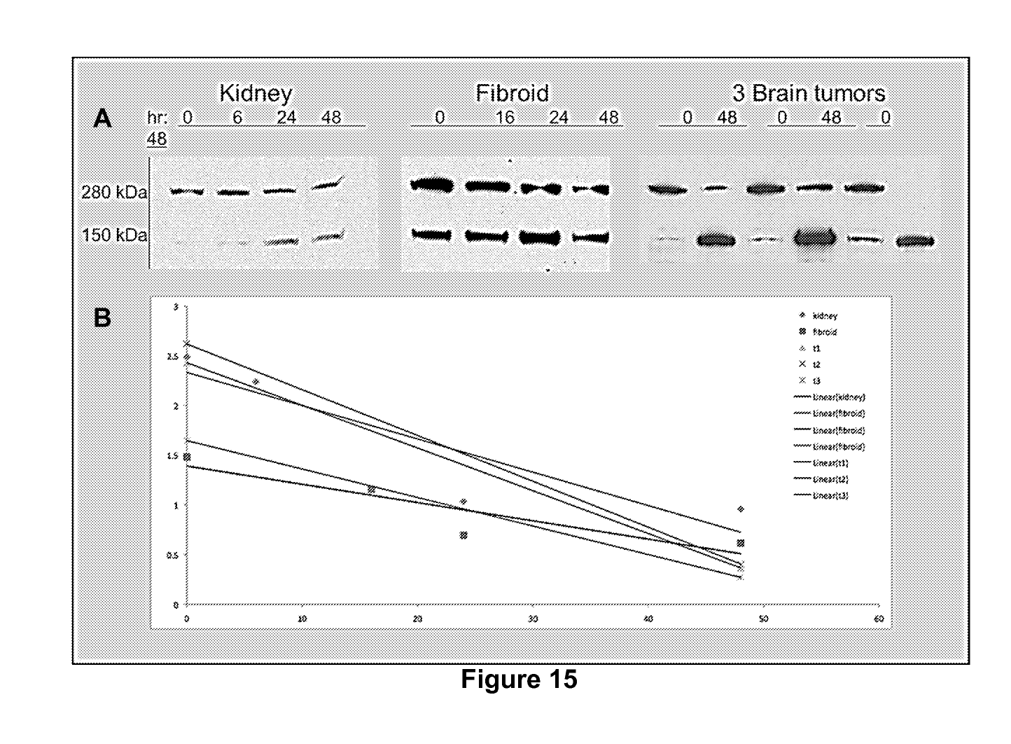

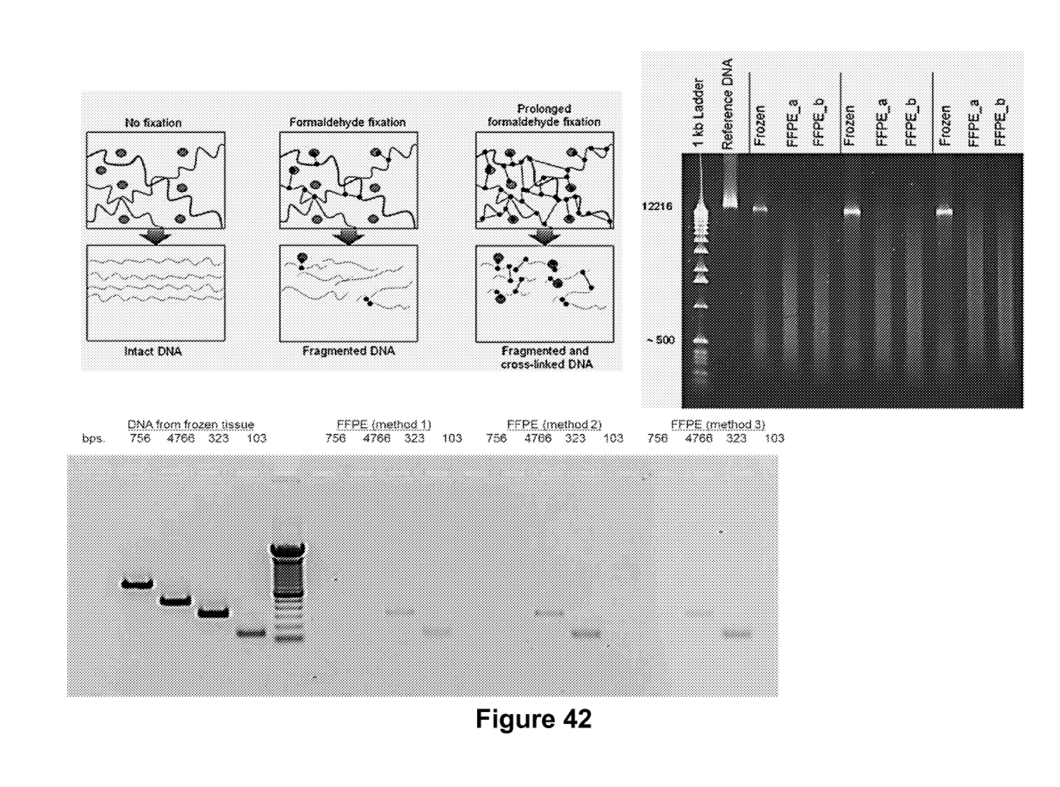

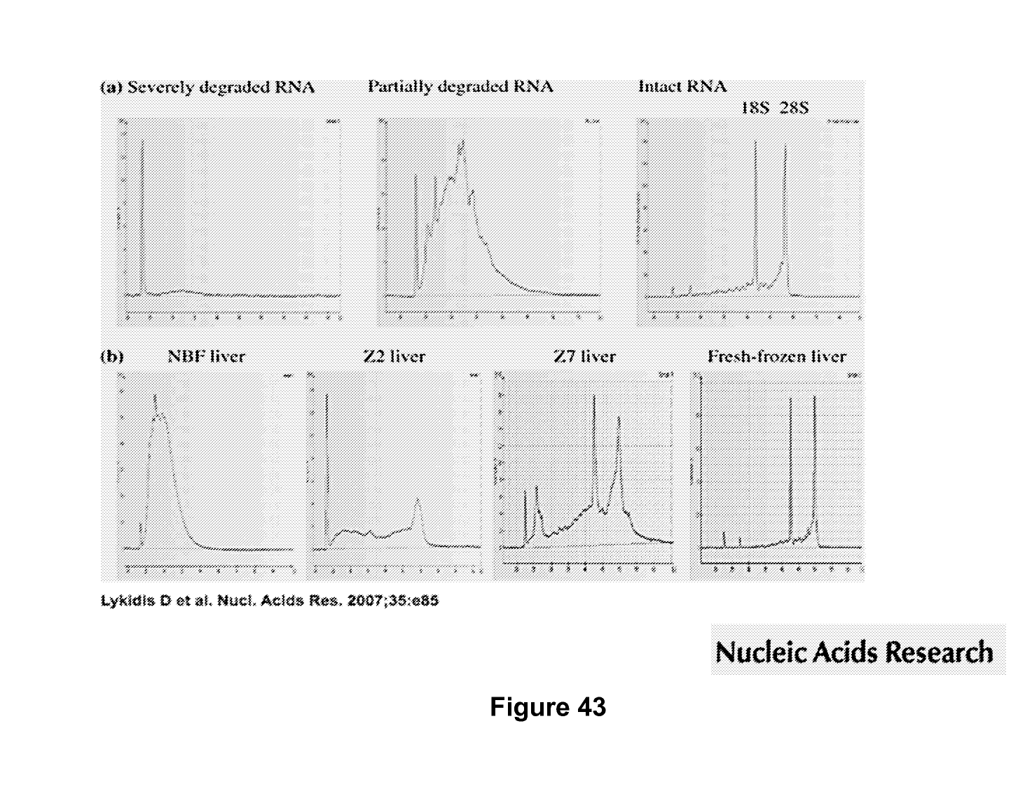

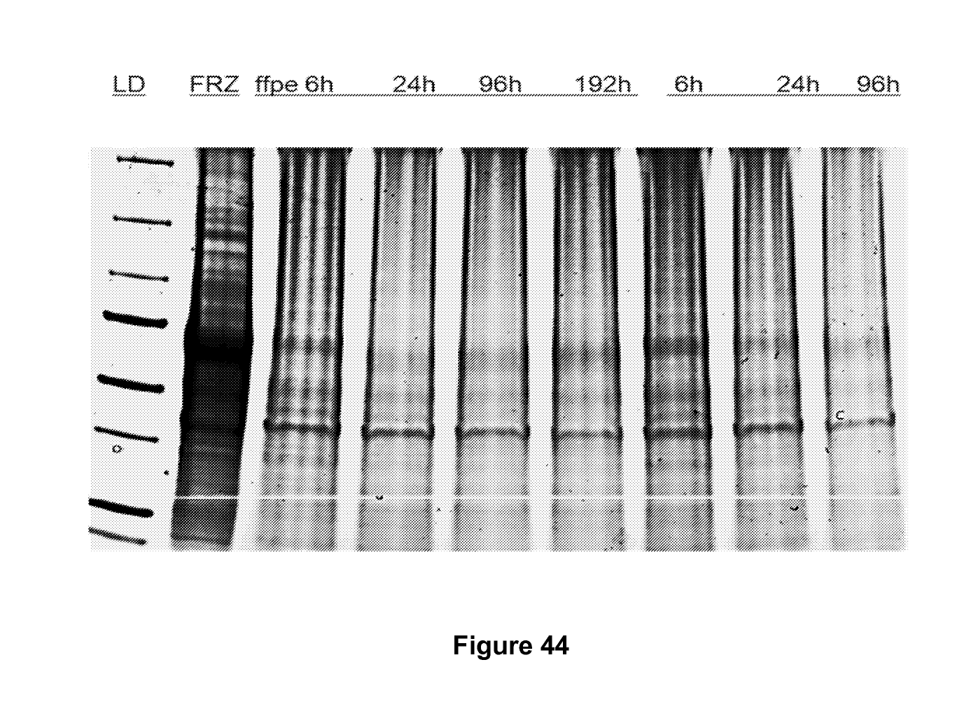

For histopathological review, formalin fixation with subsequent embedding into paraffin is the most widely used method. In analogy to frozen specimens, varied degrees of degradation may have occurred in formalin-fixation paraffin-embedded (FFPE) tissues prior to formalin fixation and pre-fixation degradation in FFPE specimens may affect biochemical quality of the specimens FIGS. 42-44 show a comparison of DNA, RNA, and protein quality in freshly frozen tissues to FFPE tissue. To reveal pre-fixation degradation, surgical tissues were assigned into experimental degradation models prior to formalin fixation and paraffinization. FIG. 15 depicts the use of surgical tissues to validate spectrin as a decomposition indicator. In general, the efficiency of protein extraction from FFPE tissue was low and the protein quality was poor due to formalin-induced inter- or intra-protein crosslinks affecting protein distribution in Western blot analysis. Higher molecular weight bands, such as spectrin, were displayed as vague bands (FIGS. 7C and 7D), whereas lower molecular weight proteins, such as beta-actin, remained better preserved and could still be revealed as discrete bands of acceptable quality. Although the signal of spectrin breakdown is not optimal, quantitative information about spectrin breakdown was extracted by quantifying the total signal from Western blot and thereby determining the association between spectrin breakdown and CIT impact in FFPE tissues.

As demonstrated herein, tissue degradation continues to occur in surgical pathology specimens immediately after tissue resection. Effects of tissue degradation are characterized by loss of histolological integrity and by loss of integrity of diagnostic biomolecules. Tissue degradation may alter biomolecule expression and misguide patient care in severe instances (Cross et al., 1990, J Clin Pathol. 43:597-599; Sauter et al., 2009, J Clin Oncol. 27:1323-1333; Hammond et al., 2012, J Clin Onco129:e458; Albanell et al., 2009, Clin Transl Oncol. 11:363-375; Bartlett et al., 2012, J Clin Pathol. 64:649-653; Chivukula et al., 2008, Mod Pathol. 2008 363-368; Moatamed et al., 2012, Am J Clin Pathol. 136:754-761; Selvarajan et al., 2003, Histochem Cell Biol. 120:251-255; Striebel et al., 2008, Am J Clin Pathol. 2008:383-390; Werner et al., 2000, Am J Surg Pathol. 24:1016-1019; Derecskei et al., 2006, Pathol Oncol Res. 12:243-246; van Kemenade et al., 2007, Ned Tijdschr Geneeskd. 151:1283-1286; Wolff et al., 2007, J Clin Oncol. 25:118-145; Chin et al., 2012, Can Urol Assoc J. 4:13-25. Therefore, efforts toward both tissue protection (Loi et al., 2012, Lancet Oncol. 12:1162-1168; Selvarajan et al., 2003, Histochem Cell Biol. 120:251-255; Striebel et al., 2008, Am J Clin Pathol. 2008:383-390; R^{a1} et al., 2005, Proteomics 5:3262-3277; Steiniche et al., 2008, Ugeskr Laeger170:1050; Tammen et al., 2008, Methods Mol. Biol. 428:35-42; Vanderklish et al., 2000, Proc. Natl. Acad. Sci. USA 97:2253-2258; Suzuki et al., 2005, Biochem Biophys Res Commun. 330:454-460; Cummings et al., 2002, Proc. Natl. Acad. Sci. USA. 99:6603-6606; Kisselev et al., 2006, J. Biol. Chem. 281:8582-8590) as well as tissue degradation measurement are critical to maximize confidence in sample interpretation.

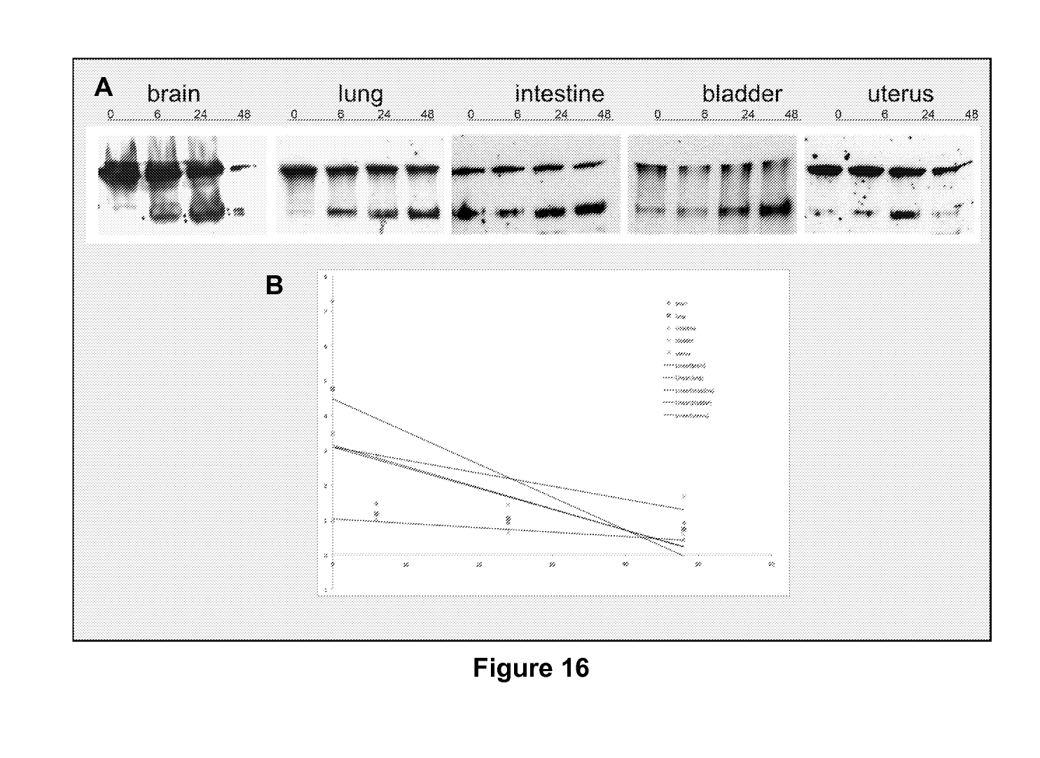

To identify candidate proteins for degradation assessment, a gel-based proteomic analysis was conducted on cultured cells exposed to different cold ischemic times. This gel-based approach allowed for identification of approximate 3,000 protein spots only, and most proteins of the cellular proteome remained undetected. However, this approach allowed for reliable identification of the most highly expressed proteins. Furthermore, it allowed for simultaneous detection of proteins and their breakdown products. To overcome heterogeneity issues of surgical specimens, cultured cells of varied types were used for initial TDI discovery, followed by validation of candidate proteins in human and mouse tissues (FIG. 16).

After selection and excision of candidate protein spots from 2D gels, consensus IDs were identified for 26 proteins by both LC/MS and MALDI/TOF (Table 2). Most of these proteins are highly-expressed and known to participate in degradation-related cellular events, such as membrane repair (#1 AHNAK) (Miracle et al., 2009, PLoS One. 4:e4491; Weiss et al., 2009, Ann Thorac Surg. 88:543-550), membrane/skeleton stabilization (#2 SPTAN1, #4 GSN, #5 VIM, #12 TPM1, #17 TPM3, #20 ACTB, #23 TUBA1B, #27 DCTN2) (Zhang et al., 2009, Apoptosis 14:1289-1298; Hashida et al., 2000, Free Radic Res. 33:147-156; Hwang et al., 1996, Drug Metab Dispos. 24:377-82; Cross et al., 1987, Ann Intern Med. 107:526-545; Allen et al., 1992, Ren Fail. 14:453-460; Krokavec et al., 1987, Vet Med (Praha). 32:145-150; Glantz et al., 2007, Biochemistry 46:502-513; Simonovic et al., 2006, J. Biol. Chem. 281:34333-34340), RNA/protein synthesis (#3 EEF2, #8 EIF3I, #13 B23, #14 NHRNPC, #15 SFRS1, #18 NACA, #25 PDIA6) (Nedrelow et al., 2003, J. Biol. Chem. 278:7735-7741; Huh et al., 2001, Neurosci. Lett. 316:41-44; Brown et al., 1999, J. Biol. Chem. 274:23256-23262; Wang et al., 1998, J. Biol. Chem. 273:22490-22497; Stabach et al., 1997, Biochemistry. 36:57-65; Harris et al., 1988, J. Biol. Chem. 263:15754-61571; Fox et al., 1987, Blood 69:537-545; Harris et al., 1990, Proc. Natl. Acad. Sci. USA. 87:3009-3013; Harris et al., 1989, J. Biol. Chem. 264:17401-17408; Croall et al., 1986, Biochim. Biophys. Acta. 882:287-296), cell death (#6 STRAP, #9 PPP2CA, #10 SET) (Teodori et al., 2002, Cytometry. 49:113-118; Piredda et al., 1997, Cell Death Differ. 4:463-472; Dong et al., 1997, Am J. Pathol. 151:1205-1213; Dietz, Curr. Pharm. Biotechnol. 11:167-174), chromosome segregation (#7 NUP37) (Chung et al., 2008, Int. J. Pharm. 354:16-22), free radical detoxification (#11 PEX19) (Kardinal et al., 1999, Ann. NY Acad. Sci. 886:289-292; Zhang et al., Bioconjug Chem. 22:1410-1415), and cell protection (#16 DDAH1, #19 HSPB1) (Bidwell et al., Adv. Drug Deliv. Rev. 62:1486-96; Sakuma et al., 2012, Eur. J. Pharm. Biopharm. 2012). Additionally identified were key elements in the ubiquitin-proteasome pathway including ubiquitin-like modifier activating enzyme 1 (#21 UBA1) which catalyzes the first step in ubiquitin conjugation to mark cellular proteins for degradation, and two ATPases (#24 PSMC3, #26 PSMC4) on the 26S proteasome which cleaves peptides in an ATP/ubiquitin-dependent process (Shitanda et al., 2012, Anal. Sci. 27:1049-1052; Zhang et al., 2012, Opt. Lett. 35:2143-2145; Mata et al., 2005, Biomed. Microdevices 7:281-293; Fan et al., 2008, Nat. Mater. 7:303-307). Some of these proteins are cell housekeeping proteins suggesting wide applicability as potential TDI's in human tissue specimens.

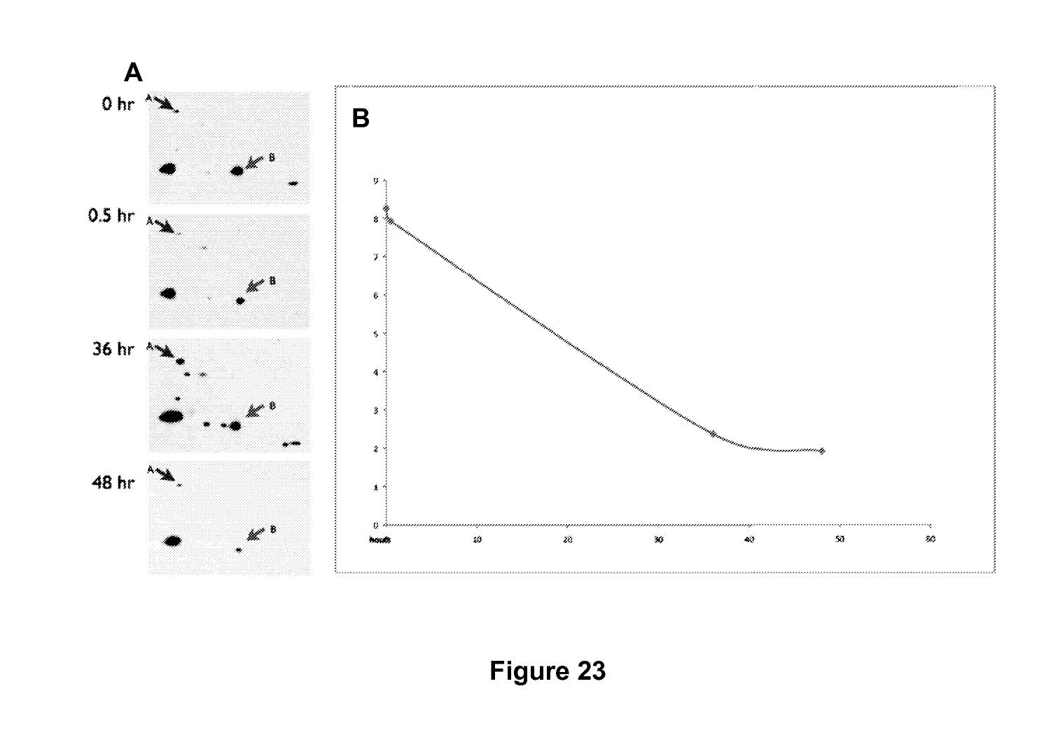

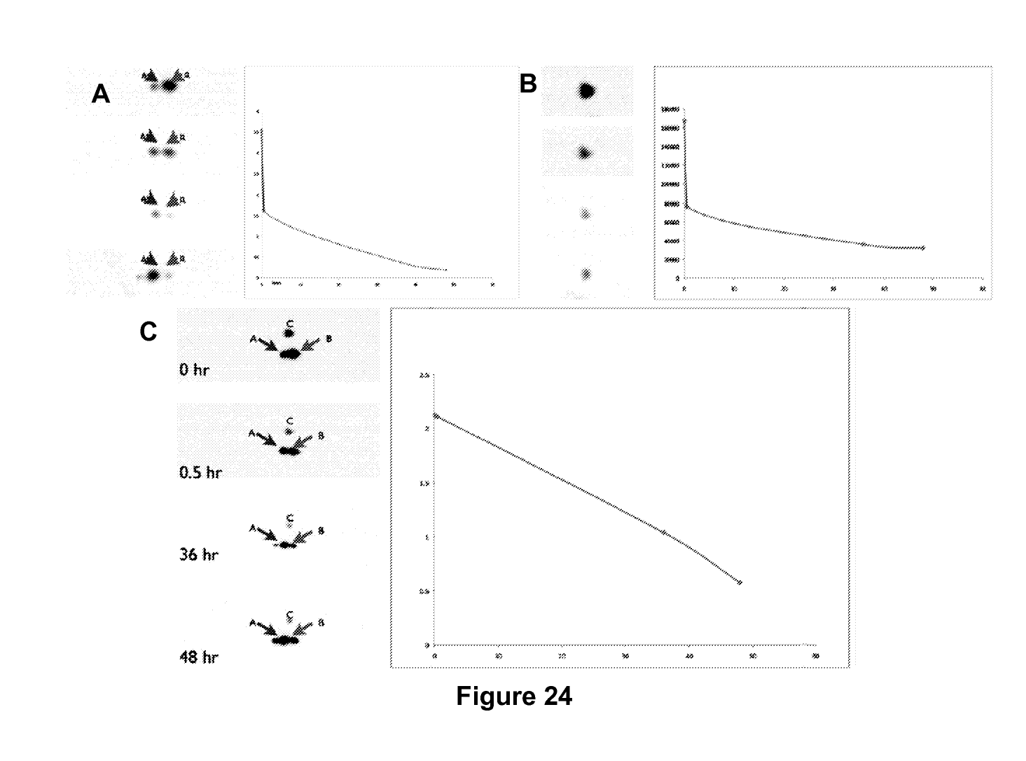

Proteomic findings in cell cultures were validated by Western analysis of an independent cohort of tissue specimens (FIGS. 3-4 and FIGS. 22-23). These experiments confirmed the degradation-associated change of the selected proteins, which is characterized by quantitative loss, accumulation of degradation products, and/or altered isoelectric points. Of particular interest, the quantitative ratio between intact TDIs and their respective breakdown form(s) were used for degradation assessment. Establishment of intensity ratios between intact protein and breakdown forms(s) was found to be a particularly precise and sensitive way for degradation measurement.

Among the identified TDIs, alpha-II spectrin exhibited particularly stable and reproducible kinetics of intact-breakdown conversion within 48 hours of tissue degradation which can be considered as the most representative and relevant time frame for surgical and autopsy tissues (FIG. 5 and FIG. 6). The association between spectrin degradation and the impact of CIT ranged from 0.81 to 0.96 (R² value) in all specimens tested. Alpha II spectrin is a 285 kDa scaffolding protein abundant in most cells. It forms the spectrin heterodimer with any of the five β-spectrins to carryout bewilder functions in cells, such as formation of plasma membrane, maintenance of cell shape (Goldberger et al., 2006, Acc. Chem. Res. 39:239-248; Fan et al., 2005, Phys. Rev. Lett. 95:086607), and participation in signal transduction and molecular trafficking (Hochbaum et al., 2005, Nano. Lett. 5:457-460; Fan et al., 2003, J. Am. Chem. Soc. 125:5254-5255). Alpha-II spectrin breakdown has been extensively studied. Calpains and caspases mediate its proteolysis under a number of pathological conditions in cells, including necrosis, apoptosis, traumatic damage, and cytotoxicity (Takatsuka et al., 2005, Biochem. Biophys. Res. Commun 336:316-323; Zadran et al., 2010, J. Neurosci. 30:1086-1095; Mondello et al., 2010, J. Neurotrauma 27:1203-1213; Lowy et al., 1994, J. Neurochem. 63:886-894; Dutta et al., 2002, Biol. Chem. 383:785-791; Park et al., 2007, Exp. Neurol. 204:49-57; Benz et al., 2008, Int. Immunopharmacol. 8:319-324; Hwang et al., 2008, Neurosci. Lett. 435:251-256; Brophy et al., 2009, J. Neurotrauma 26:471-479). The initial calpain- and caspase-mediated cleavages yield four breakdown products at about 145-150 kDa (Dutta et al., 2002, Biol. Chem. 383:785-791; Wang et al., 1998, J. Biol. Chem. 273:22490-22497; Tamada et al., 2005, Brain Res. 1050:148-155; Nakajima et al., 2011, Invest. Opthamlmol. Vis. Sci. 52:7059-7067; Lee et al., 2011, Parasite Immunol. 33:349-356; Weber et al., 2009, Int. J. Exp. Pathol. 90:387-399; McClung et al., 2009, Am. J. Physiol. Cell Physiol. 296:C363-371). By applying a calpain inhibitor to our degradation model, degradation-induced spectrin breakdown has been successfully blocked and by applying caspase cleavage-specific antibody in Western blotting, the degradation-induced spectrin breakdown products were not detected. Although not wishing to be bound to any particular theory, these data suggest that the degradation-induced spectrin breakdown is mostly calpain-mediated.

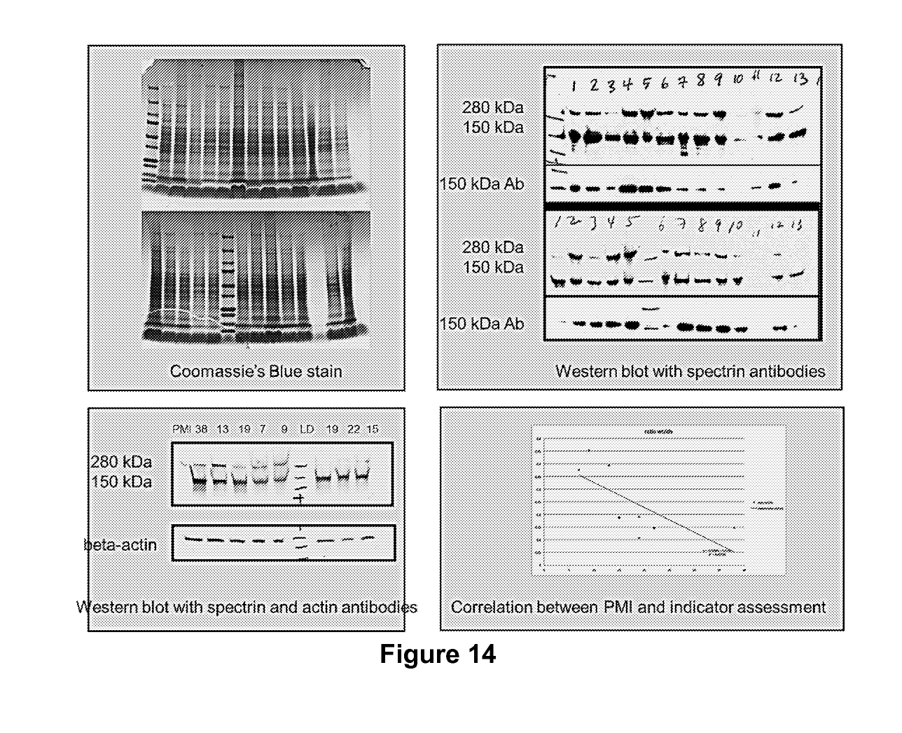

To explore potential further use of TDIs in tissue quality assessment, the correlation between spectrin cleavage ratio and the degradation stage of autopsy tissues was examined in reference to clinically documented PMIs (FIGS. 7A-7B and FIG. 14). In spite of numerous unknown contributing variables, such as circumstances of death, ambient conditions, as well as the accuracy of PMI determination, an association between spectrin cleavage and PMI was detected.

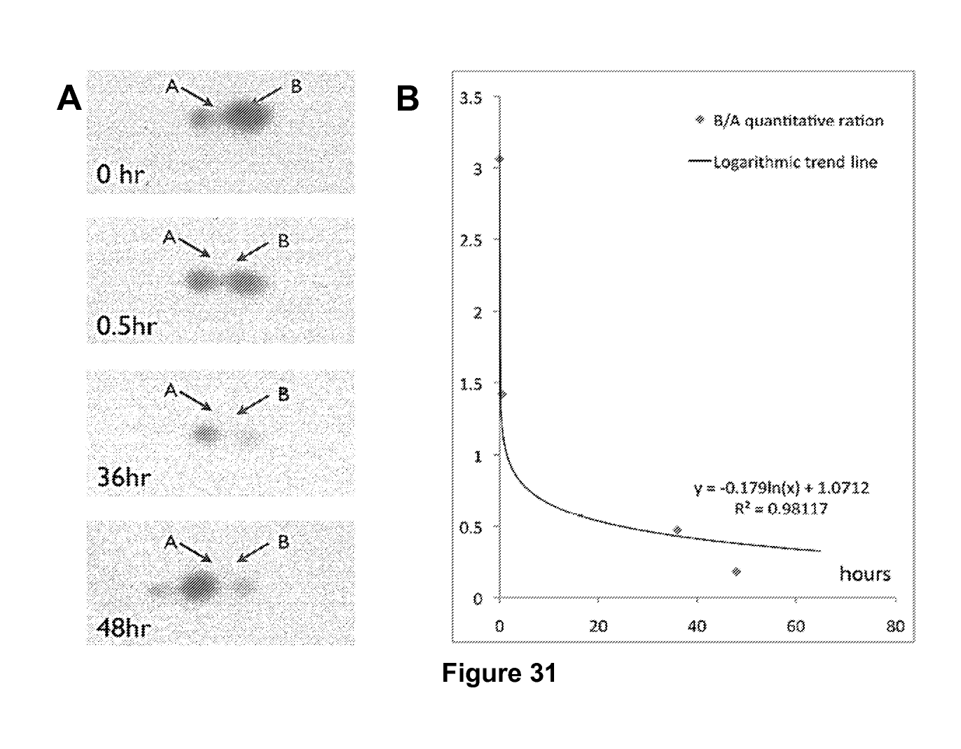

A further attractive and important applications of TDIs are the evaluation of FFPE tissues. Degradation-induced spectrin cleavage was examined in FFPE specimens of varied CIT exposures (FIGS. 7C and 7D). Comparative analysis of the intensity of intact spectrin with the intensity of its breakdown product demonstrated the correlation between spectrin degradation and length of CIT exposed in FFPE specimens (FIG. 31).

### Example 2

### Transformation a Novel Degradation Indicator to a Platform for Specimen Degradation Measurement

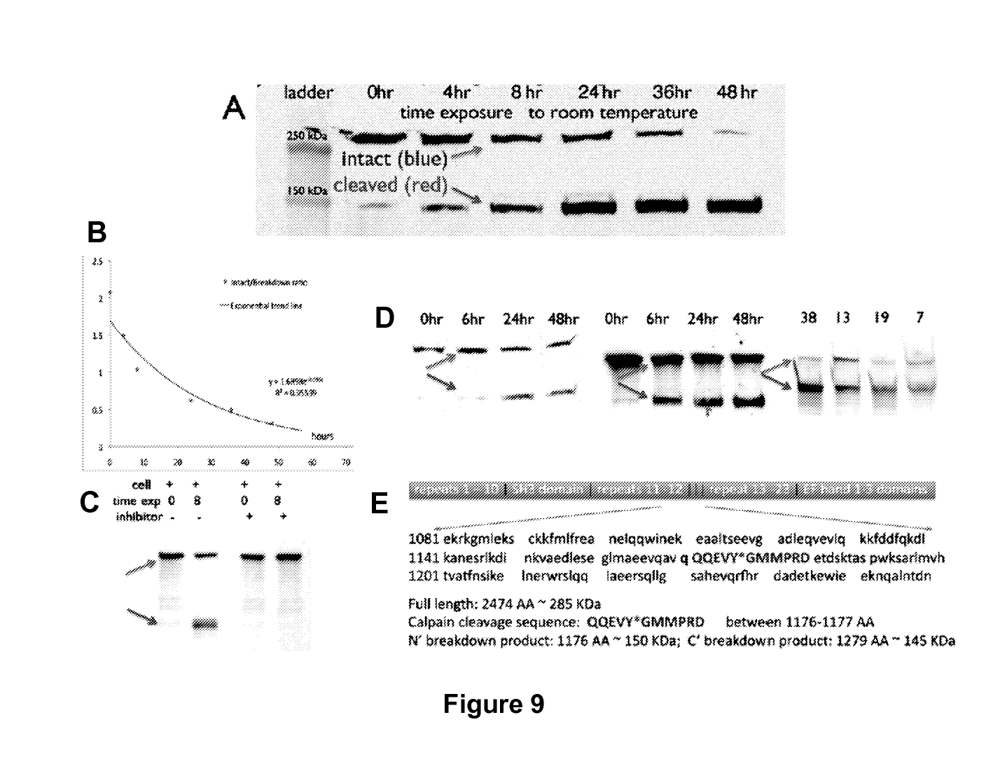

As described herein, comparative proteomic analyses have been performed on multiple human cell lines and specimens of various tissue types that underwent different stage of degradations, using the Stable Isotope Labeling with Amino acids in cell Culture (SILAC) in parallel with the large format 2-D Fluorescence Difference Gel Electrophoresis (2-D DIGE) integrated by LC/MS/MS and MALDI-TOF/MS. Consistent with results from multiple models and multiple proteomic tools, 27 universally presented (housekeeping) Tissue Degradation Indicators have been identified for quantitative degradation measurement; and further, these indicators have been validated in 62 surgical specimens from 12 organ types. Notably, a unique measure for tissue degradation was devised: using a quantitative ratio between the intact molecule and its breakdown form(s) to demonstrate the degradation stage. One degradation indicator, alpha II spectrin, undergoes continuous and dynamic conversion between its intact form and its calpain-dependent cleavage form (proven by calpain-inhibitor assay, FIG. 9B, right) during the process of tissue degradation. The quantitative ratio between these forms is strongly associated (R2 value range between 0.83˜0.95) with the impact of ambient PAVs in observed 62 specimens (FIG. 9).

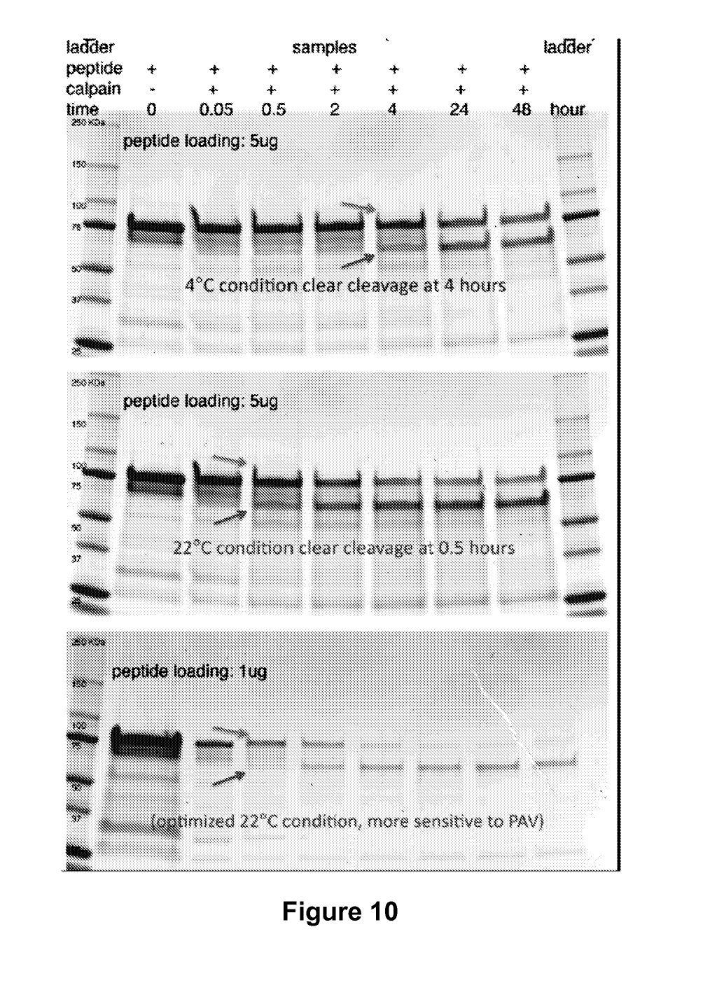

Based on this calpain-mediated spectrin cleavage, as shown in FIG. 10, an “in-tube” enzymatic reaction has been developed which can exclusively imitate the dynamic process of endogenous spectrin cleavage observed in native specimens. Therefore it is used as an ambient PAV monitor, which can provide an important parameter for specimen degradation measurement. More intriguingly, when purified spectrins were introduced (via syringe injection) into the intercellular space of tissue, over a period of degradation time, endogenous calpain vacated the cells of the tissue and conducted in situ cleavage on these extrinsic spectrins following similar kinetics as endogenous spectrins. Although not wishing to be bound to any particular theory, the evacuation of calpain from the cells of the tissue may due to membrane damage.

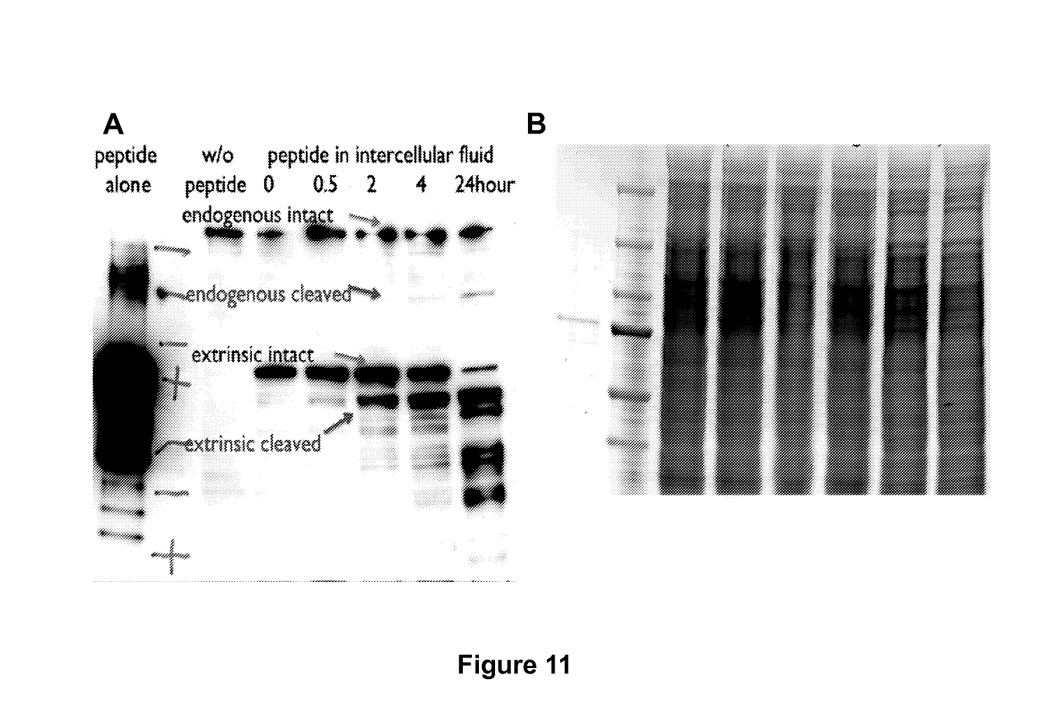

This approach can be used as an in situ PAV monitor, which can provide another important parameter for tissue degradation measurement (FIG. 11, see also Example 4). Taken together, both “in-tube” and “in situ” enzymatic reactions can be imposed immediately after specimen resections (and eternally stay with specimens for receiving identical PAVs). Rather than determining endogenous spectrin cleavage, its analogue—the affiliated enzymatic systems—is examined at anytime for degradation assessment.

To simplify detection towards antibody-free and staining-free procedures (FIG. 8C), FRET-based short spectrin peptides (Vanderklish et al., 2000, Proc. Natl. Acad. Sci. USA 97:2253-2258) can be used as substrates for the enzymatic reaction and the reaction can be quantified with fluorescence readers (Vanderklish et al., 2000, Proc. Natl. Acad. Sci. USA 97:2253-2258; Suzuki et al., 2005, Biochem. Biophys. Res. Commun 330:454-460; Cummings et al., 2002, Proc. Natl. Acad. Sci. USA 99:6603-6606; Kisselev et al., 2006, J. Biol. Chem. 281:8582-8590; Takatsuka et al., 2005, Biochem. Biophys. Res. Commun 336:316-323; Zadran et al., 2010, J. Neurosci. 30:1086-1095). With appropriate controls, the platform monitors and reports FRET-spectrin cleavage in real time and thereby indirectly reveals the degradation stage of respective specimens.

This approach overcomes technical barriers not met by existing methods, and enables cancer specimen users to easily and comprehensively encode the accumulated impact of pre-existing PAVs and consequently revive the original biological profile of specimens. This biological product, all told, acts as an all-in-one timer, thermo-recorder, oxygen-detector, pH meter, and calcium microprobe for example. The Tissue Degradation Indicators can be from human specimens, and capture a real-time snap shot of both ambient environments and tissue microenvironments. The enzymatic reaction follows Michaelis-Menten kinetics and other basic biological regulations and behaves similarly to tissues. Most importantly, the readouts of this system reveal the accumulated impact of all PAVs.

Most other degradation indicators require complicated molecular assays (Western blot, 2D Western blot, 1HC, ELISA, etc.) to reveal the stage of tissue degradation. However, the FRET-spectrin cleavage can be easily visualized or assessed by a single-step fluorescent detector which directly produces quantitative readouts. This technology outperforms any existing assays in terms of the simplicity and cost effectiveness with expected high sensitivity and accuracy.

The spectrin cleavage event is utilized in the development of a global standard for degradation measurement Without such a ‘ruler’ to define the scale, it is not possible to measure the ‘extent’ of degradation between a decomposed specimen and a fresh specimen; neither can its original status be deduced from a decomposed specimen. Using spectrin cleavage as a global standard allows specimen users to obtain a numerical value to define the stage of tissue degradation and thereby conclude the quantitative correlation between the molecule of interest and the tissue degradation stage. When a specimen is studied, two sets of quantitative data can be obtained (from both the quantity of indicator molecule present and the degradation stage of specimen). By referring to their previously concluded correlation, the initial concentration of the indicator can be deduced within the fresh specimen at resection.

### Example 3

### Enzymatic Reporter for Ambient PAV Measurements—the 1^{St} Standard for Tissue Degradation Assessment

Spectrin breakdown is a common outcome from cell damage (Mondello et al., 2010, J. Neurotrauma 27:1203-1213; Lowy et al., 1994, J. Neurochem. 63:886-894; Dutta et al., 2002, Biol. Chem. 383:785-791; Park et al., 2007, Exp. Neurol. 204:49-57; Benz et al., 2008, Int. Immunopharmacol. 8:319-324; Hwang et al., 2008, Neurosci. Lett. 435:251-256; Brophy et al., 2009, J. Neurotrauma 26:471-479; Miracle et al., 2009, PLoS One 4:e4491; Weiss et al., 2009, Ann. Thorac. Surg. 88:543-550; Zhang et al., 2009, Apoptosis 14:1289-1298). Since the calpain-mediated spectrin cleavage has been validated as a real time degradation-dependent indicator, an optimized “in-tube” spectrin cleavage reaction can serve as a biological reporter of time, temperature, atmosphere—the best-known PAVs (Hashida et al., 2000, Free Radic. Res. 33:147-156; Hwang et al., 1996, Drug Metab. Dispos. 24:377-382; Cross et al., 1987, Ann. Intern. Med. 197:526-545; Allen et al., 1992, Ren. Fail. 14:453-460; Krokavec et al., 1987, Vet. Med. (Praha) 32:145-150). With this “in-tube” reaction physically adhered to a specimen at resection, an external PAV monitor accompanies the specimen. The optimized method is the strongest correlation between the quantity of cleavage and the impact of ambient PAVs.

The “in-tube” system using purified spectrin peptides is sensitive to ambient PAV impact (FIG. 10). Similar enzymatic kinetics, such as those observed in native tissues, have been observed despite using different approaches (protein staining vs. Western blotting, FIG. 9). FRET technology is utilized with a short spectrin peptide, thereby permitting spectrin cleavage to be measured by fluorescence emission. A similar strategy with similar sequence has been successfully used for monitoring calpain-mediated spectrin cleavage (Vanderklish et al., 2000, Proc. Natl. Acad. Sci. USA 97:2253-2258).

### FRET-Spectrin Design

Degradation-dependent, calpain-mediated spectrin cleavage occurs between Tryosine-1176 and Glycine-1177 (FIG. 8 and FIG. 9D): This cleavage site is designed to be in the center of the FRET-peptide (SEQ ID NO: 32-QQEVYGMMPRD), flanked by glycine/serine residues, and tagged with 6˜8 His for balancing the PI of the peptide and for potential purification (PI: 7.18, MW: 2867).

### Fluoro-Quencher Selection

To avoid signal overlapping with other laboratory stains, one of the least frequently used fluorescence dyes, HEX, is selected for the FRET analysis. The dye is coupled with 4 quenchers: Dabcyl, BHQ-1, BHQ-2, and Iowa Black, and synthesized at Yale Keck Peptide Synthesize Facility. FRET-spectrin undergo calpain cleavage in tubes in PAV-models and the best fluorescence/quencher pair to reflect the impact of PAVs is selected and synthesized at microgram scale. At the same time, two control peptides are synthesized accordingly: positive control 1: FRET-spectrin without a quencher; positive control 2: similar to 1, but labeled with a complementary fluorophore (for use in Example 4).

### PAV-Degradation Experimental Models

A total of n=9 groups are used. Temperature and time-dependent models include 48 observation points (every 0.5 hour until 24 hours) under three different temperatures (4° C., 22° C., and 37° C.) and a total of n=3 groups. The hypoxia and time-dependent model are examined under three different temperatures with two possible conditions (under ambient atmosphere or “low O₂-high CO₂”). The models are exposure to above time points and temperatures, and a total of n=6 groups are examined.

### Optimization of Enzymatic Condition

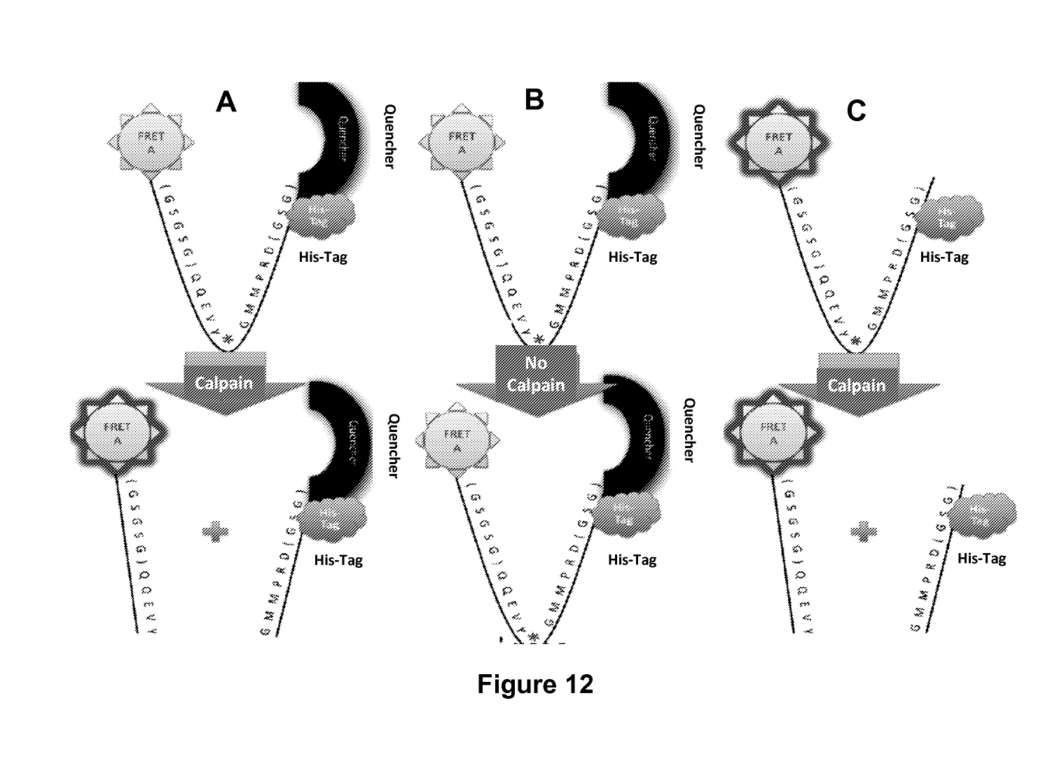

FRET-spectrin and controls (FIG. 12) are incubated with or without calpain (EMD Chemicals Inc, MA) and undergo the previously-mentioned n=9 PAV-groups in 96-wells covered with 76-micron polydimethylsiloxane membrane (PDMS, Specialty Silicone Products, Inc. NY) to allow for air penetration. By titering each component (spectrin, calpain, calcium, sodium, Tris, DTT, and pH) inside the reaction, an optimal kinetic curve is generated which has the strongest association with each PAV impact (yield one regression curve with a R² value for each of the 9 groups) and a method which leads to the best combined R² value for every group.

### Fluorescence Measurement and Data Interpretation

At assigned time points, fluorescence is measured by an automated FLUOstar Optima fluoro-reader (BMG LABTECH, NY) which offers dynamic measurement with integrated O₂/CO₂ and temperature controls. The quantitative ratio of the quantity of experimental spectrin to the quantity of spectrin in the negative control=percentage of cleavage (FIGS. 12B and 12D); ratio of the quantity of experimental spectrin to the quantity of spectrin in the positive control=percentage of intact spectrin (FIG. 12C); ratio of the quantity of spectrin in the positive control to the quantity of spectrin in the negative control=percentage of remaining fluorophore potency; coordination of above parameters yields an algorithm for calculation of an extrinsic “Tissue Degradation Index” (eTDI) which reveals the overall tissue quality loss via ambient PAVs impact. eTDI readouts range from 0% to 100% (quality loss).

### Quality Controls

During system optimization, the enzymatic reactions are examined in parallel using an HP1090 HPLC system with a C-18 reverse-phase column at Yale Keck Facility. To account for any changes in the potency of calpain, an independent “spectrin-free” reaction will be run concurrent with other reactions. At the beginning of observation and at each observation time “fresh” spectrins will be added to initiate enzymatic reaction.

The “in-tube” platform conducts the best assessment for ambient PAVs and results in the first-developed standard for degradation measurement. The ratio of 1 ug spectrin to 5 ng calpain in a reaction may last for 48 hours under 22° C. Alternatively, two reaction types are developed with different methods to provide either short-term or long-term degradation assessment. Cleavage is detected in a 10-minute degradation time under common enzymatic conditions (22° C., 10 ng substrate, ing calpain, 50 uM calcium); with the FRET-spectrin. The kinetic curves of each enzymatic model has a strong association with ambient PAVs (regression curve R² value >0.90), with a batch-to-batch variation <5%.

### Example 4

### An Enzymatic Reporter for In Situ PAV Measurements—the 2n^{d} Standard for Tissue Degradation Assessment

The first parameter for standardization of degradation measurement was described in Example 3. However, this parameter is independent of direct tissue damage and when specimens undergo chemical fixation and histological sectioning, the method will not function. With the understanding that spectrin cleavage is a steady and dynamic indicator for tissue degradation, an additional enzymatic assay for monitoring the in situ spectrin cleavage by manually injecting the FRET-spectrin peptides and its control into the intercellular space of tissue complements the first method. These peptides will undergo endogenous calpain-mediated cleavage, and in doing so, are detected by a fluorescence-detector.

Both in vitro and in vivo spectrin cleavage assays have been demonstrated (Glantz et al., 2007, Biochemistry 46:502-513; Nedrelow et al., 2003, J. Biol. Chem. 278:7735-7741; Huh et al., 2001, Neurosci. Lett. 316:41-44; Brown et al., 1999, J. Biol. Chem. 274:23256-23262; Stabach et al., 1997, Biochemistry 36:57-65; Harris et al., 1988, J. Biol. Chem. 263:15754-15761; Fox et al., 1987, Blood 69:537-545; Harris et al., 1990, Proc. Nat. Acad. Sci. USA 87:3009-3013; Harris et al., 1989, J. Biol. Chem. 264:17401-17408; Croall et al., 1986, Biochim. Biophys. Acta 882:287-296). Preliminary data (FIG. 11) demonstrated that when purified spectrin is introduced into the intercellular space of tissue in a 22° C. time-dependent PAV model, spectrin cleavage is clearly detected within 0.5 hour from intercellular fluid. Trace amount of endogenous spectrin (higher MW) is also detected with a similar cleavage pattern. Although not wishing to be bound by any particular theory, this suggests cytosolic (calpain) leakage due to cell damage (Teodori et al., 2002, Cytometry 49:113-118; Piredda et al., 1997, Cell Death Differ. 4:463-472; Dong et al., 1997, Am. J. Pathol. 151:1205-1213) and likely explains why spectrin cleavage is observed outside the cells. In addition, as in the case of cell-penetrating peptides (CPPs) (Dietz et al., Curr. Pharm. Biotechnol. 11:167-174; Chung et al., 2008, Int. J. Pharm. 354:16-22; Kardinal et al., 1999, Ann. NY Acad. Sci. 886:289-292; Zhang et al., Bioconjug. Chem. 22:1410-1415; Bidwell et al., Adv. Drug Deliv. Rev. 62:1486-1496; Sakuma et al., 2012, Eur. J. Pharma. Biopharma. 81:64-73), this FRET-spectrin peptide is small, has neutral PI and hydrophobicity, wherein these features allow for easy diffusion and membrane penetration.

The materials and methods employed in these experiments are now described.

### Materials and Methods

### Human Cell Lines and Specimens

Cells of 6 common cancer types: MDA-MB-231 (breast), A549 (lung), 293T (kidney), U87 (brain), LNCap (prostate), and DLD-1 (colon) are obtained from ATCC; 5 histologically homogeneous specimens of each above type are collected in the OR at Yale New Haven Hospital following IRB-approved protocols.

### Injection of FRET-Spectrin

FRET-spectrin, mixed with controls (complementary fluorophore and without quencher (FIG. 13), is injected through cell pellets/tissues and undergoes n=9 aforementioned PAV conditions, see Example 3.

### Fluorescence Detection

At observation, a cell smear or a cryostat tissue section is examined by multiphoton fluorescence microscopy under constant light-source energy; 2 types of signal/color are captured: (red) from cleaved FRET-spectrin and (green) independent to cleavage (from control peptides).

### Quantify Fluorescent Signals

Injected as a mixture, the 2 signals spatially overlap in any illuminated location (where the injection is applied), and two numeric values of fluorescence intensity will be generated by embedded software

### Data Interpretation

The quantitative ratio of FRET/control is equal to the percentage of spectrin cleavage and to yield an intrinsic “Tissue Degradation Index” (iTDI) which indicates the overall tissue quality loss caused by in situ PAVs impact. iTDI readouts range from 0% to 100%.

### System Optimization

Using 6 cell lines with 9 PAV conditions, the reaction with a kinetic curve having the strongest association with each PAV impact is first optimized (one regression curve/condition/cell line, with a R² value, n=54) via adjustments of different variables: injection method, quantity, density, the concentration of the mixture, ratio, microscope parameters, until an optimal method is identified which gives the best combined R² value. Additionally, fine adjustments are based on specimen data (5 cases/type, n=30 total) in 9 PAV models: references are obtained for each group one variable at a time until all are evaluated. After taking all data into account, the optimized method yields the best combined R² value for every group.

The two aforementioned parameters (TDIs) are complementary to each other: eTDI is more stable, iTDI reveals more PAV impacts but with more variations. Tissue heterogeneity may affect the readouts between specimens or between locations of a specimen. Therefore, the degradation assessment is conducted in every location used for downstream analysis. Also, the influence of chemical fixatives on a FRET signal is unknown. However, most fixatives will stabilize or cross-link the peptides, thereby stopping the cleavage and resulting in a “degradation-arrested” status. Degradation tests are performed prior to specimen fixation. The system readouts have a strong correlation (R² value >0.90) with each PAVs in all 54 groups. In an optimal protocol the system readouts have a good correlation (R² value >0.80) with each PAV using all 30 surgical specimens.

### Example 5

### System Integration and Assembly of One Prototype Degradation Assessment Platform

Combination use of the two TDIs is required for a more accurate and comprehensive degradation assessment. For commercialization purposes, an ambient PAV reporting system is assembled (Example 3) into a device which can be easily setup in the OR. An algorithm is also generated for a combined TDI to simplify the assessment. The TDI=f (eTDI, iTDI), meaning the TDI determined by the alterations of both eTDI and iTDI, and wherein “f” means “function.” The combined TDI is carried out on clinical specimens.

To keep the enzymatic reaction inactive prior for ease of handling, all reagents are first lyophilized. Inspired by the “bend (for initiation of the reaction)-N-glow (for detection of the fluorescence)”, a prototype model for an ambient PAV reporter is explored by separating lyophilized enzymatic reagents from their solutions until time of use.

The materials and methods employed in this experiment are now described.

### Materials and Methods

### Lyophilized Reagents

All involved elements are generally shipped in lyophilized powder by manufacturers. The reagents are mixed according to optimized concentrations from Examples 3 and 4.

### Prototype Ambient PAV Reporter

FRET-spectrin (FIGS. 13 and 15), and two controls are prefilled into three individual 1 ml-syringes with other lyophilized chemicals. A H₂O ampule and a 3×3 cm-sized 76-micron polydimethylsiloxane membrane are assembled in the same package.

### Reagents for In Situ PAV Reporter

The mixture of lyophilized peptides (FIG. 13) are prefilled into one 10 ml-syringe supplied with a H₂O ampule.

### Specimen Collection

10 cancer specimens are obtained for each of the aforementioned 6 histology types (n=60). Upon resection, H₂O is drawn into ambient PAV syringes, the needle is capped, the reagents are mixed together followed by disposal of the plunger, sand the plunger side is sealed with an air-permeable water-resistant PDMS membrane (to permit air penetration) (Shitana et al., 2012, Anal. Sci. 27:1049-1052; Zhang et al., 2012, Opt. Lett. 35:2143-2145; Mata et al., 2005, Biomed. Microdevices 7:281-293; Fan et al., 2008, Nat. Mater. 7:303-307; Goldberger et al., 2006, Acc. Chem. Res. 39:239-248; Fan et al., 2005, Phys. Rev. Lett. 95:086607; Hochbaum et al. 2005, Nano Lett. 5:457-460; Fan et al., 2003, J. Am. Chem. Soc. 125:5254-5255). The device is attached to the specimen in the tissue bag, and let go assigned PAV models. Concomitantly, reagents for the in situ PAV reporter are dissolved in H₂O and injected throughout the specimen at multiple locations (˜1 cm apart).

### PAV Models

10 specimens of each cancer type are individually exposed to PAV impact of increasing severity (associated with the aggravating impact of miscellaneous PAVs).

eTDI and iTDI Readouts

For the ambient PAV reporter, a small sample of the reaction is drawn from the needle site for fluorescent testing; for in situ reporter, as described herein. FRET-spectrin, mixed with controls (complementary fluorophore with and without quencher), are injected through tissues and at observation, a cryostat tissue section will be examined by multiphoton fluorescence microscopy. Two types of signal/color are captured: (red) from cleaved FRET-spectrin and (green) independent to cleavage, from control peptides. The two signals spatially overlap in any illuminated location where the injection is applied, and two numeric values (fluoro-intensity) will be generated by embedded software. The quantitative ratio of cleaved FRET spectrin to the control is equal to the percentage of spectrin cleavage and thereby yields an intrinsic “Tissue Degradation Index” (iTDI). [[Justin—this was what was stated by the inventor. It is mentioned elsewhere, but I thought I would keep it in here as well]]

### Combined TDI Calculation

The aforementioned tests yield 60 paired values (eTDI and iTDI) dependent upon the severity of PAV impact. These readouts are fed into the TDI=f (eTDI, iTDI) formula to provide final statistics on the algorithm for TDI combination. The TDI algorithm contains a regression factor R²>0.80 to all 60 specimens.

### TDI Clinical Model

In an effort to mimic clinical applications, TDI readouts and clinical diagnostic marker readouts (EGFR in brain cancers and HER2 in breast cancers) are correlated and a demonstrative molecule-specific degradation curve is constructed. The EGFR-/HER²-specific degradation curves contain a value of R²>0.75.

### Example 6

### Subcloning Full Length Human Alpha II Spectrin (7907 Bp) into Insect Expressional Vector

As described herein, this method provides overexpressed large scale full-length human alpha II spectrin with appropriate post-translational modification. The full-length spectrin will be subcloned into an insect expressional vector for a number of reasons. In mammalian cells, the protein is poisonous when overexpressed, and mammalian cells grow too slowly for efficient protein production. In bacteria, while the efficiency of protein production is high, many necessary post-translational modifications are missing, leading to the malfunction of overexpressed proteins. In insect cells, overexpressed proteins are more properly modified (as compared to mammalian or bacterial cells) with an acceptable efficiency of production.

Overexpressed full-length spectrin can be used as control peptides for ELISA assays, for validation of antibodies, as an in vivo or in vitro indicator for the assessment of tissue degradation, and also used to analyze the function of spectrin. To the best of knowledge, there is no full-length spectrin construct or vector available on the market at this time.

The materials and methods employed in this experiment are now described.

Bac-to-Bac® Baculovirus Expression System (Invitrogen, Grand Island, N.Y.) was used. This system provides a rapid and efficient method to generate recombinant baculoviruses which further transport spectrin gene into targeted cells for spectrin production.

The spectrin gene, which was originally cloned in the mammalian expression vector pEYFP-C1 (Clontech, CA), was subcloned into the pFastBac™ HT B donor plasmid. The restriction endonuclease site for Nm 1 was introduced behind the Bam H1 site in pFastBac™ HT B vector with modifications for keeping the insert in frame for spectrin expression. This endonuclease site was used for connection of 5′ of the spectrin gene. SEQ ID NO: 33 describes the restrictive junction of spectrin gene in the pEYFP-C1 vector.

After subcloning, the pFastBac-spectrin construct was transformed into MAX Efficiency® DH10Bac™ competent E. coli cells which contain a baculovirus shuttle vector (bacmid, bMON14272) and a helper plasmid and allows for generation of a recombinant bacmid following transposition of the pFastBac-spectrin. pFastBac-spectrin was incubated with cells on ice for 30 minutes, heat-shock for 45 seconds at 42° C., and then SOC medium was added to the mixture and shaken at 37° C. at 225 rpm for 4 hours. Serially-diluted mixtures were plated on LB agar containing kanamycin, gentamicin, tetracycline, Bluo-gal and IPTG, and incubated at 37° C. for 48 hours.

Blue/white selection was used to identify E. coli colonies with a recombinant bacmid. The colonies were restreaked, and a culture of verified colonies was grown overnight, followed by isolation of the recombinant bacmid DNA as directed by the manufacture's protocol. Bacmid DNA confirmation was PCR-based, using M13 primers and spectrin specific primers.

The Bacmid DNA was used to transfect insect cells using Cellfectin II® reagent. The log-phase Sf9 cells were incubated with DNA-lipid mixture at 27° C. for 4 hours. The medium was replaced by complete growth medium and further incubated for 72 hours. Viral infection could be observed after incubation and media was collected, comprising the P1 viral stock. The P1 stock was amplified by re-infection of Sf9 cells. BaculoTiter assay was used to determine the titer of the virus following standard procedures.

For expression of spectrin, the resulting P2 recombinant baculovirus stock (>107 pfu/mL) was used to infect High Five™ cells following conventional procedures. Cells were lysed resulting in expression of the recombinant spectrin protein which was purified with PureProteome™ nickel magnetic beads (Millipore). The His-tag was removed using AcTEV™ protease (Invitrogen) after purification and the full length human alpha II spectrin was generated.

### Example 7

### Further Evaluation of TDIs

As described herein, validations of selected TDIs using commercially available antibodies have been performed. To simplify detection procedures for potential users, one-dimensional (1D) Western blotting experiments have been utilized.

### Validation of a Large and Complex TDI

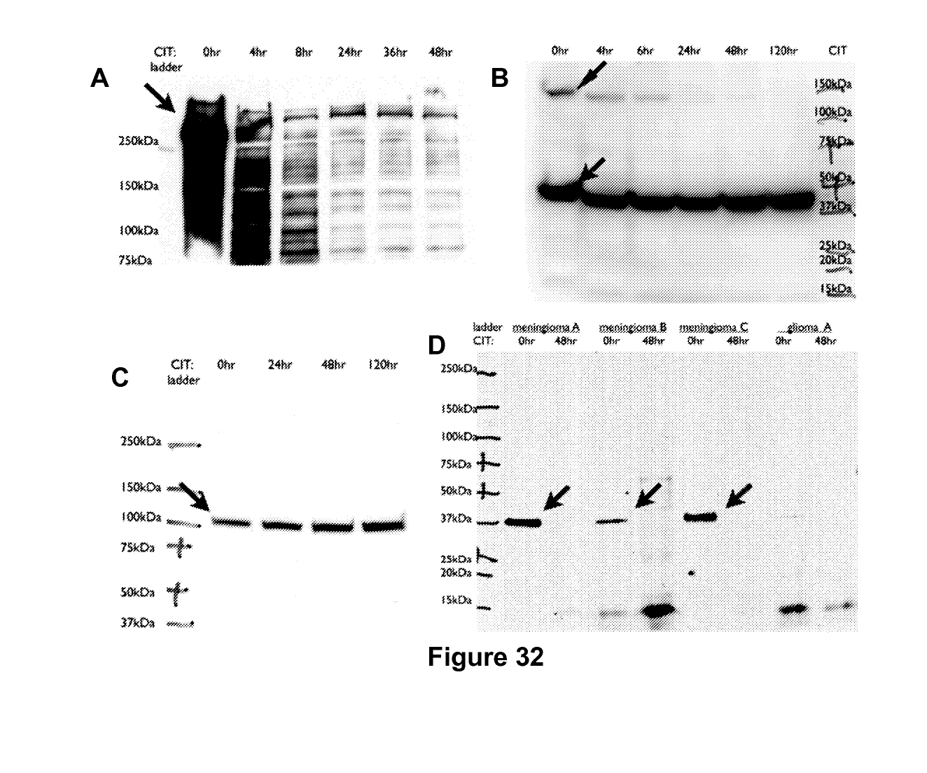

AHNAK protein is a challenging TDI to validate. It is a very large (approximately 600 kD) protein which frequently gets fragmented during protein preparation procedures. Using a commercial anti-AHNAK antibody (FIG. 32A), a single and clear immunosignal was not detected. Instead, remarkable fragmented immunosignals were detected; however, the intensity of all immuno-signals declined during the tissue degradation process. Although some TDIs, such as AHNAK, are not easily validated by conventional Western blot using existing antibodies, they may still serve as good TDIs for other approaches. For example, an AHNAK can be utilized in an ELISA-based detection because the detection will be based on the overall intensity of the immunosignal.

Validation of a Stable Protein that May Serve as Control Marker

Western blot (FIG. 32B) demonstrates that actin-beta maintains a stable quantity at expected MW size (42 kDa, detected by a frequently used monoclonal antibody) during tissue degradation. However, an unexpected 150 kDa band appeared at an early stage of degradation and faded away at a later stage. Although not wishing to be bound to any particular theory, this 150 kDa band may be an actin-participating protein complex that cannot be dissociated by routine cell lysis buffer. Therefore, actin-beta may serve as valuable internal controls for other TDI-based tissue degradation detection assays.

Validation of a Rapidly (within Hours) Degrading TDI

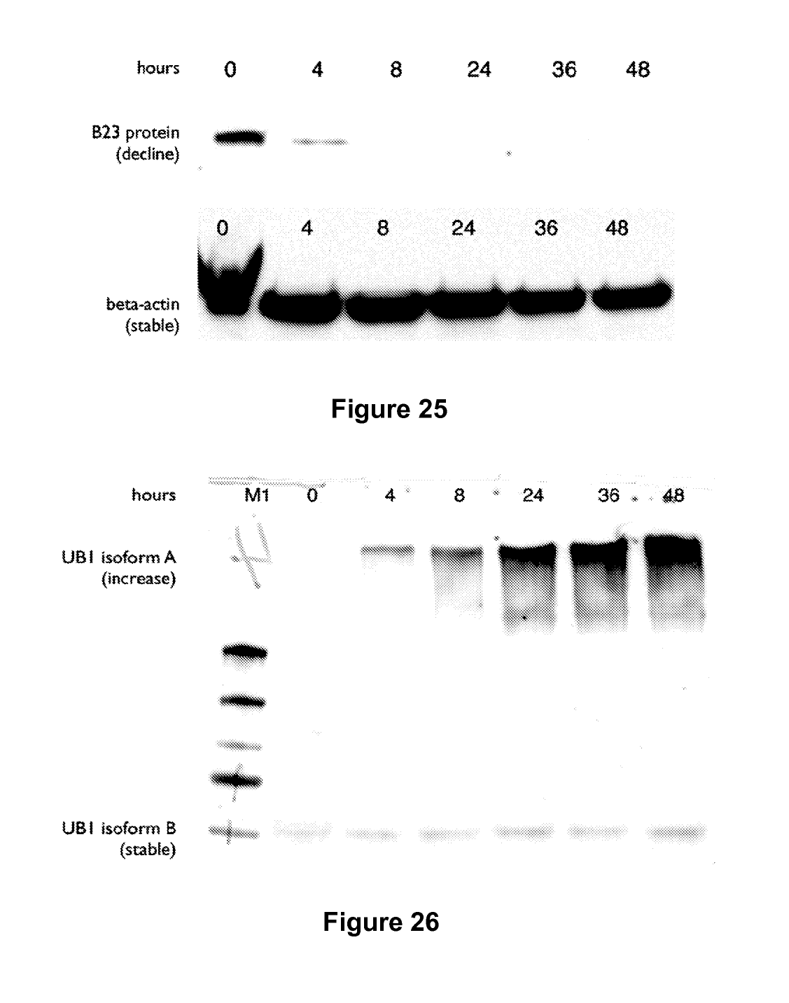

B23 was found to be a rapidly degrading TDI (FIG. 25). FIG. 32D shows the quantitative change of B23 protein at 33 kDa over a 48 h degradation period in four brain tumor specimens. Within 48 h, B23 expression is undetectable. Using antibody-based immunoassay, not every TDI can preserve the dynamic degradation pattern over long degradation period. Therefore, the identified TDIs show variable degradation profiles and may be used individually to assess specific degradation stages of interest.

### Validation of a TDI Whose Expression Increases Over Time

UBEa1 protein was found to accumulate during the tissue degradation process (FIG. 26 and FIG. 32C).

### Example 8

### Exploration of the Degradation Mechanism

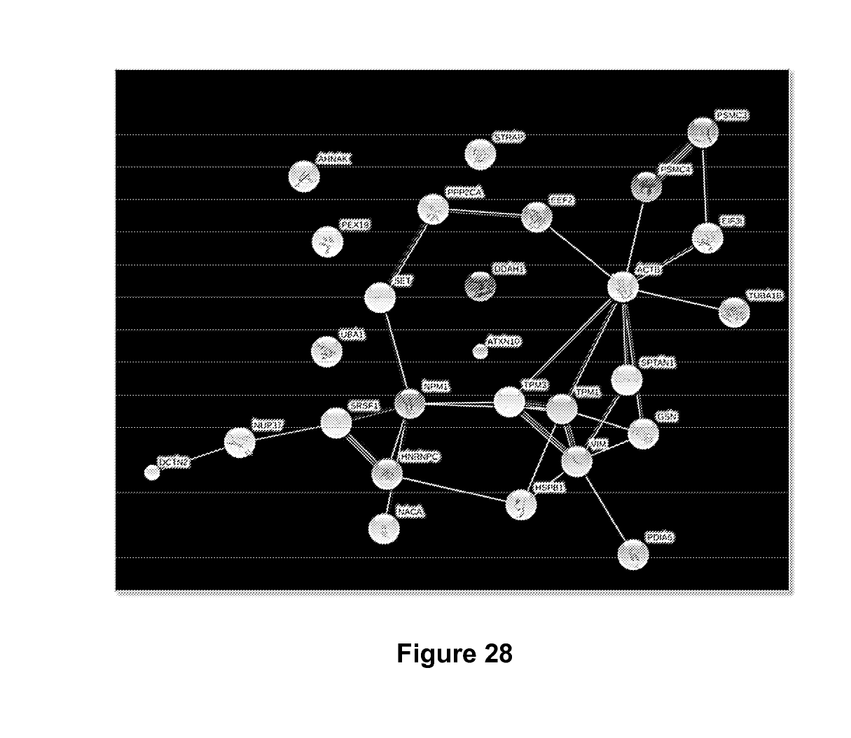

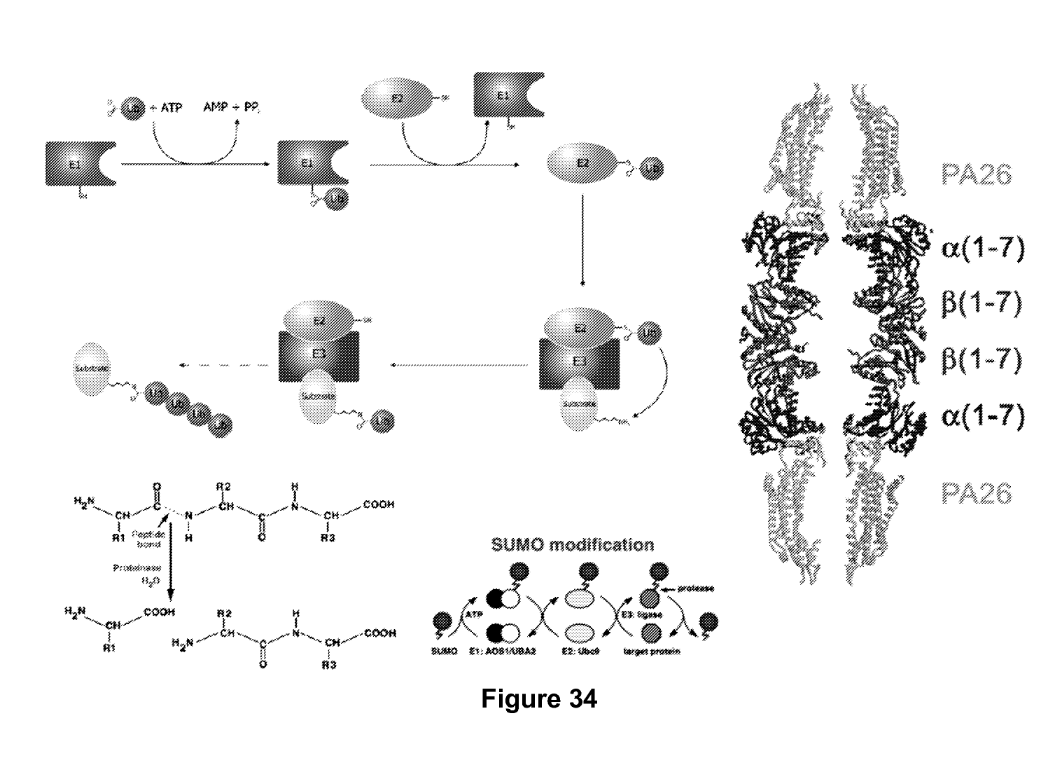

For better understanding the hidden mechanism behind TDIs (FIG. 34), the overall crosstalks among TDIs was investigated. FIG. 28 summarizes the biological relationship between the identified TDIs. It was found that several key elements among the discovered TDIs participate in critical steps of proteolytic degradation, including the ubiquitin-proteasome pathway.

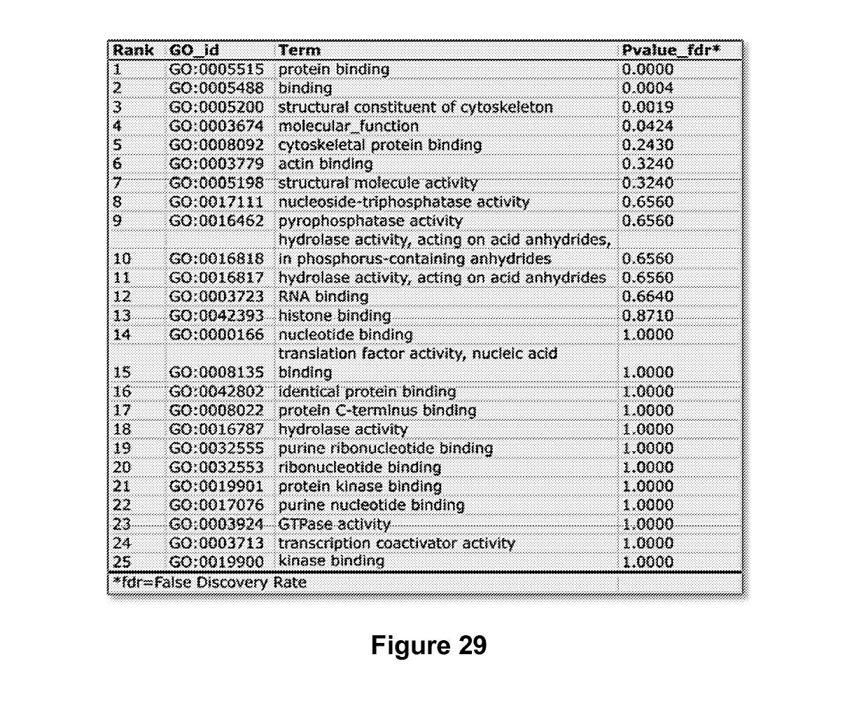

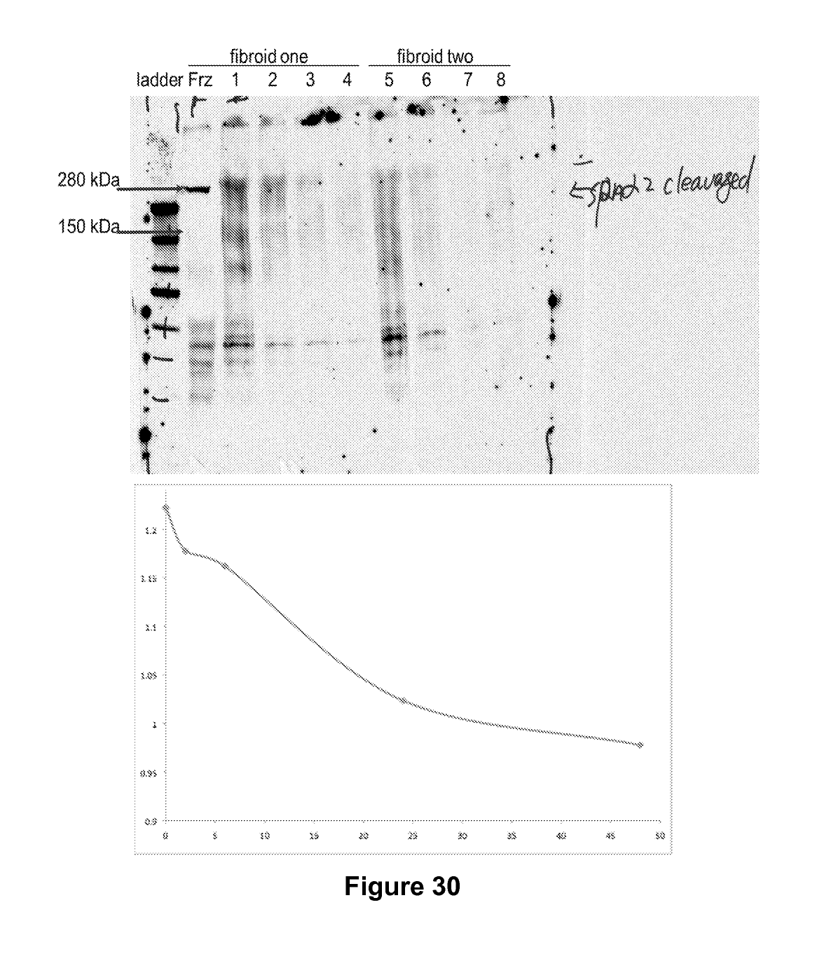

FIG. 29 shows the functional cluster of the identified TDIs using a statistical analysis tool, indicating most TDIs are structural elements for cytoskeleton and conduct massive protein-protein interactions.

### Example 9

### Spectrin Cleavage as a Commercializable Measurement of Tissue Decay

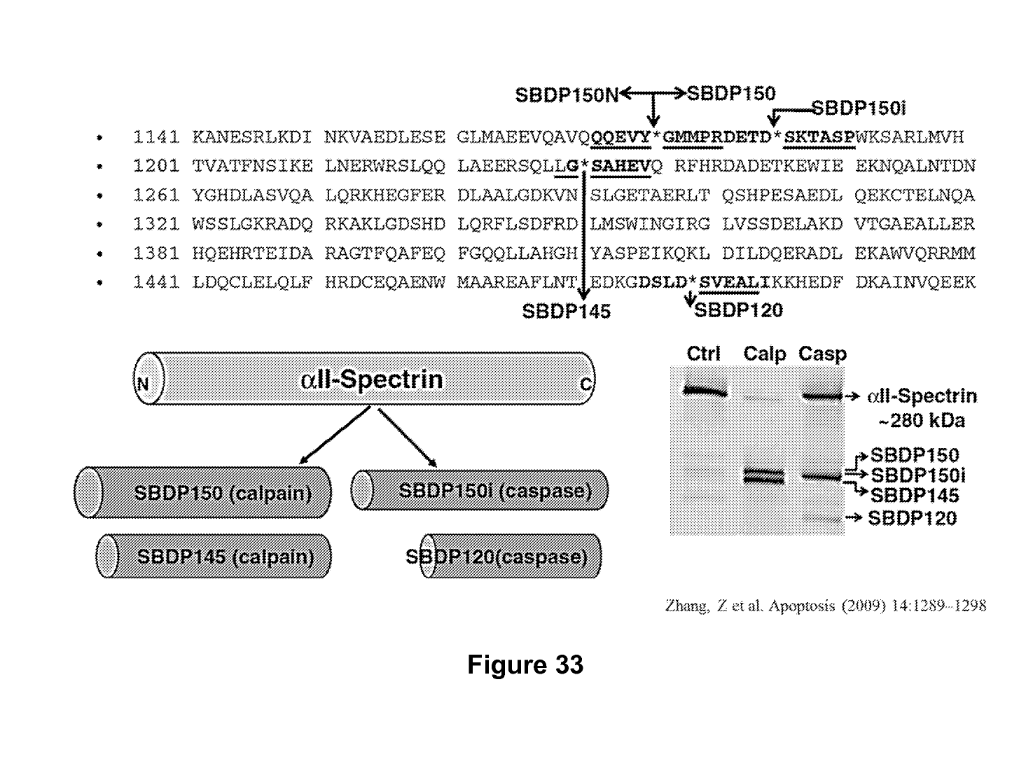

While many of the identified TDIs are promising candidates to evaluate tissue integrity at variable points of decay, spectrin is a particularly attractive TDI because a dynamic conversion exists between intact spectrin and its breakdown form(s) during tissue degradation. This degradation is detectable with antibody-based immunoassays, allowing for sensitive assessment of tissue degradation with little systemic error. Furthermore, degradation-dependent spectrin cleavage is known to be calpain-mediated and this cleavage process has been very well documented in spectrin-related basic research (FIG. 33).

To obtain intact form-specific and cleavage-specific antibodies, multiple epitopes (SEQ ID NO 4 to and through SEQ ID NO 31) have been designed for generation of spectrin antibodies, including N′-specific, C′-specific, cleavage-specific, cleavage site-phospho-specific and full length-specific antibodies.

Purified spectrin peptides can be developed to serve as standards for an ELISA kit, including cloning the full-length spectrin sequence into insect-expressional vectors, cloning the fragmented spectrin sequence (containing calpain cleavage site) into expressional vectors.

Also, an in vitro calpain-spectrin cleavage system was developed which can imitate the endogenous spectrin cleavage process in in vivo tissue degradation. (FIG. 51).

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A method of assessing the amount of degradation in a biological sample, the method comprising:
measuring the level of a biological sample in the biological sample; and
comparing the level of the biological sample to a reference curve, wherein the reference curve correlates the level of the biological sample with the amount of degradation in the biological sample.

2. The method of claim 1, wherein the biological sample is a protein.

3. The method of claim 2, wherein the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2).

4. The method of claim 2, wherein the protein is spectrin.

5. The method of claim 1, wherein the measuring of the biological sample comprises an immunoassay.

6. The method of claim 5, wherein the immunoassay is an ELISA.

7. The method of claim 6, wherein the ELISA comprises antibodies which are directed toward spectrin, any spectrin isoforms, or a calpain-mediated cleavage product of spectrin.

8. The method of claim 6, wherein the antibodies of the ELISA are directed toward epitopes selected from the group consisting of SEQ ID NO: 4 to SEQ ID NO: 32.

9. The method of claim 1, wherein the measuring of the biological sample comprises FRET.

10. The method of claim 9, wherein FRET comprises a peptide selected from the group consisting of a full-length spectrin peptide, a peptide of a fragment of spectrin.

11. The method of claim 10, wherein the peptide of a fragment of spectrin contains the sequence encoding the site of calpain cleavage (SEQ ID NO: 32).

12. The method of claim 9, wherein FRET comprises a fluorescent dye selected from the group consisting of fluorescein, rhodamine, 4-nitrobenzo-2-oxa-1,3-diazole (NBD), cascade blue, 4,4-difluoro-5,7-diphenyl-4-bora-3a,4a-diaza-s-indacene-3-propionic acid, 4,4-difluoro-5,p-methoxyphenyl-4-bora-3a,4a-diaza-s-indacene-3-propionic acid, 4,4-difluoro-5-styryl-4-bora-3a,4a-diaza-s-indacene-propionic acid, 6-carboxy-2′,4,4′,5′,7,7′-hexachlorofluorescein (HEX), 6-carboxy-X-rhodamine, N,N,N′,N′-tetramethyl-6-carboxyrhodamine, iodoacetyl-directed probes such as 5-((((2-iodoacetyl)amino)ethyl)amino)naphthalene-1-sulfonic acid (IAEDANS, used interchangeably with AEDANS), 5-carboxyfluorescein; 6-carboxyfluorescein; 6-(fluorescein-5-carboxamide)hexanoic acid; fluorescein isothiocyanate (FITC), tetramethylrhodamine isothiocyanate (TRITC), Texas Red (TR), eosin, a phycobiliprotein, cyanine dye, coumarin, R-phycoerythrin, allophycoerythrin (APC), a R-phycoerythrin (R-PE) conjugate, an Alexa Fluor dye, a quantum dot dye, maleimide-directed probes such as 4-dimethylaminoazobenzne-4′-maleimide (DABma1) and fluorescein-5-maleimide (Fmal), or a combination thereof.

13. The method of claim 12, wherein the fluorescent dye is HEX.

14. The method of claim 9, wherein FRET comprises a dark quencher The method of claim 9, wherein FRET comprises a dark quencher selected from the group consisting of 4-(dimethylamino)azobenzene (Dabcyl), QSY 35, BHQ-0, Eclipse, BHQ-1, QSY 7, QSY 9, BHQ-2, ElleQuencher, Iowa Black, QSY 21, BHQ-3, or a combination thereof.

15. The method of claim 14, wherein the dark quencher is selected from the group consisting of Dabcyl, BHQ-1, BHQ-2, and Iowa Black.

16. The method of claim 9, wherein FRET comprises a dark quencher/fluorescent dye pair.

17. The method of claim 16, wherein the dark quencher/fluorescent dye pair is selected from the group consisting of HEX/Dabcyl, HEX/BHQ-1, HEX/BHQ-2, and HEX/Iowa Black.

18. The method of claim 9, wherein FRET comprises two fluorescent dyes which emit different colors of light.

19. A kit for assessing the amount of degradation in a biological sample, comprising an assay for measuring the level of a biological sample in the biological sample and comparing the level of the biological sample to a reference curve, wherein the reference curve correlates the level of the biological sample with the amount of degradation in the biological sample.

20. The kit of claim 19, wherein the biological sample is a protein.

21. The kit of claim 20, wherein the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2).

22. The kit of claim 21, wherein the protein is spectrin.

23. The kit of claim 19, wherein the assay is an ELISA.

24. The kit of claim 23, wherein the ELISA comprises antibodies which are directed toward spectrin, any spectrin isoforms, or a calpain-mediated cleavage product of spectrin.

25. The kit of claim 24, wherein the antibodies of the ELISA are directed toward epitopes selected from the group consisting of SEQ ID NO: 4 to SEQ ID NO: 32.

26. A kit comprising a pre-analytical variable monitor for assessing the amount of degradation in a biological sample, comprising an assay for measuring the level of a biological sample in the biological sample and comparing the level of the biological sample to a reference curve, wherein the reference curve correlates the level of the biological sample with the amount of degradation in the biological sample.

27. The kit of claim 26, wherein the pre-analytical variable comprises assessing pre-analytical variables extrinsic to the biological sample.

28. The kit of claim 26, wherein the pre-analytical variable comprises assessing pre-analytical variables intrinsic to the biological sample.

29. The kit of claim 26, wherein the biological sample is a protein.

30. The kit of claim 29, wherein the protein is selected from the group consisting of AHNAK nucleoprotein, human alpha-II spectrin (SPTAN1), eukaryotic translation elongation factor 2 (EEEF2), gelsolin (GSN), vimentin (VIM), serine/threonine kinase receptor associated protein (STRAP), nucleoporin (NUP37, 37 kDa), eukaryotic translation initiation factor 3 (EIF3I), subunit I, protein phosphatase 2, catalytic subunit, alpha isozyme, SET, SET nuclear oncogene, PEX19, peroxisomal biogenesis factor 19 (PPP2CA-001), tropomyosin 1 (alpha) (TMP1), nucleophosmin (nucleolar phosphoprotein B23, numatrin) (NPM1P21), heterogeneous nuclear ribonucleoprotein C(C1/C2) (HNRNPC), pre-mRNA-splicing factor SF2, P33 subunit (SFRS1), dimethylarginine dimethylaminohydrolase 1 (DDAH1), tropomyosin 3 (TPM3), nascent polypeptide-associated complex subunit alpha (NAC-alpha) (NACA), heat shock 27 kDa protein 1 (HSPB1), actin-beta (ACTB), ubiquitin-like modifier activating enzyme 1 (UBA1), ataxin 10 (ATXN10), tubulin (TUBA1B), alpha 1b, proteasome (prosome, macropain) 26S subunit ATPase 3 (PSMC3), protein disulfide isomerase family A member 6 (PDIA6), proteasome (prosome, macropain) 26S subunit ATPase 4 (PSMC4), and dynactin 2 (DCTN2).

31. The kit of claim 30, wherein the protein is spectrin.

32. The kit of claim 26, wherein the measuring of the biological sample comprises FRET.

33. The kit of claim 32, wherein FRET comprises a peptide selected from the group consisting of a full-length spectrin peptide, a peptide of a fragment of spectrin.

34. The kit of claim 33, wherein the peptide of a fragment of spectrin contains the sequence encoding the site of calpain cleavage (SEQ ID NO: 32).

35. The kit of claim 32, wherein FRET comprises a fluorescent dye selected from the group consisting of fluorescein, rhodamine, 4-nitrobenzo-2-oxa-1,3-diazole (NBD), cascade blue, 4,4-difluoro-5,7-diphenyl-4-bora-3a,4a-diaza-s-indacene-3-propionic acid, 4,4-difluoro-5,p-methoxyphenyl-4-bora-3a,4a-diaza-s-indacene-3-propionic acid, 4,4-difluoro-5-styryl-4-bora-3a,4a-diaza-s-indacene-propionic acid, 6-carboxy-2′,4,4′,5′,7,7′-hexachlorofluorescein (HEX), 6-carboxy-X-rhodamine, N,N,N′,N′-tetramethyl-6-carboxyrhodamine, iodoacetyl-directed probes such as 5-((((2-iodoacetyl)amino)ethyl)amino)naphthalene-1-sulfonic acid (IAEDANS, used interchangeably with AEDANS),5-carboxyfluorescein; 6-carboxyfluorescein; 6-(fluorescein-5-carboxamide)hexanoic acid; fluorescein isothiocyanate (FITC), tetramethylrhodamine isothiocyanate (TRITC), Texas Red (TR), eosin, a phycobiliprotein, cyanine dye, coumarin, R-phycoerythrin, allophycoerythrin (APC), a R-phycoerythrin (R-PE) conjugate, an Alexa Fluor dye, a quantum dot dye, maleimide-directed probes such as 4-dimethylaminoazobenzne-4′-maleimide (DABma1) and fluorescein-5-maleimide (Fmal), or a combination thereof.

36. The kit of claim 35, wherein the fluorescent dye is HEX.

37. The kit of claim 32, wherein FRET comprises a dark quencher The method of claim 9, wherein FRET comprises a dark quencher selected from the group consisting of 4-(dimethylamino)azobenzene (Dabcyl), QSY 35, BHQ-0, Eclipse, BHQ-1, QSY 7, QSY 9, BHQ-2, ElleQuencher, Iowa Black, QSY 21, BHQ-3, or a combination thereof.

38. The kit of claim 37, wherein the dark quencher is selected from the group consisting of Dabcyl, BHQ-1, BHQ-2, and Iowa Black.

39. The kit of claim 32, wherein FRET comprises a dark quencher/fluorescent dye pair.

40. The kit of claim 39, wherein the dark quencher/fluorescent dye pair is selected from the group consisting of HEX/Dabcyl, HEX/BHQ-1, HEX/BHQ-2, and HEX/Iowa Black.

41. The kit of claim 32, wherein FRET comprises two fluorescent dyes which emit different colors of light.

42. A kit for assessing the amount of degradation in a biological sample, comprising an indicator strip for measuring the level of a biological sample in the biological sample and comparing the level of the biological sample to a reference curve, wherein the reference curve correlates the level of the biological sample with the amount of degradation in the biological sample.


SEQUENCE LISTING (33 sequences)

SEQ ID NO: 1 - protein, 2477 aa, Homo sapiens
MDPSGVKVLETAEDIQERRQQVLDRYHRFKELSTLRRQKLEDSYRFQFFQRDAEELEKWIQEKLQIASDENYKDPTNLQGKLQKHQAFEAEVQANSGAIVKLDETGNLMISEGHFASETIRTRLMELHRQWELLLEKMREKGIKLLQAQKLVQYLRECEDVMDWINDKEAIVTSEELGQDLEHVEVLQKKFEEFQTDMAAHEERVNEVNQFAAKLIQEQHPEEELIKTKQDEVNAAWQRLKGLALQRQGKLFGAAEVQRFNRDVDETISWIKEKEQLMASDDFGRDLASVQALLRKHEGLERDLAALEDKVKALCAEADRLQQSHPLSATQIQVKREELITNWEQIRTLAAERHARLNDSYRLQRFLADFRDLTSWVTEMKALINADELASDVAGAEALLDRHQEHKGEIDAHEDSFKSADESGQALLAAGHYASDEVREKLTVLSEERAALLELWELRRQQYEQCMDLQLFYRDTEQVDNWMSKQEAFLLNEDLGDSLDSVEALLKKHEDFEKSLSAQEEKITALDEFATKLIQNNHYAMEDVATRRDALLSRRNALHERAMRRRAQLADSFHLQQFFRDSDELKSWVNEKMKTATDEAYKDPSNLQGKVQKHQAFEAELSANQSRIDALEKAGQKLIDVNHYAKDEVAARMNEVISLWKKLLEATELKGIKLREANQQQQFNRNVEDIELWLYEVEGHLASDDYGKDLTNVQNLQKKHALLEADVAAHQDRIDGITIQARQFQDAGHFDAENIKKKQEALVARYEALKEPMVARKQKLADSLRLQQLFRDVEDEETWIREKEPIAASTNRGKDLIGVQNLLKKHQALQAEIAGHEPRIKAVTQKGNAMVEEGHFAAEDVKAKLHELNQKWEALKAKASQRRQDLEDSLQAQQYFADANEAESWMREKEPIVGSTDYGKDEDSAEALLKKHEALMSDLSAYGSSIQALREQAQSCRQQVAPTDDETGKELVLALYDYQEKSPREVTMKKGDILTLLNSTNKDWWKVEVNDRQGFVPAAYVKKLDPAQSASRENLLEEQGSIALRQEQIDNQTRITKEAGSVSLRMKQVEELYHSLLELGEKRKGMLEKSCKKFMLFREANELQQWINEKEAALTSEEVGADLEQVEVLQKKFDDFQKDLKANESRLKDINKVAEDLESEGLMAEEVQAVQQQEVYGMMPRDETDSKTASPWKSARLMVHTVATFNSIKELNERWRSLQQLAEERSQLLGSAHEVQRFHRDADETKEWIEEKNQALNTDNYGHDLASVQALQRKHEGFERDLAALGDKVNSLGETAERLIQSHPESAEDLQEKCTELNQAWSSLGKRADQRKAKLGDSHDLQRFLSDFRDLMSWINGIRGLVSSDELAKDVTGAEALLERHQEHRTEIDARAGTFQAFEQFGQQLLAHGHYASPEIKQKLDILDQERADLEKAWVQRRMMLDQCLELQLFHRDCEQAENWMAAREAFLNTEDKGDSLDSVEALIKKHEDFDKAINVQEEKIAALQAFADQLIAAGHYAKGDISSRRNEVLDRWRRLKAQMIEKRSKLGESQTLQQFSRDVDEIEAWISEKLQTASDESYKDPTNIQLSKLLSKHQKHQAFEAELHANADRIRGVIDMGNSLIERGACAGSEDAVKARLAALADQWQFLVQKSAEKSQKLKEANKQQNFNTGIKDFDFWLSEVEALLASEDYGKDLASVNNLLKKHQLLEADISAHEDRLKDLNSQADSLMTSSAFDTSQVKDKRDTINGRFQKIKSMAASRRAKLNESHRLHQFFRDMDDEESWIKEKKLLVGSEDYGRDLTGVQNLRKKHKRLEAELAAHEPAIQGVLDTGKKLSDDNTIGKEEIQQRLAQFVEHWKELKQLAAARGQRLEESLEYQQFVANVEEEEAWINEKMTLVASEDYGDTLAAIQGLLKKHEAFETDFTVHKDRVNDVCTNGQDLIKKNNHHEENISSKMKGLNGKVSDLEKAAAQRKAKLDENSAFLQFNWKADVVESWIGEKENSLKTDDYGRDLSSVQTLLTKQETFDAGLQAFQQEGIANITALKDQLLAAKHVQSKAIEARHASLMKRWSQLLANSAARKKKLLEAQSHFRKVEDLFLTFAKKASAFNSWFENAEEDLTDPVRCNSLEEIKALREAHDAFRSSLSSAQADFNQLAELDRQIKSFRVASNPYTWFTMEALEETWRNLQKIIKERELELQKEQRRQEENDKLRQEFAQHANAFHQWIQETRTYLLDGSCMVEESGTLESQLEATKRKHQEIRAMRSQLKKIEDLGAAMEEALILDNKYTEHSTVGLAQQWDQLDQLGMRMQHNLEQQIQARNTTGVTEEALKEFSMMFKHFDKDKSGRLNHQEFKSCLRSLGYDLPMVEEGEPDPEFEAILDTVDPNRDGHVSLQEYMAFMISRETENVKSSEEIESAFRALSSEGKPYVTKEELYQNLTREQADYCVSHMKPYVDGKGRELPTAFDYVEFTRSLFVN

SEQ ID NO: 2 - protein, 2452 aa, Homo sapiens
MDPSGVKVLETAEDIQERRQQVLDRYHRFKELSTLRRQKLEDSYRFQFFQRDAEELEKWIQEKLQIASDENYKDPTNLQGKLQKHQAFEAEVQANSGAIVKLDETGNLMISEGHFASETIRTRLMELHRQWELLLEKMREKGIKLLQAQKLVQYLRECEDVMDWINDKEAIVTSEELGQDLEHVEVLQKKFEEFQTDMAAHEERVNEVNQFAAKLIQEQHPEEELIKTKQDEVNAAWQRLKGLALQRQGKLFGAAEVQRFNRDVDETISWIKEKEQLMASDDFGRDLASVQALLRKHEGLERDLAALEDKVKALCAEADRLQQSHPLSATQIQVKREELITNWEQIRTLAAERHARLNDSYRLQRFLADFRDLTSWVTEMKALINADELASDVAGAEALLDRHQEHKGEIDAHEDSFKSADESGQALLAAGHYASDEVREKLTVLSEERAALLELWELRRQQYEQCMDLQLFYRDTEQVDNWMSKQEAFLLNEDLGDSLDSVEALLKKHEDFEKSLSAQEEKITALDEFATKLIQNNHYAMEDVATRRDALLSRRNALHERAMRRRAQLADSFHLQQFFRDSDELKSWVNEKMKTATDEAYKDPSNLQGKVQKHQAFEAELSANQSRIDALEKAGQKLIDVNHYAKDEVAARMNEVISLWKKLLEATELKGIKLREANQQQQFNRNVEDIELWLYEVEGHLASDDYGKDLTNVQNLQKKHALLEADVAAHQDRIDGITIQARQFQDAGHFDAENIKKKQEALVARYEALKEPMVARKQKLADSLRLQQLFRDVEDEETWIREKEPIAASTNRGKDLIGVQNLLKKHQALQAEIAGHEPRIKAVTQKGNAMVEEGHFAAEDVKAKLHELNQKWEALKAKASQRRQDLEDSLQAQQYFADANEAESWMREKEPIVGSTDYGKDEDSAEALLKKHEALMSDLSAYGSSIQALREQAQSCRQQVAPTDDETGKELVLALYDYQEKSPREVTMKKGDILTLLNSTNKDWWKVEVNDRQGFVPAAYVKKLDPAQSASRENLLEEQGSIALRQEQIDNQYHSLLELGEKRKGMLEKSCKKFMLFREANELQQWINEKEAALTSEEVGADLEQVEVLQKKFDDFQKDLKANESRLKDINKVAEDLESEGLMAEEVQAVQQQEVYGMMPRDETDSKTASPWKSARLMVHTVATFNSIKELNERWRSLQQLAEERSQLLGSAHEVQRFHRDADETKEWIEEKNQALNTDNYGHDLASVQALQRKHEGFERDLAALGDKVNSLGETAERLIQSHPESAEDLQEKCTELNQAWSSLGKRADQRKAKLGDSHDLQRFLSDFRDLMSWINGIRGLVSSDELAKDVTGAEALLERHQEHRTEIDARAGTFQAFEQFGQQLLAHGHYASPEIKQKLDILDQERADLEKAWVQRRMMLDQCLELQLFHRDCEQAENWMAAREAFLNTEDKGDSLDSVEALIKKHEDFDKAINVQEEKIAALQAFADQLIAAGHYAKGDISSRRNEVLDRWRRLKAQMIEKRSKLGESQTLQQFSRDVDEIEAWISEKLQTASDESYKDPTNIQSKHQKHQAFEAELHANADRIRGVIDMGNSLIERGACAGSEDAVKARLAALADQWQFLVQKSAEKSQKLKEANKQQNFNTGIKDFDFWLSEVEALLASEDYGKDLASVNNLLKKHQLLEADISAHEDRLKDLNSQADSLMTSSAFDTSQVKDKRDTINGRFQKIKSMAASRRAKLNESHRLHQFFRDMDDEESWIKEKKLLVGSEDYGRDLTGVQNLRKKHKRLEAELAAHEPAIQGVLDTGKKLSDDNTIGKEEIQQRLAQFVEHWKELKQLAAARGQRLEESLEYQQFVANVEEEEAWINEKMTLVASEDYGDTLAAIQGLLKKHEAFETDFTVHKDRVNDVCTNGQDLIKKNNHHEENISSKMKGLNGKVSDLEKAAAQRKAKLDENSAFLQFNWKADVVESWIGEKENSLKTDDYGRDLSSVQTLLTKQETFDAGLQAFQQEGIANITALKDQLLAAKHVQSKAIEARHASLMKRWSQLLANSAARKKKLLEAQSHFRKVEDLFLTFAKKASAFNSWFENAEEDLTDPVRCNSLEEIKALREAHDAFRSSLSSAQADFNQLAELDRQIKSFRVASNPYTWFTMEALEETWRNLQKIIKERELELQKEQRRQEENDKLRQEFAQHANAFHQWIQETRTYLLDGSCMVEESGTLESQLEATKRKHQEIRAMRSQLKKIEDLGAAMEEALILDNKYTEHSTVGLAQQWDQLDQLGMRMQHNLEQQIQARNTTGVTEEALKEFSMMFKHFDKDKSGRLNHQEFKSCLRSLGYDLPMVEEGEPDPEFEAILDTVDPNRDGHVSLQEYMAFMISRETENVKSSEEIESAFRALSSEGKPYVTKEELYQNLTREQADYCVSHMKPYVDGKGRELPTAFDYVEFTRSLFVN

SEQ ID NO: 3 - protein, 2472 aa, Homo sapiens
MDPSGVKVLETAEDIQERRQQVLDRYHRFKELSTLRRQKLEDSYRFQFFQRDAEELEKWIQEKLQIASDENYKDPTNLQGKLQKHQAFEAEVQANSGAIVKLDETGNLMISEGHFASETIRTRLMELHRQWELLLEKMREKGIKLLQAQKLVQYLRECEDVMDWINDKEAIVTSEELGQDLEHVEVLQKKFEEFQTDMAAHEERVNEVNQFAAKLIQEQHPEEELIKTKQDEVNAAWQRLKGLALQRQGKLFGAAEVQRFNRDVDETISWIKEKEQLMASDDFGRDLASVQALLRKHEGLERDLAALEDKVKALCAEADRLQQSHPLSATQIQVKREELITNWEQIRTLAAERHARLNDSYRLQRFLADFRDLTSWVTEMKALINADELASDVAGAEALLDRHQEHKGEIDAHEDSFKSADESGQALLAAGHYASDEVREKLTVLSEERAALLELWELRRQQYEQCMDLQLFYRDTEQVDNWMSKQEAFLLNEDLGDSLDSVEALLKKHEDFEKSLSAQEEKITALDEFATKLIQNNHYAMEDVATRRDALLSRRNALHERAMRRRAQLADSFHLQQFFRDSDELKSWVNEKMKTATDEAYKDPSNLQGKVQKHQAFEAELSANQSRIDALEKAGQKLIDVNHYAKDEVAARMNEVISLWKKLLEATELKGIKLREANQQQQFNRNVEDIELWLYEVEGHLASDDYGKDLTNVQNLQKKHALLEADVAAHQDRIDGITIQARQFQDAGHFDAENIKKKQEALVARYEALKEPMVARKQKLADSLRLQQLFRDVEDEETWIREKEPIAASTNRGKDLIGVQNLLKKHQALQAEIAGHEPRIKAVTQKGNAMVEEGHFAAEDVKAKLHELNQKWEALKAKASQRRQDLEDSLQAQQYFADANEAESWMREKEPIVGSTDYGKDEDSAEALLKKHEALMSDLSAYGSSIQALREQAQSCRQQVAPTDDETGKELVLALYDYQEKSPREVTMKKGDILTLLNSTNKDWWKVEVNDRQGFVPAAYVKKLDPAQSASRENLLEEQGSIALRQEQIDNQTRITKEAGSVSLRMKQVEELYHSLLELGEKRKGMLEKSCKKFMLFREANELQQWINEKEAALTSEEVGADLEQVEVLQKKFDDFQKDLKANESRLKDINKVAEDLESEGLMAEEVQAVQQQEVYGMMPRDETDSKTASPWKSARLMVHTVATFNSIKELNERWRSLQQLAEERSQLLGSAHEVQRFHRDADETKEWIEEKNQALNTDNYGHDLASVQALQRKHEGFERDLAALGDKVNSLGETAERLIQSHPESAEDLQEKCTELNQAWSSLGKRADQRKAKLGDSHDLQRFLSDFRDLMSWINGIRGLVSSDELAKDVTGAEALLERHQEHRTEIDARAGTFQAFEQFGQQLLAHGHYASPEIKQKLDILDQERADLEKAWVQRRMMLDQCLELQLFHRDCEQAENWMAAREAFLNTEDKGDSLDSVEALIKKHEDFDKAINVQEEKIAALQAFADQLIAAGHYAKGDISSRRNEVLDRWRRLKAQMIEKRSKLGESQTLQQFSRDVDEIEAWISEKLQTASDESYKDPTNIQSKHQKHQAFEAELHANADRIRGVIDMGNSLIERGACAGSEDAVKARLAALADQWQFLVQKSAEKSQKLKEANKQQNFNTGIKDFDFWLSEVEALLASEDYGKDLASVNNLLKKHQLLEADISAHEDRLKDLNSQADSLMTSSAFDTSQVKDKRDTINGRFQKIKSMAASRRAKLNESHRLHQFFRDMDDEESWIKEKKLLVGSEDYGRDLTGVQNLRKKHKRLEAELAAHEPAIQGVLDTGKKLSDDNTIGKEEIQQRLAQFVEHWKELKQLAAARGQRLEESLEYQQFVANVEEEEAWINEKMTLVASEDYGDTLAAIQGLLKKHEAFETDFTVHKDRVNDVCTNGQDLIKKNNHHEENISSKMKGLNGKVSDLEKAAAQRKAKLDENSAFLQFNWKADVVESWIGEKENSLKTDDYGRDLSSVQTLLTKQETFDAGLQAFQQEGIANITALKDQLLAAKHVQSKAIEARHASLMKRWSQLLANSAARKKKLLEAQSHFRKVEDLFLTFAKKASAFNSWFENAEEDLTDPVRCNSLEEIKALREAHDAFRSSLSSAQADFNQLAELDRQIKSFRVASNPYTWFTMEALEETWRNLQKIIKERELELQKEQRRQEENDKLRQEFAQHANAFHQWIQETRTYLLDGSCMVEESGTLESQLEATKRKHQEIRAMRSQLKKIEDLGAAMEEALILDNKYTEHSTVGLAQQWDQLDQLGMRMQHNLEQQIQARNTTGVTEEALKEFSMMFKHFDKDKSGRLNHQEFKSCLRSLGYDLPMVEEGEPDPEFEAILDTVDPNRDGHVSLQEYMAFMISRETENVKSSEEIESAFRALSSEGKPYVTKEELYQNLTREQADYCVSHMKPYVDGKGRELPTAFDYVEFTRSLFVN

SEQ ID NO: 4 - protein, 10 aa, Artificial sequence
  Chemically synthesized
ADELASDVAG

SEQ ID NO: 5 - protein, 10 aa, Artificial sequence
  Chemically synthesized
AALTSEEVGA

SEQ ID NO: 6 - protein, 10 aa, Artificial sequence
  Chemically synthesized
QQVAPTDDET

SEQ ID NO: 7 - protein, 10 aa, Artificial sequence
  Chemically synthesized
MMPRDETDSK

SEQ ID NO: 8 - protein, 10 aa, Artificial sequence
  Chemically synthesized
SKTASPWKSA

SEQ ID NO: 9 - protein, 10 aa, Artificial sequence
  Chemically synthesized
YGMMPRDETD

SEQ ID NO: 10 - protein, 10 aa, Artificial sequence
  Chemically synthesized
QEVYGMMPRD

SEQ ID NO: 11 - protein, 10 aa, Artificial sequence
  Chemically synthesized
EVYGMMPRDE

SEQ ID NO: 12 - protein, 10 aa, Artificial sequence
  Chemically synthesized
GMMPRDETDS

SEQ ID NO: 13 - protein, 10 aa, Artificial sequence
  Chemically synthesized
SRETENVKSS

SEQ ID NO: 14 - protein, 10 aa, Artificial sequence
  Chemically synthesized
TEDKGDSLDS

SEQ ID NO: 15 - protein, 10 aa, Artificial sequence
  Chemically synthesized
MVEESGTLES

SEQ ID NO: 16 - protein, 10 aa, Artificial sequence
  Chemically synthesized
RDETDSKTAS

SEQ ID NO: 17 - protein, 10 aa, Artificial sequence
  Chemically synthesized
PRDETDSKTA

SEQ ID NO: 18 - protein, 10 aa, Artificial sequence
  Chemically synthesized
LAEERSQLLG

SEQ ID NO: 19 - protein, 10 aa, Artificial sequence
  Chemically synthesized
NTEDKGDSLD

SEQ ID NO: 20 - protein, 10 aa, Artificial sequence
  Chemically synthesized
VQAVQQQEVY

SEQ ID NO: 21 - protein, 10 aa, Artificial sequence
  Chemically synthesized
SAHEVQRFHR

SEQ ID NO: 22 - protein, 10 aa, Artificial sequence
  Chemically synthesized
AASAGVPYHG

SEQ ID NO: 23 - protein, 10 aa, Artificial sequence
  Chemically synthesized
PVSPVGAEGP

SEQ ID NO: 24 - protein, 10 aa, Artificial sequence
  Chemically synthesized
RSTESAHAAT

SEQ ID NO: 25 - protein, 10 aa, Artificial sequence
  Chemically synthesized
DFDSLEIQGQ

SEQ ID NO: 26 - protein, 10 aa, Artificial sequence
  Chemically synthesized
PDSDWDNDSS

SEQ ID NO: 27 - protein, 10 aa, Artificial sequence
  Chemically synthesized
HLLASADTGR

SEQ ID NO: 28 - protein, 10 aa, Artificial sequence
  Chemically synthesized
TLSRTPEVQS

SEQ ID NO: 29 - protein, 10 aa, Artificial sequence
  Chemically synthesized
HTEMPGTLQA

SEQ ID NO: 30 - protein, 10 aa, Artificial sequence
  Chemically synthesized
QAQLHSDDYG

SEQ ID NO: 31 - protein, 10 aa, Artificial sequence
  Chemically synthesized
QQQEVGMMPR

SEQ ID NO: 32 - protein, 11 aa, Artificial sequence
  Chemically synthesized
QQEVYGMMPRD

SEQ ID NO: 33 - DNA, 7672 bp, Homo sapiens
gaggctcctcggtccttcagcacccctcggcccgacgcacccacgcccctcaccccccgagagccgaaaatggacccaagtggggtcaaagtgctggaaacagcagaggacatccaggagaggcggcagcaggtcctagaccgataccaccgcttcaaggaactctcaacccttaggcgtcagaagctggaagattcctatcgattccagttctttcaaagagatgctgaagagctggagaaatggatacaggaaaaacttcagattgcatctgatgagaattataaagacccaaccaacttgcagggaaagcttcagaagcatcaagcatttgaagctgaagtgcaggccaactcaggagccattgttaagctggatgaaactggaaacctgatgatctcagaagggcattttgcatctgaaaccatacggacccgtttgatggagctgcaccgccagtgggaattacttttggagaagatgcgagaaaaaggaatcaaactgctgcaggcccagaagttggtgcagtacttacgagaatgtgaggacgtgatggactggatcaatgacaaggaagcaattgttacttctgaagagctgggccaggatctggagcatgtagaggttttacagaagaaatttgaagagtttcaaacagatatggctgctcatgaagaaagagttaatgaagtgaaccagtttgctgccaaactcatacaggagcagcaccctgaggaggaactgatcaagactaagcaggatgaagtcaatgcagcctggcagcggctgaagggcctggctctgcagaggcaggggaagctctttggggcagcagaagttcagcgctttaacagggatgtggatgagactatcagttggattaaggaaaaggagcagttaatggcctctgatgattttggccgagacctggcaagtgttcaggctctgcttcggaagcacgagggtctggagagagatcttgctgctctagaagacaaggtcaaagccctgtgtgctgaggctgaccgcctgcaacagtcccaccctctgagtgcaacacagattcaagtgaagcgagaggaactgattacaaactgggagcagatccgcaccttggcggcagagagacatgcacggctcaatgattcatacaggcttcaacgcttccttgctgacttccgtgacctcaccagctgggtgactgagatgaaagccctcatcaatgcagatgagcttgccagtgatgtggctggggctgaagccctgctagatagacaccaagagcacaagggtgaaattgatgcccatgaagacagcttcaaatctgcagatgaatctggacaggcactgcttgctgctggtcactatgcctcagatgaagtgagggagaagctgaccgtcctttccgaggagagagcggcgctgctggagctgtgggagctgcgcaggcagcagtacgagcagtgcatggacctgcagctcttctaccgggacactgagcaggtggacaactggatgagcaagcaggaggcgttcctgttgaatgaagacttgggagattccttggatagtgtggaagcgcttcttaagaagcacgaagactttgagaaatcccttagtgcccaggaggaaaagattacagcattagatgaatttgcaaccaagctaattcagaacaaccactatgcaatggaagatgtggccactcgccgagatgctctgttgagccgccgcaatgcccttcacgagagagccatgcgtcgccgggcccagctagccgattctttccatctgcagcagtttttccgtgattctgatgagctcaagagttgggtcaatgagaagatgaaaactgccacagatgaagcttataaagatccatccaacctacaaggaaaagtacagaagcatcaggcttttgaggctgagctctcagcaaaccagagccgaattgatgccttggagaaagctggccaaaagctgattgatgtcaaccactatgccaaggatgaagtggcagctcgtatgaatgaggtgatcagtttgtggaagaaactgctagaggccactgaactgaaaggaataaagcttcgtgaagccaaccagcaacagcaatttaatcgcaatgttgaggatattgaattgtggctatatgaagtagaaggtcacttggcttcggatgattacggcaaagatcttaccaatgtgcagaacctccagaagaaacatgccctgctagaggcagatgtggctgctcaccaggaccgaattgatggcatcaccattcaggcccgccagttccaagatgctggccattttgatgcagaaaacatcaagaagaaacaggaagccctcgtggctcgctatgaggcactcaaggagcccatggttgcccggaagcagaagctggccgattctctgcggttgcagcagctcttccgggatgttgaggatgaggagacgtggattcgagagaaagagcccattgccgcatctaccaacagaggtaaggatttaattggggtccagaatctgctaaagaaacatcaagccttacaagcagaaattgctggacatgaaccacgcatcaaagcagttacacagaaggggaatgccatggtggaggaaggccattttgctgcagaggatgtgaaggccaagcttcacgagctgaaccaaaagtgggaggcactgaaagccaaagcttcccagcgtcggcaggacctggaggactctctgcaggcccagcagtactttgctgatgctaacgaggctgaatcctggatgcgggagaaggaacccattgtgggcagcactgactatggcaaggacgaagactctgctgaggctctactgaagaaacacgaagctttgatgtcagatctcagtgcctacggcagcagcatccaggctttgcgagaacaagcacagtcctgccggcaacaagtggcccccacggatgatgagactgggaaggagctggtcttggctctctacgactatcaggagaagagtccccgagaggtcaccatgaagaagggagatatccttaccttactcaacagcaccaacaaggattggtggaaagtggaagtgaacgatcgtcagggttttgtgccggctgcgtacgtgaagaaattggaccccgcccagtcagcctcccgggagaatctcctggaggagcaaggcagcatagcactgcggcaggagcagattgacaatcagacacgcataactaaggaggccggcagtgtatctctgcgtatgaagcaggtggaagaactatatcattctctgctggaactgggtgagaagcgtaaaggcatgttggagaagagttgcaagaagtttatgttgttccgtgaagcgaatgaactacagcaatggatcaatgagaaggaagccgctctgacaagtgaggaggtcggagcagacttggagcaggttgaggtgctccagaagaagtttgatgacttccagaaggacctgaaggccaatgagtcacggttgaaggacattaacaaggtagctgaagacctggagtctgaaggtctcatggcagaggaggtgcaggctgtgcaacaacaggaagtgtatggcatgatgcccagggatgaaactgattccaagacagcctccccgtggaagtctgctcgtctgatggttcacaccgtggccacctttaattccatcaaggagctgaatgagcgctggcggtccctacagcagctggccgaggaacggagccagctcttgggcagcgcccatgaagtacagaggttccacagagatgctgatgaaaccaaagaatggattgaagagaagaatcaagctctaaacacagacaattatggacatgatctcgccagtgtccaggccctgcaacgcaagcatgagggcttcgagagggaccttgcggctctcggtgacaaggtaaactcccttggtgaaacagcagagcgcctgatccagtcccatcccgagtcagcagaagacctgcaggaaaagtgcacagagttaaaccaggcctggagcagcctggggaaacgtgcagatcagcgcaaggcaaagttgggtgactcccacgacctgcagcgcttccttagcgatttccgggacctcatgtcttggatcaatggaatacgggggttggtgtcctcagatgagctagccaaggatgtcaccggagctgaggcattgctggagcgacaccaggaacaccggacagaaatcgatgccagggctggcactttccaggcatttgagcagtttggacagcagctgttggctcacggacactatgccagccctgagatcaagcagaaacttgatattcttgaccaggagcgtgcagacctggagaaggcctgggttcagcgcaggatgatgctggatcagtgccttgaactgcagctgttccatcgggactgtgagcaagctgagaactggatggctgcccgggaggccttcttgaataccgaagacaaaggagactcactggacagcgtagaggctctgatcaaaaaacatgaagactttgacaaagcgattaacgtccaggaagagaagattgctgctctgcaggcctttgccgaccagctcatcgctgccggccattatgccaagggagacatttctagccggcgcaatgaggtcttggacaggtggcgacgtctgaaagcccagatgattgagaaaaggtcaaagctaggagaatctcaaaccctccaacagttcagccgggatgtggatgagattgaggcttggatcagtgaaaaattgcaaacagcgagtgatgagtcgtacaaggatcccaccaacatccagctttccaagctgctgagcaagcaccagaagcaccaggcttttgaagcagagctgcatgccaacgctgaccggatccgtggggttatcgacatgggcaactccctcattgaacgtggagcctgtgccggcagtgaggatgctgtcaaggcccgcctggctgccttagctgaccagtggcagttcttggtgcaaaagtcagcggaaaagagccagaaactgaaagaagccaacaagcagcagaacttcaacacagggatcaaggactttgacttctggctgtctgaggtggaggccctgctggcatccgaagattatggcaaagacctggcttctgtgaacaacctgctgaaaaagcatcaactgctggaagcagatatatctgcccatgaggatcgcctgaaggacctgaacagccaggcagacagcctgatgaccagcagtgccttcgacacctcccaagtaaaggacaagagggacaccatcaacgggcgcttccagaagatcaagagcatggcggcctcccggcgagccaagctgaatgaatcccatcgcctgcaccagttcttccgggacatggatgacgaggagtcctggatcaaggagaagaagctgctggtgggctcagaggactacggccgggacctaaccggcgtgcagaacctgaggaagaagcacaagcggctggaagcagaactggctgcgcatgagccggctattcagggtgtcctggacactggcaagaagctgtccgatgacaacaccatcgggaaagaggagatccagcagcggctggcgcagtttgtggagcactggaaagagctgaagcagctggcagctgcccggggtcagcggctggaagagtccttggaatatcagcagtttgtagccaatgtggaagaggaagaagcctggatcaatgagaaaatgaccctggtggccagcgaagattatggcgacactcttgccgccatccagggcttactgaagaaacatgaagcttttgagacagacttcaccgtccacaaggatcgcgtgaatgatgtctgcaccaatggacaagacctcattaagaagaacaatcaccatgaggagaacatctcttcaaagatgaagggcctgaacgggaaagtgtcagacctggagaaagctgcagcccagagaaaggcgaagctggatgagaactcggccttccttcagttcaactggaaggcggacgtggtggagtcctggatcggtgaaaaggagaacagcttgaagacagatgattatggccgagacctgtcttctgtgcagacgctcctcaccaaacaggaaacttttgacgctgggctgcaggccttccagcaggaaggcattgccaacatcactgccctcaaagatcagcttctcgccgccaaacacgttcagtccaaggccatcgaggcccggcacgcctccctcatgaagaggtggagccagcttctggccaactcagccgcccgcaagaagaagcttctggaggctcagagtcacttccgcaaggtggaggacctcttcctgaccttcgccaaaaaggcttctgccttcaacagctggtttgaaaatgcagaggaggacttaacagaccccgtgcgctgcaactccttggaagaaatcaaagctttgcgcgaggcccacgacgccttccgctcctccctcagctctgcccaggctgacttcaaccagctggccgagctggaccgccagatcaagagcttccgcgtagcctccaacccctacacctggtttaccatggaggccctggaggagacctggaggaacctacagaaaatcatcaaggagagggagctggagctgcagaaggaacagcggcggcaggaggagaacgacaagctgcgccaggagtttgcccagcacgccaacgccttccaccagtggatccaagagaccaggacatacctcctcgatgggtcctgtatggtggaagagtcggggaccctcgaatcccagcttgaagctaccaaacgcaagcaccaggaaatccgagccatgagaagtcagctcaaaaagatcgaggacctgggggccgccatggaggaggccctcatcctggacaacaagtacacggagcacagcaccgtgggcctcgcccagcagtgggaccagctggaccagctgggcatgcgcatgcagcacaacctggagcagcagatccaggccaggaacacaacaggtgtgactgaggaggccctcaaagaattcagcatgatgtttaaacactttgacaaggacaagtctggcaggctgaaccatcaggagttcaaatcttgcctgcgctccctgggctatgacctgcccatggtggaggaaggggaacctgaccctgagttcgaggcaatcctggacacggtggatccgaacagagatggccatgtctccttgcaagaatacatggctttcatgatcagccgcgaaactgagaacgtcaagtccagcgaggagattgagagcgccttccgggccctcagctcagagggaaagccttacgtgaccaaggaggagctctaccagaacctgacccgggaacaagccgactactgcgtctcccacatgaagccctacgtggacggcaagggccgcgagctccccaccgcgttcgactacgtggagttcacccgctcgcttttcgtgaactgagccactccctgggtcacccacccctcgctgcttgccctgcgtcgccttgctgcatgtccgctcctctgtgtgctctcactttccactgtaaccttaagcctgcttagcttggaataagacttaggagaaaatggtgcttcactaacccgcttccggtccagtcacaatc